SEQUENCE LISTING (38 sequences)

SEQ ID NO: 1 - DNA, 1659 bp, synthetic construct
  source 1..1659: mol_type = other DNA
  misc_feature 1..1659: note = Human p16 promoter
gcgggccacatttccaggttcccggagctgggcctagtcccgaatcctctggcacacacccacccactcaggccgcgggtccagcccgcgaggtttaggacggatccaggcagaccgcaggctccgggtcggggcaccgggtcagcgcgccggcctgaaggcggcgtcctgggctcgacttcccgcgcgcggagagccggcgagcccgcgtccgagttcctggacgagagccgagcctcgcttagaccgcgctcaggacccggctcctccgcattctccggctgcccctgtgtcctcgactcacccctcctttctgccgctccttcctttccttgccctgcttttactgttcccaaacaggaccgcttttcctgtctcccagctggaaaggaggaagggagagagtccagaaaggatcggtgatgtggaagaaaaggggaggaggggacatggagggggagaccggagagagaacgtacgccgaggagtcaggcggcgggatcaaggggagtcggggtgtctgggcgcggggcagagcgtggaggcggcagcggccaacggtcgccaagacaaccattctacgcgaggacgcggcgacaggaggggagcggccagcaggggaggggagcgcgggggaagaggaaagaggaagaagcgctcagatgctccgcggctgtcgtgaaggttaaaaccgaaaataaaaatgggctagacacaaaggactcggtgcttgtcccagccaggcgccctcggcgacgcgggcagctgggaggggaatgggcgcccggacccagctgggacccccgggtgcgactccacctacctagtccggcgccaggccgggtcgacagctccggcagcgccagcgccgcgccgtgtccagatgtcgcgtcagaggcgtgcagcggtttagtttaatttcgcttgttttccaaatctagaagaggagcggagcggcttttagttcaaaactgacattcagcctcctgattggcggatagagcaatgagatgacctcgctttcctttcttcctttttcatttttaaataatctagtttgaagaatggaagactttcgacgaggggagccaggaataaaataaggggaataggggagcggggacgcgagcagcaccagaatccgcgggagcgcggctgttcctggtagggccgtgtcaggtgacggatgtagctagggggcgagctgcctggagttgcgttccaggcgtccggcccctgggccgtcaccgcggggcgcccgcgctgagggtgggaagatggtggtgggggtgggggcgcacacagggcgggaaagtggcggtaggcgggagggagaggaacgcgggccctgagccgcccgcgcgcgcgcctccctacgggcgcctccggcagcccttcccgcgtgcgcagggctcagagccgttccgagatcttggaggtccgggtgggagtgggggtggggtgggggtgggggtgaaggtggggggcgggcgcgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtg

SEQ ID NO: 2 - DNA, 900 bp, synthetic construct
  source 1..900: mol_type = other DNA
  misc_feature 1..900: note = Mouse p16 promoter
ttttcctgtttcgcgggggccggggtggaggcggagaaggaggcgaggaggcagcggagaagggggcagccgggaggggggaggggaccggagaaggaggcgacgcggggcggggaaagaggaagagccggggatgctcgcgcttaaaaccgaaaataaacaacgtcctcgggctgcggcctcgtgtctgcccgaccgcgcgggggcctggccgggacacacttaccggtccgccgcggcgtgcaggccctggtcccgggtacacgcagcggctcttctctgcgcgctgttcccaaccggaggagcgcagcgtaccgctaagggttcaaaacgcccgcgcgcgtcctgattggcgggccggcccatggctggcctcgccgatcttcctattttcttctttttatagatggactcggagcaagggaaacctcgccgaggggcgtcaggcatagacgaaagtttcgggacccgaaagttaaccggagcgaaagccgatcggcgcaggagacgttgccggccggctgcgaaagggcgcagctactgctagggggcgggcggtgcgcacgcgtcccgcggcgctggctgtcaccgcgatgggtggcgagcgaagcgagcgggatccggagcgtgccctgcgcgggaggcagcgggaccccggatgcggcagggcccgcgcgcgcctccccctgggcgcctctgggaagctttcccgcgcgactggggaccgcgcgccttgggactcgggggcgcgcctgagggcggagatgggcgtggagcaaagatgggccgggggcggcgcgtgggtctcgaggtgcctcaacgccgaaggggctgggggcggcgcttctcacctcgcttgtcacagtgaggccgccgctgagggagtacagcagcgggagc

SEQ ID NO: 3 - DNA, 1080 bp, synthetic construct
  source 1..1080: mol_type = other DNA
  misc_feature 1..1080: note = Human OCT4 / POU5F1
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaac

SEQ ID NO: 4 - DNA, 951 bp, synthetic construct
  source 1..951: mol_type = other DNA
  misc_feature 1..951: note = Human SOX2
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatg

SEQ ID NO: 5 - DNA, 1413 bp, synthetic construct
  source 1..1413: mol_type = other DNA
  misc_feature 1..1413: note = Human KLF4
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 6 - protein, 5 aa, synthetic construct
  REGION 1..5: note = RXK(R)R
RXKRR

SEQ ID NO: 7 - DNA, 951 bp, synthetic construct
  source 1..951: mol_type = other DNA
  misc_feature 1..951: note = dnNFKBIA (h-IKB alpha sup repressor)
atgttccaggcggccgagcgtccccaggagtgggccatggagggcccccgcgacgggctgaagaaggagcggctactggacgaccgccacgacgccggcctggacgccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgtta

SEQ ID NO: 8 - DNA, 568 bp, synthetic construct
  source 1..568: mol_type = other DNA
  misc_feature 1..568: note = IRES
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacga

SEQ ID NO: 9 - DNA, 6651 bp, synthetic construct
  source 1..6651: mol_type = other DNA
  misc_feature 1..6651: note = AAV CAG-CBRLuc-GFP
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatggtaaagcgtgagaaaaatgtcatctatggccctgagcctctccatcctttggaggatttgactgccggcgaaatgctgtttcgtgctctccgcaagcactctcatttgcctcaagccttggtcgatgtggtcggcgatgaatctttgagctacaaggagttttttgaggcaaccgtcttgctggctcagtccctccacaattgtggctacaagatgaacgacgtcgttagtatctgtgctgaaaacaatacccgtttcttcattccagtcatcgccgcatggtatatcggtatgatcgtggctccagtcaacgagagctacattcccgacgaactgtgtaaagtcatgggtatctctaagccacagattgtcttcaccactaagaatattctgaacaaagtcctggaagtccaaagccgcaccaactttattaagcgtatcatcatcttggacactgtggagaatattcacggttgcgaatctttgcctaatttcatctctcgctattcagacggcaacatcgcaaactttaaaccactccacttcgaccctgtggaacaagttgcagccattctgtgtagcagcggtactactggactcccaaagggagtcatgcagacccatcaaaacatttgcgtgcgtctgatccatgctctcgatccacgctacggcactcagctgattcctggtgtcaccgtcttggtctacttgcctttcttccatgctttcggctttcatattactttgggttactttatggtcggtctccgcgtgattatgttccgccgttttgatcaggaggctttcttgaaagccatccaagattatgaagtccgcagtgtcatcaacgtgcctagcgtgatcctgtttttgtctaagagcccactcgtggacaagtacgacttgtcttcactgcgtgaattgtgttgcggtgccgctccactggctaaggaggtcgctgaagtggccgccaaacgcttgaatcttccagggattcgttgtggcttcggcctcaccgaatctaccagtgcgattatccagactctcggggatgagtttaagagcggctctttgggccgtgtcactccactcatggctgctaagatcgctgatcgcgaaactggtaaggctttgggcccgaaccaagtgggcgagctgtgtatcaaaggccctatggtgagcaagggttatgtcaataacgttgaagctaccaaggaggccatcgacgacgacggctggttgcattctggtgattttggatattacgacgaagatgagcatttttacgtcgtggatcgttacaaggagctgatcaaatacaagggtagccaggttgctccagctgagttggaggagattctgttgaaaaatccatgcattcgcgatgtcgctgtggtcggcattcctgatctggaggccggcgaactgccttctgctttcgttgtcaagcagcctggtacagaaattaccgccaaagaagtgtatgattacctggctgaacgtgtgagccatactaagtacttgcgtggcggcgtgcgttttgttgactccatccctcgtaacgtaacaggcaaaattacccgcaaggagctgttgaaacaattgttggtgaaggccggcggtggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgagtgaggccgccgctgagggagtacagcagcgggagcgccacc

SEQ ID NO: 10 - DNA, 8483 bp, synthetic construct
  source 1..8483: mol_type = other DNA
  misc_feature 1..8483: note = AAV H-p16 OSK shortWPRE sv40 polya
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagcgggccacatttccaggttcccggagctgggcctagtcccgaatcctctggcacacacccacccactcaggccgcgggtccagcccgcgaggtttaggacggatccaggcagaccgcaggctccgggtcggggcaccgggtcagcgcgccggcctgaaggcggcgtcctgggctcgacttcccgcgcgcggagagccggcgagcccgcgtccgagttcctggacgagagccgagcctcgcttagaccgcgctcaggacccggctcctccgcattctccggctgcccctgtgtcctcgactcacccctcctttctgccgctccttcctttccttgccctgcttttactgttcccaaacaggaccgcttttcctgtctcccagctggaaaggaggaagggagagagtccagaaaggatcggtgatgtggaagaaaaggggaggaggggacatggagggggagaccggagagagaacgtacgccgaggagtcaggcggcgggatcaaggggagtcggggtgtctgggcgcggggcagagcgtggaggcggcagcggccaacggtcgccaagacaaccattctacgcgaggacgcggcgacaggaggggagcggccagcaggggaggggagcgcgggggaagaggaaagaggaagaagcgctcagatgctccgcggctgtcgtgaaggttaaaaccgaaaataaaaatgggctagacacaaaggactcggtgcttgtcccagccaggcgccctcggcgacgcgggcagctgggaggggaatgggcgcccggacccagctgggacccccgggtgcgactccacctacctagtccggcgccaggccgggtcgacagctccggcagcgccagcgccgcgccgtgtccagatgtcgcgtcagaggcgtgcagcggtttagtttaatttcgcttgttttccaaatctagaagaggagcggagcggcttttagttcaaaactgacattcagcctcctgattggcggatagagcaatgagatgacctcgctttcctttcttcctttttcatttttaaataatctagtttgaagaatggaagactttcgacgaggggagccaggaataaaataaggggaataggggagcggggacgcgagcagcaccagaatccgcgggagcgcggctgttcctggtagggccgtgtcaggtgacggatgtagctagggggcgagctgcctggagttgcgttccaggcgtccggcccctgggccgtcaccgcggggcgcccgcgctgagggtgggaagatggtggtgggggtgggggcgcacacagggcgggaaagtggcggtaggcgggagggagaggaacgcgggccctgagccgcccgcgcgcgcgcctccctacgggcgcctccggcagcccttcccgcgtgcgcagggctcagagccgttccgagatcttggaggtccgggtgggagtgggggtggggtgggggtgggggtgaaggtggggggcgggcgcgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtgatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacggatccggagccacgaacttctctctgttaaagcaagcaggagatgttgaagaaaaccccgggcctatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggatccggagccacgaacttctctctgttaaagcaagcaggagatgttgaagaaaaccccgggcctatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 11 - DNA, 6641 bp, synthetic construct
  source 1..6641: mol_type = other DNA
  misc_feature 1..6641: note = AAV H-p16 IKKB sup repressor-GFP shortWPRE sv40 polya
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagcgggccacatttccaggttcccggagctgggcctagtcccgaatcctctggcacacacccacccactcaggccgcgggtccagcccgcgaggtttaggacggatccaggcagaccgcaggctccgggtcggggcaccgggtcagcgcgccggcctgaaggcggcgtcctgggctcgacttcccgcgcgcggagagccggcgagcccgcgtccgagttcctggacgagagccgagcctcgcttagaccgcgctcaggacccggctcctccgcattctccggctgcccctgtgtcctcgactcacccctcctttctgccgctccttcctttccttgccctgcttttactgttcccaaacaggaccgcttttcctgtctcccagctggaaaggaggaagggagagagtccagaaaggatcggtgatgtggaagaaaaggggaggaggggacatggagggggagaccggagagagaacgtacgccgaggagtcaggcggcgggatcaaggggagtcggggtgtctgggcgcggggcagagcgtggaggcggcagcggccaacggtcgccaagacaaccattctacgcgaggacgcggcgacaggaggggagcggccagcaggggaggggagcgcgggggaagaggaaagaggaagaagcgctcagatgctccgcggctgtcgtgaaggttaaaaccgaaaataaaaatgggctagacacaaaggactcggtgcttgtcccagccaggcgccctcggcgacgcgggcagctgggaggggaatgggcgcccggacccagctgggacccccgggtgcgactccacctacctagtccggcgccaggccgggtcgacagctccggcagcgccagcgccgcgccgtgtccagatgtcgcgtcagaggcgtgcagcggtttagtttaatttcgcttgttttccaaatctagaagaggagcggagcggcttttagttcaaaactgacattcagcctcctgattggcggatagagcaatgagatgacctcgctttcctttcttcctttttcatttttaaataatctagtttgaagaatggaagactttcgacgaggggagccaggaataaaataaggggaataggggagcggggacgcgagcagcaccagaatccgcgggagcgcggctgttcctggtagggccgtgtcaggtgacggatgtagctagggggcgagctgcctggagttgcgttccaggcgtccggcccctgggccgtcaccgcggggcgcccgcgctgagggtgggaagatggtggtgggggtgggggcgcacacagggcgggaaagtggcggtaggcgggagggagaggaacgcgggccctgagccgcccgcgcgcgcgcctccctacgggcgcctccggcagcccttcccgcgtgcgcagggctcagagccgttccgagatcttggaggtccgggtgggagtgggggtggggtgggggtgggggtgaaggtggggggcgggcgcgctcagggaaggcgggtgcgcgcctgcggggcggagatgggcagggggcggtgcgtgggtcccagtctgcagttaagggggcaggagtggcgctgctcacctctggtgccaaagggcggcgcagcggctgccgagctcggccctggaggcggcgagaacatggtgatgttccaggcggccgagcgtccccaggagtgggccatggagggcccccgcgacgggctgaagaaggagcggctactggacgaccgccacgacgccggcctggacgccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttaggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 12 - DNA, 5888 bp, synthetic construct
  source 1..5888: mol_type = other DNA
  misc_feature 1..5888: note = AAV mp16 h-IKB alpha super repressor-GFP
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagattttcctgtttcgcgggggccggggtggaggcggagaaggaggcgaggaggcagcggagaagggggcagccgggaggggggaggggaccggagaaggaggcgacgcggggcggggaaagaggaagagccggggatgctcgcgcttaaaaccgaaaataaacaacgtcctcgggctgcggcctcgtgtctgcccgaccgcgcgggggcctggccgggacacacttaccggtccgccgcggcgtgcaggccctggtcccgggtacacgcagcggctcttctctgcgcgctgttcccaaccggaggagcgcagcgtaccgctaagggttcaaaacgcccgcgcgcgtcctgattggcgggccggcccatggctggcctcgccgatcttcctattttcttctttttatagatggactcggagcaagggaaacctcgccgaggggcgtcaggcatagacgaaagtttcgggacccgaaagttaaccggagcgaaagccgatcggcgcaggagacgttgccggccggctgcgaaagggcgcagctactgctagggggcgggcggtgcgcacgcgtcccgcggcgctggctgtcaccgcgatgggtggcgagcgaagcgagcgggatccggagcgtgccctgcgcgggaggcagcgggaccccggatgcggcagggcccgcgcgcgcctccccctgggcgcctctgggaagctttcccgcgcgactggggaccgcgcgccttgggactcgggggcgcgcctgagggcggagatgggcgtggagcaaagatgggccgggggcggcgcgtgggtctcgaggtgcctcaacgccgaaggggctgggggcggcgcttctcacctcgcttgtcacagtgaggccgccgctgagggagtacagcagcgggagcgccaccatgttccaggcggccgagcgtccccaggagtgggccatggagggcccccgcgacgggctgaagaaggagcggctactggacgaccgccacgacgccggcctggacgccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttaggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 13 - DNA, 7772 bp, synthetic construct
  source 1..7772: mol_type = other DNA
  misc_feature 1..7772: note = AAV mp16-OSK
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagattttcctgtttcgcgggggccggggtggaggcggagaaggaggcgaggaggcagcggagaagggggcagccgggaggggggaggggaccggagaaggaggcgacgcggggcggggaaagaggaagagccggggatgctcgcgcttaaaaccgaaaataaacaacgtcctcgggctgcggcctcgtgtctgcccgaccgcgcgggggcctggccgggacacacttaccggtccgccgcggcgtgcaggccctggtcccgggtacacgcagcggctcttctctgcgcgctgttcccaaccggaggagcgcagcgtaccgctaagggttcaaaacgcccgcgcgcgtcctgattggcgggccggcccatggctggcctcgccgatcttcctattttcttctttttatagatggactcggagcaagggaaacctcgccgaggggcgtcaggcatagacgaaagtttcgggacccgaaagttaaccggagcgaaagccgatcggcgcaggagacgttgccggccggctgcgaaagggcgcagctactgctagggggcgggcggtgcgcacgcgtcccgcggcgctggctgtcaccgcgatgggtggcgagcgaagcgagcgggatccggagcgtgccctgcgcgggaggcagcgggaccccggatgcggcagggcccgcgcgcgcctccccctgggcgcctctgggaagctttcccgcgcgactggggaccgcgcgccttgggactcgggggcgcgcctgagggcggagatgggcgtggagcaaagatgggccgggggcggcgcgtgggtctcgaggtgcctcaacgccgaaggggctgggggcggcgcttctcacctcgcttgtcacagtgaggccgccgctgagggagtacagcagcgggagcgccaccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacgctagcggcagcggcgccacgaacttctctctgttaaagcaagcaggagatgttgaagaaaaccccgggcctgcatgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatggcatgcggctccggcgagggcaggggaagtcttctaacatgcggggacgtggaggaaaatcccggcccactcgagatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcacttttaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 14 - DNA, 6610 bp, synthetic construct
  source 1..6610: mol_type = other DNA
  misc_feature 1..6610: note = AAV mp16 CBRLuc-GFP
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagattttcctgtttcgcgggggccggggtggaggcggagaaggaggcgaggaggcagcggagaagggggcagccgggaggggggaggggaccggagaaggaggcgacgcggggcggggaaagaggaagagccggggatgctcgcgcttaaaaccgaaaataaacaacgtcctcgggctgcggcctcgtgtctgcccgaccgcgcgggggcctggccgggacacacttaccggtccgccgcggcgtgcaggccctggtcccgggtacacgcagcggctcttctctgcgcgctgttcccaaccggaggagcgcagcgtaccgctaagggttcaaaacgcccgcgcgcgtcctgattggcgggccggcccatggctggcctcgccgatcttcctattttcttctttttatagatggactcggagcaagggaaacctcgccgaggggcgtcaggcatagacgaaagtttcgggacccgaaagttaaccggagcgaaagccgatcggcgcaggagacgttgccggccggctgcgaaagggcgcagctactgctagggggcgggcggtgcgcacgcgtcccgcggcgctggctgtcaccgcgatgggtggcgagcgaagcgagcgggatccggagcgtgccctgcgcgggaggcagcgggaccccggatgcggcagggcccgcgcgcgcctccccctgggcgcctctgggaagctttcccgcgcgactggggaccgcgcgccttgggactcgggggcgcgcctgagggcggagatgggcgtggagcaaagatgggccgggggcggcgcgtgggtctcgaggtgcctcaacgccgaaggggctgggggcggcgcttctcacctcgcttgtcacagtgaggccgccgctgagggagtacagcagcgggagcgccaccatggatggtaaagcgtgagaaaaatgtcatctatggccctgagcctctccatcctttggaggatttgactgccggcgaaatgctgtttcgtgctctccgcaagcactctcatttgcctcaagccttggtcgatgtggtcggcgatgaatctttgagctacaaggagttttttgaggcaaccgtcttgctggctcagtccctccacaattgtggctacaagatgaacgacgtcgttagtatctgtgctgaaaacaatacccgtttcttcattccagtcatcgccgcatggtatatcggtatgatcgtggctccagtcaacgagagctacattcccgacgaactgtgtaaagtcatgggtatctctaagccacagattgtcttcaccactaagaatattctgaacaaagtcctggaagtccaaagccgcaccaactttattaagcgtatcatcatcttggacactgtggagaatattcacggttgcgaatctttgcctaatttcatctctcgctattcagacggcaacatcgcaaactttaaaccactccacttcgaccctgtggaacaagttgcagccattctgtgtagcagcggtactactggactcccaaagggagtcatgcagacccatcaaaacatttgcgtgcgtctgatccatgctctcgatccacgctacggcactcagctgattcctggtgtcaccgtcttggtctacttgcctttcttccatgctttcggctttcatattactttgggttactttatggtcggtctccgcgtgattatgttccgccgttttgatcaggaggctttcttgaaagccatccaagattatgaagtccgcagtgtcatcaacgtgcctagcgtgatcctgtttttgtctaagagcccactcgtggacaagtacgacttgtcttcactgcgtgaattgtgttgcggtgccgctccactggctaaggaggtcgctgaagtggccgccaaacgcttgaatcttccagggattcgttgtggcttcggcctcaccgaatctaccagtgcgattatccagactctcggggatgagtttaagagcggctctttgggccgtgtcactccactcatggctgctaagatcgctgatcgcgaaactggtaaggctttgggcccgaaccaagtgggcgagctgtgtatcaaaggccctatggtgagcaagggttatgtcaataacgttgaagctaccaaggaggccatcgacgacgacggctggttgcattctggtgattttggatattacgacgaagatgagcatttttacgtcgtggatcgttacaaggagctgatcaaatacaagggtagccaggttgctccagctgagttggaggagattctgttgaaaaatccatgcattcgcgatgtcgctgtggtcggcattcctgatctggaggccggcgaactgccttctgctttcgttgtcaagcagcctggtacagaaattaccgccaaagaagtgtatgattacctggctgaacgtgtgagccatactaagtacttgcgtggcggcgtgcgttttgttgactccatccctcgtaacgtaacaggcaaaattacccgcaaggagctgttgaaacaattgttggtgaaggccggcggtggatccggagagggcagaggaagtcttctaacatgcggtgacgtggaggagaatcccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagtttcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgagtgaggccgccgctgagggagtacagcagcgggagcgccacc

SEQ ID NO: 15 - DNA, 21 bp, synthetic construct
  source 1..21: mol_type = other DNA
  misc_feature 1..21: note = ITR-F
ggaacccctagtgatggagtt

SEQ ID NO: 16 - DNA, 16 bp, synthetic construct
  source 1..16: mol_type = other DNA
  misc_feature 1..16: note = ITR-R
cggcctcagtgagcga

SEQ ID NO: 17 - DNA, 18 bp, synthetic construct
  source 1..18: mol_type = other DNA
  misc_feature 1..18: note = Cdkn2a FP
gtgtgcatgacgtgcggg

SEQ ID NO: 18 - DNA, 23 bp, synthetic construct
  source 1..23: mol_type = other DNA
  misc_feature 1..23: note = Cdkn2a RP
gttgcccatcatcatcacctgaa

SEQ ID NO: 19 - DNA, 20 bp, synthetic construct
  source 1..20: mol_type = other DNA
  misc_feature 1..20: note = Ctcf FP
gcctgcagccactgcgacaa

SEQ ID NO: 20 - DNA, 21 bp, synthetic construct
  source 1..21: mol_type = other DNA
  misc_feature 1..21: note = Ctcf RP
ccattgtgttccggcgggtga

SEQ ID NO: 21 - DNA, 20 bp, synthetic construct
  source 1..20: mol_type = other DNA
  misc_feature 1..20: note = Il1a FP
aggagaagaccagcccgtgt

SEQ ID NO: 22 - DNA, 20 bp, synthetic construct
  source 1..20: mol_type = other DNA
  misc_feature 1..20: note = Il1a RP
tgccaggtgcacccgacttt

SEQ ID NO: 23 - DNA, 20 bp, synthetic construct
  source 1..20: mol_type = other DNA
  misc_feature 1..20: note = Il1b FP
aaagacggcacacccaccct

SEQ ID NO: 24 - DNA, 22 bp, synthetic construct
  source 1..22: mol_type = other DNA
  misc_feature 1..22: note = Il1b RP
tgtgctctgcttgtgaggtgct

SEQ ID NO: 25 - DNA, 20 bp, synthetic construct
  source 1..20: mol_type = other DNA
  misc_feature 1..20: note = Oct4 FP
cagcagatcactcacatcgc

SEQ ID NO: 26 - DNA, 20 bp, synthetic construct
  source 1..20: mol_type = other DNA
  misc_feature 1..20: note = Oct4 RP
cgccggttacagaaccatac

SEQ ID NO: 27 - DNA, 20 bp, synthetic construct
  source 1..20: mol_type = other DNA
  misc_feature 1..20: note = Sox2 FP
cctgggcgcggagtggaaac

SEQ ID NO: 28 - DNA, 20 bp, synthetic construct
  source 1..20: mol_type = other DNA
  misc_feature 1..20: note = Sox2 RP
ccgccgcggccggtatttat

SEQ ID NO: 29 - DNA, 23 bp, synthetic construct
  source 1..23: mol_type = other DNA
  misc_feature 1..23: note = CTCF FP
gggcttgagagctgggttctatt

SEQ ID NO: 30 - DNA, 23 bp, synthetic construct
  source 1..23: mol_type = other DNA
  misc_feature 1..23: note = CTCFRP
cttcgactgcatcaccttccatt

SEQ ID NO: 31 - DNA, 25 bp, synthetic construct
  source 1..25: mol_type = other DNA
  misc_feature 1..25: note = TBP FP
gtgaacatcatggatcagaacaaca

SEQ ID NO: 32 - DNA, 23 bp, synthetic construct
  source 1..23: mol_type = other DNA
  misc_feature 1..23: note = TBP RP
aagatagggattccgggagtcat

SEQ ID NO: 33 - DNA, 22 bp, synthetic construct
  source 1..22: mol_type = other DNA
  misc_feature 1..22: note = CDKN2A FP
acattcatgtgggcatttcttg

SEQ ID NO: 34 - DNA, 23 bp, synthetic construct
  source 1..23: mol_type = other DNA
  misc_feature 1..23: note = CDKN2A RP
aatgcttgtcatgaagtcgacag

SEQ ID NO: 35 - DNA, 23 bp, synthetic construct
  source 1..23: mol_type = other DNA
  misc_feature 1..23: note = IL1A FP
ccagtgctgctgaaggagatgcc

SEQ ID NO: 36 - DNA, 20 bp, synthetic construct
  source 1..20: mol_type = other DNA
  misc_feature 1..20: note = IL1A RP
ccctgccaagcacacccagt

SEQ ID NO: 37 - DNA, 22 bp, synthetic construct
  source 1..22: mol_type = other DNA
  misc_feature 1..22: note = IL1B FP
gctctccacctccagggacagg

SEQ ID NO: 38 - DNA, 20 bp, synthetic construct
  source 1..20: mol_type = other DNA
  misc_feature 1..20: note = 1L1B RP
tgaggcccaaggccacaggt

(19) United States
(12) Patent Application Publication
(10) Pub. No.: US 2026/0337241 A1
(43) Pub. Date: Oct. 29, 2026

(54) EXPRESSION OF REGENERATION FACTORS IN AGED/SENESCENT CELLS

(71) Applicant: Altos Labs, Inc., Redwood City, CA (US)

(72) Inventors: Sanjeeb Kumar SAHU, San Diego, CA (US); Pradeep Reddy DUBBAKA VENU, San Diego, CA (US); Juan Carlos IZPISUA BELMONTE, La Jolla, CA (US)

(73) Assignee: Altos Labs, Inc., Redwood City, CA (US)

(21) Appl. No.: 19/115,879

(22) PCT Filed: Sep. 28, 2023
(86) PCT No.: PCT/US2023/075374

§ 371 Date: Mar. 27, 2025

Related U.S. Application Data
(60) Provisional application No. 63/377,509, filed on Sep. 28, 2022.
(60) Provisional application No. 63/493,913, filed on Apr. 3, 2023.

(51) Int. Cl. C07K 14/47; A61K 38/17; A61K 48/00; C12N 15/86

(52) U.S. Cl. CPC C07K 14/4703; A61K 38/1709; A61K 48/005; C07K 14/4702; C12N 15/86; C12N 2750/14143; C12N 2750/14151; C12N 2830/48; C12N 2840/203

(57) ABSTRACT

Provided are materials and methods for rejuvenating senescent cells. Senescent cells are transduced with a viral vector to express Oct4, Sox2, and Klf4 proteins for partial cell reprogramming and a dominant negative NFκBIA protein to suppress a senescence-associated secretory phenotype. The materials and methods provided can be used to treat progeria syndrome, signs and symptoms of premature aging or natural aging or to rejuvenate tissue in subjects experiencing premature aging or having an age-related disease.

### CROSS-REFERENCE TO RELATED APPLICATIONS

This application claims the benefit of U.S. Provisional Application No. 63/377,509, filed Sep. 28, 2022, and U.S. Provisional Application No. 63/493,913, filed Apr. 3, 2023, which are incorporated by reference herein in their entirety.

### REFERENCE TO SEQUENCE LISTING SUBMITTED ELECTRONICALLY

The content of the electronically submitted sequence listing in ASCII text file (Name 4967_020PC02_SequenceListing_ST26.xml; Size: 98,303 bytes; and Date of Creation: Aug. 31, 2023) filed with the application is incorporated herein by reference in its entirety.

### BACKGROUND

Aging is a complex process associated with genomic and epigenomic alterations. Mouse genetic studies have shown that disruption of genes involved in genome maintenance can lead to a shortened lifespan, and genetic or pharmacological intervention of, e.g., the insulin signaling pathway extends longevity in multiple mouse models (Folgueras, A. R., Freitas-Rodriguez, S., Velasco, G. & Lopez-Otin, C. Mouse Models to Disentangle the Hallmarks of Human Aging. Circ Res 123, 905-924, 2018; Grunewald, M. et al. Counteracting age-related VEGF signaling insufficiency promotes healthy aging and extends life span. Science 373, 2021). Other methods, including caloric restriction and heterochronic parabiosis have shown the potential to improve health-span in mice and monkeys (Harrison, D. E. et al. Rapamycin fed late in life extends lifespan in genetically heterogeneous mice. Nature 460, 392-395, 2009; Colman, R. J. et al. Caloric restriction delays disease onset and mortality in rhesus monkeys. Science 325, 201-204, 2009).

Besides a permanent growth arrest phenotype of senescent cells, chronic DNA damage signaling can drive the expression and secretion of several cytokines, chemokines, growth factors, and proteases; collectively, these features are termed senescence-associated secretory phenotype (SASP) that have deleterious effects on the tissue microenvironment (Coppe, J. P., Desprez, P. Y., Krtolica, A. & Campisi, J. The senescence-associated secretory phenotype: the dark side of tumor suppression. Annu Rev Pathol 5, 99-118, 2010; Kang, C. et al. The DNA damage response induces inflammation and senescence by inhibiting autophagy of GATA4. Science 349, 2015; Gorgoulis, V. et al. Cellular Senescence: Defining a Path Forward. Cell 179, 813-827, 2019; Herranz, N. & Gil, J. Mechanisms and functions of cellular senescence. J Clin Invest 128, 1238-1246, 2018). SASP can also lead to deterioration of the functional competence of stem cells, thus compromising their role in tissue renewal (Lopez-Otin, C., Blasco, M. A., Partridge, L., Serrano, M. & Kroemer, G. The hallmarks of aging. Cell 153, 1194-1217, 2013).

Major limitations to developing clinical applications of treatments aimed at rejuvenation are the lack of understanding what the appropriate cell types or cell states are to be targeted. Further, it is unknown whether young cells can be expanded at the expense of old ones to yield functional rejuvenation phenotypes at the organ and organism levels without simply depleting organs and organisms of senescent cells thereby jeopardizing their structural integrities.

### BRIEF SUMMARY

Provided are polynucleotides comprising a senescent-cell specific promoter operably linked to a nucleic acid sequence encoding an Oct4 protein, and/or a nucleic acid sequence encoding a Sox2 protein, and/or a nucleic acid sequence encoding a Klf4 protein.

In some aspects, the senescent-cell specific promoter is a CDKN2A (p16) promoter.

In some aspects, the polynucleotides comprise at least one p16 promoter operably linked to a nucleic acid sequence encoding an Oct4 protein, and/or a nucleic acid sequence encoding a Sox2 protein, and/or a nucleic acid sequence encoding a Klf4 protein.

In some aspects, the p16 promoter is operably linked to a nucleic acid sequence encoding an Oct4 protein, a Sox2 protein, and a Klf4 protein.

In some aspects, the p16 promoter is a human or murine p16 promoter.

In some aspects, the human p16 promoter has a nucleotide sequence at least 85%, 86%, 87%, 88%, 89%, 90%, 91%, 92%, 93%, 94%, 95%, 96%, 97%, 98%, 99%, or 100% identical to SEQ ID NO: 1.

In some aspects, the murine p16 promoter has a nucleotide sequence at least 85%, 86%, 87%, 88%, 89%, 90%, 91%, 92%, 93%, 94%, 95%, 96%, 97%, 98%, 99%, or 100% identical to SEQ ID NO: 2.

In some aspects, the polynucleotide encodes a human Oct4 protein, and/or a human Sox2 protein, and/or a human Klf4 protein.

In some aspects, the nucleic acid sequence encoding Oct4 comprises SEQ ID NO: 3, the nucleic acid sequence encoding Sox2 comprises SEQ ID NO: 4, and the nucleic acid encoding Klf4 comprises SEQ ID NO: 5.

In some aspects, the polynucleotide further comprises at least one Woodchuck Hepatitis Virus Posttranscriptional Regulatory Element (WPRE) sequence and at least one polyadenylation signal sequence.

In some aspects, the at least one polyadenylation signal sequence is an SV40 polyadenylation signal sequence, a human growth hormone polyadenylation signal sequence, or a bovine growth hormone polyadenylation signal sequence.

In some aspects, the polynucleotide further comprises at least one internal ribosome entry site (IRES).

In some aspects, the polynucleotide further comprises at least one proteolytic cleavage site.

In some aspects, the at least one proteolytic cleavage site is a self-processing cleavage site or a furin cleavage site.

In some aspects, the self-processing cleavage site is a P2A, E2A, F2A, or T2A peptide.

In some aspects, the furin cleavage site comprises the consensus sequence RXK(R)R of SEQ ID NO: 6.

In some aspects, the polynucleotide further comprises a second p16 promoter.

In some aspects, the first and the second promoter initiate transcription in the same direction.

In some aspects, the first and the second promoter initiate transcription in different directions.

In some aspects, the polynucleotide comprises the nucleic acid sequence of SEQ ID NO: 10.

In some aspects, the polynucleotide comprises a p16 promoter operably linked to a nucleic acid sequence encoding a dominant-negative nuclear factor kappa BIA (dnNKκBIA) protein.

In some aspects, the p16 promoter of said polynucleotide is a human or murine p16 promoter.

In some aspects, the human p16 promoter of said polynucleotide has a nucleotide sequence at least 85%, 86%, 87%, 88%, 89%, 90%, 91%, 92%, 93%, 94%, 95%, 96%, 97%, 98%, 99%, or 100% identical to SEQ ID NO: 1.

In some aspects, the murine p16 promoter of said polynucleotide has a nucleotide sequence at least 85%, 86%, 87%, 88%, 89%, 90%, 91%, 92%, 93%, 94%, 95%, 96%, 97%, 98%, 99%, or 100% identical to SEQ ID NO: 2.

In some aspects, the nucleic acid sequence of said polynucleotide encodes a human dnNKκBIA protein.

In some aspects, the nucleic acid sequence of said polynucleotide encodes the dnNKκBIA protein comprising SEQ ID NO: 7.

In some aspects, said polynucleotide further comprises a WPRE sequence and a polyadenylation signal sequence.

In some aspects, the polyadenylation signal sequence is an SV40 polyadenylation signal sequence, a human growth hormone polyadenylation signal sequence, or a bovine growth hormone polyadenylation signal sequence.

In some aspects, said polynucleotide further comprises an IRES.

In some aspects, said polynucleotide further comprises a proteolytic cleavage site.

In some aspects, the proteolytic cleavage site is selected from the group consisting of a self-processing cleavage site and a furin cleavage site.

In some aspects, the self-processing cleavage site is a P2A, E2A, F2A, or T2A peptide.

In some aspects, the furin cleavage site comprises the consensus sequence RXK(R)R of SEQ ID NO: 6.

In some aspects, a polynucleotide described herein further comprises a reporter gene.

In some aspects, the polynucleotide comprises the nucleic acid sequence of SEQ ID NO: 11.

In some aspects, the polynucleotide comprises the nucleic acid sequence of SEQ ID NO: 12.

In some aspects, provided is a vector comprising a polynucleotide as described herein.

In some aspects, the vector is a viral vector, a non-viral vector, or a polymer.

In some aspects, the viral vector is an adeno-associated viral (AAV) vector, an adenoviral vector, a lentiviral vector, or a retroviral vector.

In some aspects, the AAV vector is an AAV1, AAV2, AAV3, AAV4, AAV5, AAV6, AAV7, AAV8, AAVRH8, AAVrh9, AAV9, AAVrh10, AAV10, AAVRH10, AAV11, AAV12, or AAV-DJ vector.

In some aspects, the AAV vector is an AAV-DJ vector.

In some aspects, the non-viral vector is a plasmid.

In some aspects, the polymer is a cationic polymer comprising a polyethyleneimine (PEI) backbone linked to a lipid or a polyethylene glycol (PEG).

In some aspects, provided is a cell comprising a polynucleotide as described herein or a vector as described herein.

In some aspects, the cell is a bacterial cell, an insect cell, or an animal cell.

In some aspects, the animal cell is a mammalian cell.

In some aspects, provided is a composition comprising the polynucleotide as described herein or the vector as described herein, and a carrier.

In some aspects, provided is a recombinant adeno-associated vector (rAAV) virus comprising the polynucleotide as described herein and an AAV capsid protein.

In some aspects, the rAAV comprises an AAV-DJ capsid.

In some aspects, provided is a pharmaceutical composition comprising the polynucleotide as described herein, the vector as described herein, the cell as described herein, the composition as described herein, or the rAAV as described herein, and a pharmaceutically acceptable carrier.

In some aspects, provided is a kit comprising the polynucleotide as described herein, the vector as described herein, the cell as described herein, the composition as described herein, or the rAAV as described herein.

In some aspects, provided is a method for making a rAAV.

In some aspects, the method comprises providing a cell with adeno-associated virus (AAV) rep genes, AAV cap genes, a nucleic acid comprising the polynucleotide as described herein, the vector as described herein, and at least one AAV serotype 2 inverted terminal repeat.

In some aspects, the method further comprises providing helper functions for generating a productive AAV infection.

In some aspects, the method further comprises allowing assembly of the AAV and collecting the rAAV.

In some aspects, the AAV rep genes and AAV cap genes are provided by a plasmid.

In some aspects, the AAV rep genes and AAV cap genes are stably integrated into the genome of the cell.

In some aspects, the helper functions are provided by a plasmid.

In some aspects, the helper functions are provided by an adenovirus vector.

In some aspects, provided is a rAAV produced by the method described herein.

In some aspects, provided is a method of reprogramming a senescent cell to a non-senescent stage phenotype. In some aspects, the method comprises contacting a senescent cell with the polynucleotide as described herein, the vector as described herein, the cell as described herein, the composition as described herein, the rAAV as described herein, or the pharmaceutical composition as described herein in an amount effective to partially reprogram the senescent cell to a non-senescent stage phenotype.

In some aspects, provided is a method for reprogramming senescent cells in a subject to a non-senescent stage phenotype. In some aspects, the method comprises administering to the subject in need thereof a therapeutically effective amount of the polynucleotide as described herein, the vector as described herein, the cell as described herein, the composition as described herein, the rAAV as described herein, or the pharmaceutical composition as described herein, and partially reprogramming senescent cells of the subject to a non-senescent stage phenotype. In some aspects, the administering is by systemic administration. In some aspects, provided is a method for fully or partially reverting immune senescence in a subject, the method comprising: administering to the subject in need thereof the polynucleotide as described herein, the vector as described herein, the cell as described herein, the composition as described herein, the rAAV as described herein, or the pharmaceutical composition as described herein in an amount effective to fully or partially revert immune senescence in the subject. In some aspects, the administering is by systemic administration.

In some aspects, the immune senescence is characterized by a decrease in a homing ability of a hematopoietic stem cell (HSC) and the amount effective to partially or fully revert immune senescence in the subject is an amount that is effective to increase the homing ability of the HSC to a level of HSC homing ability found in a HSC of a non-senescent subject.

In some aspects, the immune senescence is characterized by a decrease in per-cell repopulating self-renewal activity of a HSC and the amount effective to partially or fully revert immune senescence in the subject is an amount effective to increase the per-cell repopulating self-renewal activity of the HSC to a level of HSC per-cell repopulating self-renewal activity found in a HSC of a non-senescent subject.

In some aspects, the immune senescence is characterized by a bias towards myeloid differentiation of a HSC and the amount effective to partially or fully revert immune senescence in the subject is an amount effective to reduce the bias towards myeloid differentiation of the HSC to a level of bias towards myeloid differentiation found in a HSC of a non-senescent subject.

In some aspects, the immune senescence is characterized by an increase in apoptosis of a HSC upon stress stimuli and the amount effective to partially or fully revert immune senescence in the subject is an amount effective to reduce apoptosis of the HSC upon stress stimuli to a level of apoptosis upon stress stimuli found in a HSC of a non-senescent subject.

In some aspects, provided is a method for reducing the number of senescent cells in a subject, the method comprising: administering to the subject in need thereof the polynucleotide as described herein, the vector as described herein, the cell as described herein, the composition as described herein, the rAAV as described herein, or the pharmaceutical composition as described herein in an amount effective to reduce the number of senescent cells in the subject. In some aspects, the administering is by systemic administration.

In some aspects, provided is a method for extending organismal lifespan of a subject, the method comprising: administering to the subject in need thereof the polynucleotide as described herein, the vector as described herein, the cell as described herein, the composition as described herein, the rAAV as described herein, or the pharmaceutical composition as described herein in an amount effective to extend organismal lifespan of the subject. In some aspects, the administering is by systemic administration.

In some aspects, provided is a method for reducing progressive body weight loss in a senescent subject, the method comprising: administering to the senescent subject in need thereof the polynucleotide as described herein, the vector as described herein, the cell as described herein, the composition as described herein, the rAAV as described herein, or the pharmaceutical composition as described herein in an amount effective to reduce progressive body weight loss in the senescent subject. In some aspects, the administering is by systemic administration.

In some aspects, provided is a method for increasing physical fitness in a senescent subject, the method comprising: administering to the senescent subject in need thereof the polynucleotide as described herein, the vector as described herein, the cell as described herein, the composition as described herein, the rAAV as described herein, or the pharmaceutical composition as described herein in an amount effective to increase physical fitness in the senescent subject. In some aspects, the administering is by systemic administration.

In some aspects, provided is a method for reducing an inflammatory response in a subject, the method comprising: administering to the subject in need thereof the polynucleotide as described herein, the vector as described herein, the cell as described herein, the composition as described herein, the rAAV as described herein, or the pharmaceutical composition as described herein in an amount effective to reduce an inflammatory response in the subject.

In some aspects, provided is a method for reducing replicative senescence in a subject, the method comprising: administering to the subject in need thereof the polynucleotide as described herein, the vector as described herein, the cell as described herein, the composition as described herein, the rAAV as described herein, or the pharmaceutical composition as described herein in an amount effective to reduce replicative senescence in the subject. In some aspects, the administering is by systemic administration.

In some aspects, provided is a method for reducing DNA damage induced senescence in a subject, the method comprising: administering to the subject in need thereof the polynucleotide as described herein, the vector as described herein, the cell as described herein, the composition as described herein, the rAAV as described herein, or the pharmaceutical composition as described herein in an amount effective to reduce DNA damage induced senescence in the subject. In some aspects, the administering is by systemic administration.

In some aspects, provided is a method of reducing oncogene induced senescence in a subject, the method comprising: administering to the subject in need thereof the polynucleotide as described herein, the vector as described herein, the cell as described herein, the composition as described herein, the rAAV as described herein, or the pharmaceutical composition as described herein in an amount effective to reduce oncogene induced senescence in the subject. In some aspects, the administering is by systemic administration.

In some aspects, provided is a method for inhibiting Senescence-Associated Secretory Phenotype (SASP) activity in a cell. In some aspects, the method comprises contacting the cell with the polynucleotide as described herein, the vector as described herein, the cell as described herein, the composition as described herein, the rAAV as described herein, or the pharmaceutical composition as described herein, in an amount effective to inhibit SASP activity in the cell.

In some aspects, provided is a method for rejuvenating a senescent cell to a non-senescent stage phenotype. In some aspects, the method comprises contacting a senescent cell with the polynucleotide as described herein, the vector as described herein, the cell as described herein, the composition as described herein, the rAAV as described herein, or the pharmaceutical composition as described herein in an amount effective to partially reprogram the senescent cell. In some aspects, the method further comprises contacting the senescent cell the polynucleotide as described herein, or the vector as described herein in an amount effective to inhibit SASP activity in the senescent cell.

In some aspects, provided is a method for rejuvenating senescent cells in a subject to a non-senescent stage phenotype. In some aspects, the method comprises administering to a subject in need thereof a therapeutically effective amount of the polynucleotide as described herein, the vector as described herein, the cell as described herein, the composition as described herein, the rAAV as described herein, or the pharmaceutical composition as described herein, to partially reprogram the senescent cells; and administering to the subject a therapeutically effective amount of the polynucleotide as described herein, or the vector as described herein, to inhibit SASP activity. In some aspects, the administering is by systemic administration.

In some aspects, provided is a method for increasing age-related survival of a subject. In some aspects, the method comprises administering to a subject in need thereof a therapeutically effective amount of the polynucleotide as described herein, the vector as described herein, the cell as described herein, the composition as described herein, the rAAV as described herein, or the pharmaceutical composition as described herein, and reprogramming senescent cells in the subject, whereby the age-related survival of the subject is increased. In some aspects, the administering is by systemic administration.

In some aspects, provided is a method for treating cellular senescence-related aging in a subject in need thereof. In some aspects, the method comprises administering to subject a therapeutically effective amount of the polynucleotide as described herein, the vector as described herein, the cell as described herein, the composition as described herein, the rAAV as described herein, or the pharmaceutical composition as described herein. In some aspects, the administering is by systemic administration.

In some aspects, provided is a method of treating Hutchinson-Gilford Progeria Syndrome in a subject in need thereof. In some aspects, the method comprises administering to subject a therapeutically effective amount of the polynucleotide as described herein, the vector as described herein, the cell as described herein, the composition as described herein, the rAAV as described herein, or the pharmaceutical composition as described herein. In some aspects, the administering is by systemic administration.

In some aspects, the subject is a human.

In some aspects, the administration induces increased epidermal thickness, reduced mesangial area in the kidney, increased structural arrangement of hepatocytes in the liver, rescued lymphoid depletion in germinal centers of the spleen, reduced level of inflammatory markers, increased number of hematopoietic stem cells, restoration of bone marrow tissue and/or an increased activation of DNA repair pathways in the subject.

In some aspects, the reduced level of inflammatory markers comprises reduced neutrophil invasion in the liver and a reduced inflammatory signature in the spleen.

In some aspects, a method of treatment is provided, the method comprising contacting a cell with a polynucleotide as described herein, a vector as described herein, a cell as described herein, a composition as described herein, a rAAV as described herein, or a pharmaceutical composition as described herein in an amount effective to partially reprogram the cell, In some embodiments, the method further comprises administering the cell to a subject in need of a partially reprogrammed cell. In some aspects, the cell is a fibroblast. In some aspects, the fibroblast is a mammalian fibroblast. In some aspects, the fibroblast is a human fibroblast.

In some aspects, provided is a use of the polynucleotide as described herein, the vector as described herein, the cell as described herein, the composition as described herein, the rAAV as described herein, or the pharmaceutical composition as described herein for treating cellular senescence-related aging.

In some aspects, provided is a use of the polynucleotide as described herein, the vector as described herein, the cell as described herein, the composition as described herein, the rAAV as described herein, or the pharmaceutical composition as described herein in the manufacture of a medicament for treating cellular senescence-related aging.

In some aspects, provided is an article of manufacture comprising the polynucleotide as described herein, the vector as described herein, the cell as described herein, the composition as described herein, the rAAV as described herein, or the pharmaceutical composition as described herein.

### BRIEF DESCRIPTION OF THE FIGURES

FIGS. 1A-1H. Senescence cell specific cellular reprogramming improves health and life span in LAKI^{-/-} mice. FIG. 1A: Graphical representation of constructs for expression of mOct4, mSox2, mKlf4 (OSK) or human dominant negative NFκBIA and GFP. All constructs are in an AAV plasmid backbone. FIG. 1B: Graphical representation of mice experiments to evaluate the effect of CDKN2Apromoter-OSK and CDKN2Apromoter-NFKBIA expression in mouse health and life span. FIG. 1C: Survival curves of LAKI^{-/-} mice injected with control CAG-GFP (green line), m-CDKN2A promoter-OSK (blue line), or m-CDKN2A promoter-NFKBIA (dn) (red line) AAV DJ particles compared to untreated mice (grey line). (GFP, n=15; m-CDKN2A promoter-OSK n=18; m-CDKN2A promoter-NFκBIA (dn) n=18, no treatment n=18). FIG. 1D: Histological analyses of the skin and spleen derived from 135 days old wild-type mice or LAKI^{-/-} mice transduced with CAG-GFP or m-CDKN2A promoter-OSK AAV at day 60 as depicted in FIG. 1B; the yellow and white arrows in the spleen section show splenic nodules and red pulp respectively; scale bar represents 100 μm. FIG. 1E: Frequency of pluripotent hematopoietic stem cells (pHSC), lymphoid primed multipotent progenitors (LMPP), and multipotent progenitors (MPP) in the hematopoietic stem cell multipotent progenitor compartment (HSC-MMP) in mice upon m-CDKN2A promoter-OSK expression compared to CAG-GFP expression. FIG. 1F: Frequency of HSC compartment subpopulations in mice upon m-CDKN2A promoter-OSK expression compared to CAG-GFP expression. FIG. 1G: RT-PCR measurements of levels of CDKN2A, and key inflammatory marker genes in various organs of mice treated with CAG-GFP or m-CDKN2A promoter-OSK; mRNA levels are shown relative to Gapdh. FIG. 1H: GSEA analysis of genes that were significantly de-regulated upon CDKN2A promoter-OSK induction in skin, liver, spleen and bone marrow, respectively.

FIGS. 2A-2F. Optimization of senescence cell specific rejuvenation strategy. FIG. 2A: Single cell RNA sequencing analyses of multiple human organs derived from different individuals of various age groups reveal four main cell clusters: epithelial, endothelial, stromal, and immune cell clusters (left); high CDKN2A expression color coded based on individual age (middle) and the identity of high CDKN2A expressing cells (right) are shown. Data were derived from the Tabula Sapiens consortium. FIG. 2B: Graphical representation of NFκBIA protein domains and critical serine residues for post translational regulation. Amino acid sequences of wild-type and dominant negative isoforms for NFκBIA, where two critical serine residues were replaced by alanine residues. FIG. 2C: Representative immunofluorescence image showing localization of NFKB and actin proteins in healthy and senescence cells (3T3 mouse fibroblast) transfected with AAV harboring either GFP or a dominant negative isoform of NFKBIA. FIG. 2D: Schematic representation of the cell culture experiments in mouse embryonic fibroblasts (MEFs) to test specificity of AAV mediated CDKN2A promoter driven expression of target genes during senescence induced via the DNA damaging agent Etoposide (top); RT-PCR measurement of levels of CDKN2A, Oct4 and Sox2 during senescence. mRNA levels are shown relative to a TATA-binding protein (Tbp) (bottom). FIG. 2E: Graphical representation of mouse experiments in CDKN2A-3MR mice to evaluate organs receiving AAV-DJ harboring a m-CDKN2A-GFP transgene. FIG. 2F: Representative immunofluorescence images showing expression of AAV m-CDKN2A promoter-GFP in p16-3MR mice in key organs including spleen, liver, and skin. p16-3MR mice consist of a trimodal reporter constituted of functional domains for LUC (Renilla Luciferase), mRFP (monomeric red fluorescent protein), and HSV-TK (truncated herpes simplex virus 1 thymidine kinase under control of p16-INK4a and p19-Arf promoter. Scale bars represent 100 or 20 μm.

FIGS. 3A-3G. AAV mediated senescence cell specific transgene expression. FIG. 3A: Graphical representation of experiments to evaluate AAV-DJ organ tropism and retention over time in young and old mice. FIG. 3B: RT-qPCR measurement of levels of AAV viral particles with primers specific for the WPRE region of the recombinant AAV genome in DNA isolated from different organs. DNA amount normalized to a mouse intergenic region. y-axis represent relative AAV genomic DNA.

FIG. 3C: Relative level of p16 expression in various organs measured by RT-qPCR in various organs of 4.5 months old LAKI−/− and wild-type B6 mice. mRNA levels shown on y-axis are relative to Gapdh level. FIG. 3D: Graphical representation of mice experiments to evaluate AAV-DJ mediated transgene expression in LAKI−/− mice FIG. 3E: RT-qPCR measurement of levels of Oct4 in various organs of LAKI−/− mice injected with AAV DJ Control-GFP or CDKN2A promoter-OSK. mRNA levels are shown relative to Gapdh level. FIG. 3F: Genome browser tracks derived from ChIP assay for H3K27 acetylation mark performed in proliferative and senescence IMR90 cells show its enrichment at the promoter of CDKN2A and MIR146A gene. FIG. 3G: Survival curve of LAKI−/− mice injected with control-GFP (green line) or CDKN2A promoter-OSK (blue line) or mir146-OSK (magenta line) harboring AAV DJ particles. The x-axis represents days, and the y-axis represents % survival.

FIG. 4A-4G. Improvement in health span upon senescence cell specific OSK expression. FIG. 4A: Body weight of LAKI^{-/-} mice at various time points upon one injection of control CAG-GFP or m-CDKN2A promoter-OSK AAV-DJ particles. FIG. 4B: Bar plots showing quantification of various activities based on the Open Field Maze experiments in control CAG-GFP and m-CDKN2A promoter-OSK injected LAKI^{-/-} mice at day 135. FIG. 4C: Necropsy analysis of 135 days old LAKI^{-/-} mice injected with control CAG-GFP or m-CDKN2A promoter-OSK AAV-DJ particles. White arrows indicate megacolon/megacecum, an abnormal gastrointestinal phenotype observed in control CAG-GFP but recovered in m-CDKN2A promoter-OSK injected LAKI^{-/-} mice. FIG. 4D: Images of spleen at day 135 of LAKI^{-/-} mice injected with control CAG-GFP or m-CDKN2A promoter-OSK AAV-DJ particles. FIG. 4E: Histological analyses of kidneys derived from 135 days old wild-type mice or LAKI−/− mice transduced with CAG-GFP or m-CDKN2A promoter-OSK AAV-DJ particles at day 60 as shown in FIG. 1C. FIG. 4F: Histological analyses of kidney and liver sections derived from 135 days old wild-type mice or LAKI−/− mice transduced with CAG-GFP or m-CDKN2A promoter-OSK AAV at day 60 as shown in FIG. 1C; the yellow arrows in the kidney sections show the glomerular capsule; the green arrows in the kidney sections show the mesangial area of the glomerular capsule; scale bar represents 100 or 20 μm. FIG. 4G: Bar plot showing RNA sequencing data for expression levels of Oct4, Sox2 and Klf4 in various organs upon CDKN2A promoter-OSK.

FIG. 5A-5I. Bone marrow compartments rejuvenated towards the young stage upon CDKN2A promoter-OSK induction. FIG. 5A: Gating strategies and representative plots for young and old wild-type mice bone marrow compartment used for characterization of hematopoietic stem cells (HSC) and various progenitors; areas delineated by the black boxes in the scatter plots indicate the population of cells gated; lineage markers (Lin) included cocktails of antibodies recognizing CD3ε, B220, CD11b, Ly-6G and TER-119. FIG. 5B: CD135 and CD34 markers were used on KSL population (Kit (+), Sca-1 (+) and Lin (−)) to characterize the HSC-MMP compartment in young and old wild-type mice. CD150 and CD41 markers were used on KSL CD34 (−) population to characterize the HSC compartment. MPP: multipotent progenitor; LMPP: lymphoid-primed multipotent progenitors; pHSC: pluripotent hematopoietic stem cells. FIG. 5C: Representative flow cytometric data of the hematopoietic stem cell compartment in bone marrow from LAKI^{-/-} young and LAKI^{-/-} old mice injected with either control CAG-GFP or CDKN2A promoter-OSK AAV DJ, showing distribution of cells in the HSC-MMP compartment; areas delineated by the black boxes in the scatter plots indicate the respective MPP, LMPP, and pHSC populations gated. FIG. 5D: Representative flow cytometric data of bone marrow from LAKI−/− young and LAKI−/− old mice injected with either control GFP or CDKN2A promoter-OSK AAV DJ, showing distribution of cells in the HSC compartment. FIG. 5E: Representative immunofluorescence image of bone marrow from LAKI−/− old mice injected with AAV-DJ harboring either control-GFP or CDKN2A promoter-OSK, showing abundance of Ki67, Hes1 and CD45 positive cells. FIG. 5F: Graphical representation of in vitro expansion of SLAM (signaling lymphocyte activation molecule) HSCs from LAKI−/− mice. Fifty E-SLAM (EPCR, endothelial protein C receptor) HSCs from bone marrow were sorted into each well of 96 well plate with differentiation medium. Twelve days post culture, cell growth and differentiation were observed by microscopy and FACS analysis. FIG. 5G: Representative images showing various arbitrary groups of expanded HSC colonies after 12 days in differentiation medium.

FIG. 5I: Stacked bar plot showing distribution of HSC colony size in CDKN2A promoter-OSK treated and control HSC. FIG. 5I: Frequency of the HSC-MMP subpopulations of cultured cells of LAKI−/− mice treated with CDKN2A promoter-OSK compared to control. Dots represent individual set of experiment derived from independent mice, and horizontal lines indicate SEM.

FIG. 6A-6E. Organ specific transcriptional changes upon CDKN2A promoter-OSK induction. FIG. 6A: Volcano plot representation of RNA-seq results shows the number of genes with a significantly altered expression in skin upon AAV DJ CDKN2A promoter-OSK in comparison to control mice injected with AAV-DJ CAG-GFP. Tissues were collected at day 135, from the same LAKI^{-/-} mice used in FIG. 1B. On the right hand side of the volcano plot, GSEA of genes that were significantly de-regulated upon AAV DJ CDKN2A promoter-OSK in the skin. FIG. 6B: Volcano plot representation of RNA-seq results shows the number of genes with a significantly altered expression in the liver upon AAV DJ CDKN2A promoter-OSK in comparison to control mice injected with AAV-DJ CAG-GFP and GSEA of genes that were significantly de-regulated upon AAV DJ CDKN2A promoter-OSK in the liver. FIG. 6C: Volcano plot representation of RNA-seq results shows the number of genes with a significantly altered expression in the spleen upon AAV DJ CDKN2A promoter-OSK in comparison to control mice injected with AAV-DJ CAG-GFP and GSEA of genes that were significantly de-regulated upon AAV DJ CDKN2A promoter-OSK in the spleen. FIG. 6D: Volcano plot representation of RNA-seq results shows the number of genes with a significantly altered expression in the bone marrow upon AAV DJ CDKN2A promoter-OSK in comparison to control mice injected with AAV-DJ CAG-GFP and GSEA of genes that were significantly de-regulated upon AAV DJ CDKN2A promoter-OSK in the bone marrow. FIG. 6E: Bar plot showing expression level of set of tissue specific marker genes in various organs (skin, liver, spleen and bone marrow) upon AAV DJ CDKN2A promoter-OSK. y-axis represent normalized reads.

FIG. 7A-7F. Transcriptional rejuvenation in human primary cells (IMR90). FIG. 7A: Bar plots showing mRNA levels for CDKN2A promoter, and pluripotent factors Oct4, Sox2 and Klf4 in IMR90 cells transduced with AAV harboring CDKN2A promoter-OSK at various stages of replicative senescence. Day 20 represents healthy young cells, day 125 represents the complete senescence stage, while day 50 and day 85 represent intermediate stages. y-axis represents normalized tag counts. FIG. 7B: Principal component analysis (PCA) plot of control- and CDKN2A promoter-OSK-treated IMR90 cells at various stages of replicative senescence. FIG. 7C: Heat map showing the expression of inflammation associated genes upon CDKN2A promoter-OSK AAV-DJ during replicative senescence. FIG. 7D: Heat map showing the expression of cell cycle related genes upon CDKN2A promoter-OSK AAV-DJ during replicative senescence. FIG. 7E: Heat map showing the expression of inflammation associated genes upon CDKN2A promoter-OSK during DNA damage induced (via treatment of Etoposide or Bleomycin) and oncogenic RAS induced senescence. FIG. 7F: Bioluminescence images showing abundance of senescence cells in 24 months old CDKN2A promoter-3MR mice, two months post injection of a single dose of either control or CDKN2A promoter-OSK AAV particles.

FIG. 8A-8F. CDKN2A promoter-OSK rejuvenate senescent human fibroblasts. FIG. 8A: Representative brightfield images of IMR90 cells upon 12 days post AAV DJ human CDKN2A promoter human OSK (H-CDKN2Ap-hOSK) treatment. FIG. 8B: The gating strategies and representative plot for apoptosis analysis of young and senescent human fibroblast; the areas delineated by the black boxes in the scatter plots indicate the population of cells gated. FIG. 8C: The gating strategies and representative plot for cell cycle analysis of young and senescent human fibroblast; the areas delineated by the black ovals in the scatter plots indicate the population of cells gated. FIG. 8D: Bar plot showing frequency of cells in various phases of cell cycle upon H-CDKN2Ap-hOSK AAV-DJ in IMR90 cells. y-axis represent % of cells. FIG. 8E: Bar plot showing expression levels of fibroblast specific marker genes in IMR90 upon H-CDKN2Ap-hOSK AAV-DJ. y-axis represent normalized reads. FIG. 8F: Bar plot showing mRNA levels of senescence associated genes IFIH1 and OAS2 upon H-CDKN2Ap-hOSK AAV-DJ in IMR90 cells. y-axis represent normalized read counts.

FIG. 9A-9I. Transcriptional rejuvenation upon CDKN2A promoter-OSK in human fibroblasts. FIG. 9A: KEGG pathway enrichment analysis of deregulated genes during senescence. FIG. 9B: KEGG pathway enrichment analysis of deregulated genes upon H-CDKN2A promoter-hOSK. FIG. 9C: Box plot showing expression levels of IFN responsive and cell cycle related genes upon H-CDKN2A promoter-hOSK during replicative senescence in IMR90 cells. y-axis represents normalized reads. FIG. 9D: Bar plot showing mRNA levels of ECM associated genes Elastin and COL1A1 upon H-CDKN2A promoter-hOSK in IMR90 cells. y-axis represents normalized read counts. FIG. 9E: Heat map showing the expression of extracellular matrix (ECM) associated genes upon H-CDKN2A promoter-OSK during replicative senescence. FIG. 9F: Bar plot showing RNA levels of CDKN2A, POU5F1 (Oct4), SOX2, and KLF4 in IMR90 cells untreated or treated with Belomycin, Etoposide, or Oncogenic RAS. FIG. 9G: Principal component analysis (PCA) plot of control- and H-CDKN2A promoter-hOSK-treated IMR90 cells that have undergone DNA damage (via treatment of Etoposide or Bleomycin) or oncogene induced senescence by RAS. FIG. 9H: Graphical representation of p16-3MR mouse experiments to evaluate the effect of H-CDKN2A promoter-OSK on senescence cell populations.

FIG. 91: Bioluminescence images showing abundance of senescence cells in 26 months old p16 (CDKN2A) promoter-3MR mice, four months post injection of a single dose of either control or m-CDKN2A promoter-OSK AAV particles.

FIG. 10A-10C. Rejuvenation of wild-type mice upon senescence cell specific reprogramming. FIG. 10A: Graphical representation of mouse experiments to evaluate the long-term effects of m-CDKN2A (p16) promoter-OSK expression in young wild-type mice (top) and survival curve of mice injected with control m-CDKN2A (p16) promoter-GFP (green line) or m-CDKN2A (p16) promoter-OSK (red line) AAV-DJ particles (bottom). GFP, (n=6); m-CDKN2A promoter-OSK (n=6). The x-axis represents days, and the y-axis represents % survival. FIG. 10B: Graphical representation of mouse experiments to evaluate the effects of m-CDKN2A (p16) promoter-OSK on health and life span in wild-type physiologically old mice (top); body weight of mice at various time points upon a single injection of AAV-DJ harboring a control m-CDKN2A (p16) promoter-GFP or a m-CDKN2A (p16) promoter-OSK transgene (bottom). The y-axis represents total body weight in grams and the x-axis represents age in days. FIG. 10C: Survival curves of wild-type mice injected with control m-CDKN2A promoter-GFP (green line) or m-CDKN2A promoter-OSK (red line) AAV-DJ particles. (GFP, n=20; m-CDKN2A promoter-OSK, n=24). The x-axis represents days, and the y-axis represents % survival.

FIG. 11A-11B. Senescence cell specific reprogramming improves resilience in wild-type mice. FIG. 11A: Tabular representation of tumor frequency in control and m-CDKN2A promoter-OSK mice used in different experiments. Evidence of a tumor was established during necropsy. FIG. 11B: Bar plots showing quantification of various activities based on treadmill tests in control and m-CDKN2A promoter-OSK AAV-DJ injected old wild-type mice.

### DETAILED DESCRIPTION

Provided are materials and methods for rejuvenating senescent cells by expressing rejuvenation factors, e.g., Oct4, Sox2, and Klf4 (OSK) proteins and/or a dominant negative nuclear factor kappa B IA (dnNFκBIA) in senescent cells. In some aspects, the expression of OSK results in partial reprogramming in senescent cells. In some aspects, the expression of OSK results in full or partial reversal of aging in aged cells. In some aspects, the expression of a dnNFκBIA protein results in a suppression of a Senescence-Associated Secretory Phenotype (SASP) in senescent cells. In some aspects, provided are polynucleotides and vectors comprising OSK under the control of a CDKN2A (p16) promoter. The polynucleotides and vectors enable expression of OSK and/or dnNFκBIA specifically in senescent cells for targeted phenotype changes in senescent cells while avoiding systemic changes in non-senescent cells. The polynucleotides and vectors also enable expression of OSK and/or dnNFxBIA specifically in aged and/or aging cells for targeted phenotype changes while avoiding systemic changes in non-aged cells.

Without being bound by theory, applicants believe that expression of reprogramming factors, e.g., OSK, driven by the CDKN2A (p16) promoter decreases or attenuates as the senescent cells are rejuvenated and/or partially reprogrammed. Without being bound by theory, applicants believe that expression of reprogramming factors, e.g., OSK, driven by the CDKN2A (p16) promoter decreases or attenuates aging in aged cells. The aged cells can be naturally aged cells or prematurely aged cells.

Without being bound by theory, expression of reprogramming factors, e.g., OSK, driven by the CDKN2A (p16) promoter should decrease as the senescent cells are rejuvenated and/or partially reprogrammed, such that the OSK expression may serve as a marker of rejuvenation and/or partial reprogramming. Other markers of rejuvenation and/or partial reprogramming such as downregulation of inflammatory/SASP/Stress response/DNA damage/Reactive oxygen species (ROS) genes are disclosed herein.

### I. Definitions

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

The terms “polynucleotide,” “nucleic acid,” and “polynucleotide,” are used interchangeably in the present application. These terms refer only to the primary structure of the molecule. Thus, these terms include double- and single-stranded DNA, as well as double- and single-stranded RNA. The terms “nucleic acid,” “polynucleotide,” and “polynucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

The term “polypeptide,” as used herein, is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with, any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein, encompasses full length peptides and fragments, variants or derivatives thereof. A “peptide” as disclosed herein can be part of a fusion polypeptide comprising additional components such as, e.g., an albumin or PEG moiety, to increase half-life. A peptide as described herein can also be derivatized in a number of different ways. A peptide described herein can comprise modifications including e.g., conjugation of a palmitoyl group.

The term “in vitro,” as used herein, refers to events that occur in an artificial environment, e.g., in a test tube or reaction vessel, in cell culture, in a Petri dish, etc., rather than within an organism (e.g., animal, plant, or microbe).

The term “in vivo,” as used herein, refers to events that occur within an organism (e.g., animal, plant, or microbe or cell or tissue thereof).

The term “transfection,” as used herein, refers to methods to introduce exogenous nucleic acids into a cell. Methods of transfection include, but are not limited to, chemical methods, physical treatments and cationic lipids or mixtures. The list of agents that can be transfected into a cell is large and includes, e.g., siRNA, shRNA, sense and/or anti-sense sequences, DNA encoding one or more genes and organized into an expression plasmid, e.g., a vector.

The term “percent (%) sequence identity,” as used herein, with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

The term “level,” as used herein, refers to an amount or activity of a protein, or mRNA encoding the protein, optionally as compared to a reference. The reference can be any useful reference, as defined herein. By a “decreased level” or an “increased level” of a protein or RNA is meant a decrease or increase in protein or RNA level, as compared to a reference. A level of a protein or RNA can be expressed in mass/vol (e.g., g/dL, mg/mL, μg/mL, ng/mL) or percentage relative to total protein or RNA in a sample.

The term “promoter,” as used herein, refers to a DNA sequence recognized by the machinery of a cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible expression by external signals or agents; such elements can be located in the 5′ or 3′ regions of the native gene. In some aspects, the promoter can be a constitutively active promoter, a cell-type specific promoter, or an inducible promoter.

The term “senescent-cell specific promoter,” as used herein, refers to a promoter that is expressed in a senescent cell but not, or in a significantly reduced manner, in a non-senescent cell. Examples of senescent-cell specific promoters include, but are not limited to, a CDKN2A promoter (also termed p16 promoter), or a mir146a promoter. The terms p16 promoter and CDKN2A promoter are used interchangeably herein.

The term “CDKN2A promoter” or “p16 promoter,” as used herein, refers to the promoter of the human or mouse cyclin-dependent kinase inhibitor 2A (CDKN2A) gene. The human CDKN2A (p16) promoter is provided in SEQ ID NO: 1. The mouse CDKN2A (p16) promoter is provided in SEQ ID NO: 2.

The term “WPRE,” as used herein, refers to a Woodchuck Hepatitis Virus Post-translational Regulatory Element that is a DNA sequence that, when transcribed, creates a tertiary structure enhancing expression of genes of a viral vector.

The term “IRES,” as used herein, refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). Under translational control of an IRES translation proceeds in a cap-independent manner.

The terms “operatively linked,” “operatively inserted,” “operatively positioned,” “under control” or “under transcriptional control” mean that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s). The term “operably inserted” means that the DNA of interest introduced into the cell is positioned adjacent a DNA sequence which directs transcription and translation of the introduced DNA (i.e., facilitates the production of, e.g., a polypeptide encoded by a DNA of interest).

The term “proteolytic cleavable site,” as used herein, refers to a polynucleotide encoding an amino acid sequence that can be proteolytically cleaved and includes, but is not limited to, a self-processing cleavage site and a furin cleavage site.

The term “self-processing cleavage site,” as used herein, refers to a post-translational or co-translational processing cleavage site or sequence that can be a DNA or amino acid sequence, exemplified herein by a 2A site, sequence or domain or a 2A-like site, sequence or domain. The self-processing peptide is the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

The term “furin cleavage site,” as used herein, refers to a nucleic acid encoding an amino acid sequence that can be cleaved by endogenous subtilisin-like proteases, such as furin and other serine proteases within the protein secretion pathway. In some aspects, the furin cleavage site includes a consensus sequence RXK(R)R of SEQ ID NO:6.

The terms “dominant-negative nuclear factor kappa BIA” or “dnNFκBIA,” as used herein, refer to a dominant negative isoform of NFκBIA that is efficient in blocking nuclear migration of NFκB, a master transcriptional inducer of SASP.

The term “termination signal sequence,” as used herein, can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

The term “LAK^{-/-} mouse,” as used herein, refers to a premature aging mouse model with a point mutation in the Lmn gene, which mouse model manifests symptoms reminiscent of accelerated aging in Hutchinson-Gilford Progeria (HGPS) patients.

The term “aged cell,” as used herein refers to a cell in which complex processes associated with genomic and epigenomic alterations occur that eventually result in permanent growth arrest of the cell.

The term “senescence,” as used herein refers to the gradual deterioration of functional characteristics of a cell or organism.

The term “cellular senescence,” as used herein refers to a permanent growth arrest phenotype and chronic DNA damage signaling of a cell; both the permanent growth arrest and chronic DNA damage signaling can drive, among others, the expression and secretion of several cytokines, chemokines, growth factors, and proteases; genomic instability; telomeric attrition; epigenetic alterations; and mitochondrial dysfunction.

The term “senescence-associated secretory phenotype (SASP),” as used herein refers to the expression and secretion of cytokines, chemokines, growth factors, and proteases by senescent cells, which can lead, among others, to a deterioration of the functional competence of stem cells thereby compromising their role in tissue renewal.

The term “organismal senescence,” as used herein refers to the aging of a whole organism and includes a decline in the ability to respond to stress, decrease in rejuvenation capacity, stem cell exhaustion, increase in homeostatic imbalance, loss of proteostasis, and increase in age-associated diseases.

The term “immune senescence,” as used herein refers to an age-related immune dysfunction that includes, among others, thymic involution, an increase in the number of memory T cells, a loss of adaptive diversity, a loss of the ability to respond to antigen and a lingering level of low-grade inflammation.

The term “replicative senescence,” as used herein refers to cellular senescence induced by excessive cell replication.

The term “DNA damage induced senescence,” as used herein refers to senescence induced by DNA damage.

The term “oncogene induced senescence,” as used herein refers to senescence induced by oncogenic signaling resulting from an activating mutation of an oncogene, or the inactivation of a tumor-suppressor gene. The oncogene, for example, can be the Ras GTPase (RAS).

The term “vector,” as used herein, refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., is capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. In some aspects, insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, the delivery vector is selected from the group consisting of a viral vector (e.g., an AAV vector), a plasmid, a lipid, a cationic polymer, a protein particle, a bacterial vector, and a lysosome. Some aspects of the disclosure are directed to biological vectors, which can include viruses, particularly attenuated and/or replication-deficient viruses. In some aspects, a vector can include microRNA targeting sequences to increase specificity of vector-mediated transgene expression. In some aspects, the microRNA targeting sequences are incorporated into a 3′ UTR of the polynucleotide or vector. In some aspects, the delivery vector of the disclosure is a viral vector selected from the group consisting of an adeno-associated viral (AAV) vector, an adenoviral vector, a lentiviral vector, or a retroviral vector.

The terms “adeno-associated virus vector” or “AAV vector,” as used herein, refer to any vector that comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV-DJ, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome” and “AAV vector” can be used interchangeably. In some aspects, the AAV vector is modified relative to the wild-type AAV serotype sequence.

The term “AAV particle,” as used herein, refers to an AAV virus that comprises an AAV vector having at least one payload region (e.g., a polynucleotide encoding a therapeutic protein or peptide) and at least one inverted terminal repeat (ITR) region. In some aspects, the term “AAV vectors” refers to AAV vectors comprising a polynucleotide encoding an Oct4 protein, Sox2 protein, and/or Klf4 protein and/or a dnNFκBIA protein.

The term “AAV rep gene,” as used herein, refers to the large open reading frame (ORF), known as the AAV replication (rep) region, of an AAV genome. This ORF encodes replication gene products Rep78, Rep 68, Rep 52, and Rep 40, which are named for their apparent molecular weights and which allow for the replication, assembly, and packaging of a complete AAV virions.

The term “AAV cap gene,” as used herein, refers to the large open reading frame (ORF), known as the AAV capsid (cap) region, of an AAV genome. This ORF encodes at least three capsid proteins: VP1, VP2, and VP3, which allow for the assembly of AAV capsids into which AAV genomes are packaged with the aid of AAV Rep proteins to generate AAV viruses.

The phrase “AAV helper functions for generating a productive AAV infection,” as used herein, refers to AAV rep and AAV cap genes provided by a source other than the nucleic acid comprising the payload region and the at least one ITR, such that AAV capsid production, AAV payload/ITR nucleic acid replication and AAV payload/ITR insertion into the assembled AAV capsid can occur. AAV helper functions can be provided by co-infection of the AAV producer cells with wild-type AAV virions, by providing one or more plasmids comprising the AAV rep and AAV cap genes to the AAV producer cell, or by infecting the AAV producer cell with a non-AAV virus carrying the AAV rep and AAV cap genes. AAV particles produced according to the methods described herein lack AAV rep and AAV cap genes and contain the AAV payload/ITR nucleic acid. When administered to a cell of a subject, the AAV virion, in the absence of AAV rep and AAV cap genes, cannot replicate or form more AAV virions in the subject's cell. Instead, the AAV virions release their pay load/ITR nucleic acid upon entry into a subject's cells and the payload gene is transcribed in the subject's cell to produce the payload protein.

The phrase “contacting a cell” (e.g., contacting a cell with an AAV vector, an AAV capsid, or the pharmaceutical composition of the disclosure) as used herein, includes contacting a cell directly or indirectly. In some aspects, contacting a cell with an AAV vector, an AAV capsid, or a composition includes contacting a cell in vitro with the composition, the AAV vector, or the AAV capsid or contacting a cell in vivo with the AAV vector, the AAV capsid, or composition. Thus, for example, the AAV vector, AAV capsid, or composition can be put into physical contact with the cell by the individual performing the method, or alternatively, the AAV vector, AAV capsid, or composition can be put into a situation that will permit or cause it to subsequently come into contact with the cell. In some aspects, contacting a cell in vitro can be done, e.g., by incubating the cell with the AAV vector, AAV capsid, or composition. In some aspects, contacting a cell in vivo can be done, e.g., by injecting the AAV vector, AAV capsid, or composition of the disclosure into or near the tissue where a target cell is located, or by injecting the AAV vector, AAV capsid, or composition into an area, e.g., the bloodstream or the subcutaneous space, such that the agent will subsequently reach the tissue where the cell to be contacted is located. Further, an AAV vector or AAV virus can be encapsulated and/or coupled to a ligand that directs the AAV vector or AVA virus to a site of interest. Combinations of in vitro and in vivo methods of contacting are also possible. For example, a cell can be contacted in vitro with an AAV vector, an AAV capsid, or a composition and subsequently transplanted into a subject.

In some aspects, contacting a cell with an AAV vector, an AAV capsid, or a composition of the present disclosure includes “introducing” or “delivering” (directly or indirectly) the AAV vector, the AAV capsid, or the composition into the cell by facilitating or effecting uptake or absorption into the cell. Introducing an AAV vector, an AAV capsid, or a composition into a cell can be in vitro and/or in vivo. For example, for in vivo introduction, an AAV vector, an AAV capsid, a composition can be injected into a specific tissue site (e.g., the locus where a therapeutic effect is desired) or administered systemically (e.g., administering an AAV vector targeted to a locus where a therapeutic effect is desired). In vitro introduction of an AAV genome or a payload/ITR polynucleotide into a cell includes methods known in the art such as electroporation and lipofection.

The terms “effective amount,” “therapeutically effective amount,” and a “sufficient amount” of, e.g., an AAV vector, an AAV capsid, or a composition disclosed herein refer to a quantity sufficient to, when administered to the subject, including a human, effect beneficial or desired results, including clinical results, and, as such, an “effective amount” or synonym thereto depends on the context in which it is being applied. In some aspects, a therapeutically effective amount of an agent (e.g., an AAV vector, an AAV capsid, a composition disclosed herein) is an amount that results in a beneficial or desired result in a subject as compared to a control. The amount of a given agent (e.g., an AAV vector, an AAV capsid, or a composition disclosed herein) will vary depending upon various factors, such as the given agent, the pharmaceutical formulation, the route of administration, the type of disease or disorder, the identity of the subject (e.g., age, sex, and/or weight) or host being treated, and the like.

The term “gene therapy,” as used herein, refers to the insertion of nucleic acid sequences (e.g., a polynucleotide comprising a promoter operably linked to a nucleic acid encoding a therapeutic molecule as disclosed herein) into an individual's cells and/or tissues to treat, reduce the symptoms of, or reduce the likelihood of a disease or aging-related condition. Gene therapy also includes insertion of transgene that are inhibitory in nature, i.e., that inhibit, decrease or reduce expression, activity or function of an endogenous gene or protein, such as an undesirable (e.g., senescence inducing) or aberrant (e.g., pathogenic) gene or protein. Such transgenes can be exogenous. An exogenous molecule or sequence is understood to be molecule or sequence not normally occurring in the cell, tissue and/or individual to be treated.

The term “pharmaceutical composition,” as used herein, represents a composition comprising a compound or molecule described herein, e.g., an AAV vector disclosed herein, formulated with a pharmaceutically acceptable excipient, and can be manufactured or sold with the approval of a governmental regulatory agency as part of a therapeutic regimen for the treatment of a disease in a mammal.

The term “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient.

The term “subject,” as used herein, refers to any organism to which a composition disclosed herein, e.g., an AAV vector of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

The terms “treat,” “treated,” and “treating,” as used herein mean both therapeutic treatment and prophylactic or preventative measures wherein the object is to prevent or slow down (lessen) an undesired physiological, e.g., an age-related, condition, disorder, or disease, or obtain beneficial or desired clinical results. In some aspects, an age-related condition includes signs and/or symptoms associated with natural aging. In some aspects, treating reduces or lessens the symptoms associated with, e.g., an age-related disease or disorder. In some aspects, the treating results in a beneficial or desired clinical result. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. Ameliorating the disease or disorder includes slowing the course of the disease or disorder or reducing the severity of later-developing an age-related disease or disorder. The “prophylactically effective amount” can vary depending on the characteristics of the agent, e.g., an AAV vector, an AAV capsid, or a composition, how the agent is administered, the degree of risk of disease, and the history, age, weight, family history, genetic makeup, the types of preceding or concomitant treatments, if any, and other individual characteristics of the patient to be treated.

The term “cellular reprogramming,” as used herein, refers to the process of altering a cell using reprogramming factors (e.g. reversing or preventing changes in cells that are causes of dysfunction, deterioration, cell death, senescence or aging). Cellular reprogramming may be complete reprogramming, such that a differentiated cell (e.g., somatic cell) is reprogrammed to a pluripotent stem cell. Cellular reprogramming may be incomplete, such that a differentiated cell (e.g., somatic cell) retains its cellular identity. Cellular reprogramming may be partial, e.g., a stem cell is not created, such that a cell is rejuvenated, or takes on more youthful attributes (e.g., increased survival, reduced inflammation). Cellular reprogramming may provide additional cellular functions, or prevent cellular senescence.

The term “rejuvenating a cell,” as used herein, is meant to include preventing or reversing the cellular causes of aging without inducing a pluripotent state.

A “pluripotent state” as used herein is meant to include a state in which the cell expresses at least one stem cell marker such as, but not limited to, Esrrb, Nanog, Lin28, TRA-1-60/TRA-1-81/TRA-2-54, SSEA1, or SSEA4. Methods of measuring the expression of stem cell markers on the cell are known in the art and include the methods described herein.

### II. Polynucleotides

Provided are polynucleotides for expression of cell rejuvenation promoting proteins in senescent cells. In some aspects, the polynucleotides comprise a promoter that is active in senescent cells. In some aspects, the promoter is mIR146A. In some aspects, the promoter is a promoter of a cyclin-dependent kinase inhibitor 2A (CDKN2A)/p16 gene. In some aspects, the promoter is mir146A. In some aspects, the promoter comprises a polynucleotide sequence that is at least 85%, 86%, 87%, 88%, 89%, 90%, 91%, 92%, 93%, 94%, 95%, 96%, 97%, 98%, or 99%, identical the human p16 promoter of SEQ ID NO: 1. In some aspects, the promoter comprises the polynucleotide sequence of SEQ ID NO: 1. In some aspects, the promoter comprises a polynucleotide sequence that is at least 85%, 86%, 87%, 88%, 89%, 90%, 91%, 92%, 93%, 94%, 95%, 96%, 97%, 98%, or 99%, identical the mouse p16 promoter of SEQ ID NO: 2. In some aspects, the promoter comprises the polynucleotide sequence of SEQ ID NO: 2.

In some aspects, the polynucleotide comprises a nucleic acid sequence encoding an Oct4 protein. In some aspects, the Oct4 protein is a human Oct4 protein. In some aspects, the polynucleotide comprises a nucleic acid sequence that is at least 85%, 86%, 87%, 88%, 89%, 90%, 91%, 92%, 93%, 94%, 95%, 96%, 97%, 98%, or 99%, identical to the human Oct4 sequence of SEQ ID NO: 3. In some aspects, the promoter comprises the nucleic acid sequence of SEQ ID NO: 3.

In some aspects, the polynucleotide comprises a nucleic acid sequence encoding a Sox2 protein. In some aspects, the Sox2 protein is a human Sox2 protein. In some aspects, the polynucleotide comprises a nucleic acid sequence that is at least 85%, 86%, 87%, 88%, 89%, 90%, 91%, 92%, 93%, 94%, 95%, 96%, 97%, 98%, or 99%, identical to the human Sox2 sequence of SEQ ID NO: 4. In some aspects, the promoter comprises the nucleic acid sequence of SEQ ID NO: 4.

In some aspects, the polynucleotide comprises a nucleic acid sequence encoding a Klf4 protein. In some aspects, the Klf4 protein is a human Klf4 protein. In some aspects, the polynucleotide comprises a nucleic acid sequence that is at least 85%, 86%, 87%, 88%, 89%, 90%, 91%, 92%, 93%, 94%, 95%, 96%, 97%, 98%, or 99%, identical to the human Klf4 sequence of SEQ ID NO: 5. In some aspects, the promoter comprises the nucleic acid sequence of SEQ ID NO: 5.

In some aspects, a nucleic acid sequence encoding an Oct4 protein and/or a nucleic acid sequence encoding a Sox2 protein and/or a nucleic acid sequence encoding a Klf4 protein are operably linked to a promoter that is active in senescent cells. In some aspects, the promoter is the p16 promoter. In some aspects, the nucleic acid sequence encoding an Oct4 protein and/or the nucleic acid sequence encoding a Sox2 protein and/or the nucleic acid sequence encoding a Klf4 protein are operably linked to the p16 promoter of SEQ ID NO: 1.

In some aspects, the polynucleotide further comprises a proteolytic cleavable site. In some aspects, the proteolytic cleavable site is a self-processing cleavage site or a furin cleavage site. In some aspects, the self-processing cleavage site is a P2A, E2A, F2A, or T2A peptide. In some aspects, the furin cleavage site comprise the consensus sequence RXK(R)R of SEQ ID NO: 6. Thus, the polypeptide produced when the polynucleotide is transcribed in a cell can be cleaved by proteases in the cell to release the Oct4, Sox2, and Klf4 proteins.

In some aspects, the nucleic acid sequence encoding an Oct4, a Sox2 and a Klf4 protein are ordered in a 5′-3′ direction on the polynucleotide such that a desired amount of each of Oct4, Sox2, and Klf4 is produced in a cell transduced with the polynucleotide. The amount of Oc4, Sox2, and Klf4 protein required in a specific cell type to induce partial reprogramming may vary and a polynucleotide can be chosen that provides optimal ratio of Oct4, Sox2, and Klf4 protein levels for partial reprogramming of the respective cell type. In some aspects, the level of protein produced from a polynucleotide is highest the closest a protein encoding polynucleotide sequence is located to the promoter sequence. In some aspects, the Oct4, Sox2, and Klf4 are located in 5′ to 3′ order from the promoter sequence.

In some aspects, the polynucleotide comprises a p16 promoter, a nucleic acid sequence encoding an Oct4 protein, a proteolytic cleavable site, and a nucleic acid sequence encoding a Sox2 protein.

In some aspects, the polynucleotide comprises a p16 promoter, a nucleic acid sequence encoding an Oct4 protein, a proteolytic cleavable site, and a nucleic acid sequence encoding a Klf4 protein.

In some aspects, the polynucleotide comprises a p16 promoter, a nucleic acid sequence encoding a Sox2 protein, a proteolytic cleavable site, and a nucleic acid sequence encoding a Klf4 protein.

In some aspects, the polynucleotide comprises a p16 promoter, a nucleic acid sequence encoding an Oct4 protein, a first proteolytic cleavable site, a nucleic acid sequence encoding a Sox2 protein, a second proteolytic cleavable site, and a nucleic acid sequence encoding a Klf4 protein.

In some aspects, the polynucleotide comprises a p16 promoter, a nucleic acid sequence encoding an Oct4 protein, a first proteolytic cleavable site, a nucleic acid sequence encoding a Klf4 protein, a second proteolytic cleavable site, and a nucleic acid sequence encoding a Sox2 protein in 5′-3′ direction.

In some aspects, the polynucleotide comprises a p16 promoter, a nucleic acid sequence encoding a Sox2 protein, a first proteolytic cleavable site, a nucleic acid sequence encoding an Oct4 protein, a second proteolytic cleavable site, and a nucleic acid sequence encoding a Klf4 protein in 5′-3′ direction.

In some aspects, the polynucleotide comprises a p16 promoter, a nucleic acid sequence encoding a Sox2 protein, a first proteolytic cleavable site, a nucleic acid sequence encoding a Ktlf4 protein, a second proteolytic cleavable site, and a nucleic acid sequence encoding an Oct4 protein in 5′-3′ direction.

In some aspects, the polynucleotide comprises a p16 promoter, a nucleic acid sequence encoding a Klf4 protein, a first proteolytic cleavable site, a nucleic acid sequence encoding an Oct4 protein, a second proteolytic cleavable site, and a nucleic acid sequence encoding a Sox2 protein in 5′-3′ direction.

In some aspects, the polynucleotide comprises a p16 promoter, a nucleic acid sequence encoding a Klf4 protein, a first proteolytic cleavable site, a nucleic acid sequence encoding a Sox2 protein, a second proteolytic cleavable site, and a nucleic acid sequence encoding an Oct4 protein in 5′-3′ direction.

In some aspects, the p16 promoter is a human or murine p16 promoter.

In some aspects, the first proteolytic site is a P2A peptide, an E2A peptide, a F2A peptide, a T2A peptide or a RXK(R)R consensus sequence of SEQ ID NO: 6.

In some aspects, the second proteolytic site is a P2A peptide, an E2A peptide, a F2A peptide, a T2A peptide or a RXK(R)R consensus sequence of SEQ ID NO: 6.

In some aspects, the polynucleotide comprises a second p16 promoter.

In some aspects, the polynucleotide comprises a first p16 promoter, a nucleic acid sequence encoding an Oct4 protein, a second p16 promoter, a nucleic acid sequence encoding a Sox2 protein, a proteolytic cleavable site, and a nucleic acid sequence encoding a Klf4 protein in 5′-3′ direction.

In some aspects, the polynucleotide comprises a first p16 promoter, a nucleic acid sequence encoding an Oct4 protein, a second p16 promoter, a nucleic acid sequence encoding a Klf4 protein, a proteolytic cleavable site, and a nucleic acid sequence encoding a Sox2 protein in 5′-3′ direction.

In some aspects, the polynucleotide comprises a first p16 promoter, a nucleic acid sequence encoding an Oct4 protein, a proteolytic cleavable site, a nucleic acid sequence encoding a Sox2 protein, a second promoter, and a nucleic acid sequence encoding a Klf4 protein in 5′-3′ direction.

In some aspects, the polynucleotide comprises a first p16 promoter, a nucleic acid sequence encoding a Sox2 protein, a proteolytic cleavable site, a nucleic acid sequence encoding an Oct4 protein, a second promoter, and a nucleic acid sequence encoding a Klf4 protein in 5′-3′ direction.

In some aspects, the polynucleotide comprises a first p16 promoter, a nucleic acid sequence encoding an Oct4 protein, a proteolytic cleavable site, a nucleic acid sequence encoding a Klf4 protein, a second promoter, and a nucleic acid sequence encoding a Sox2 protein in 5′-3′ direction.

In some aspects, the polynucleotide comprises a first p16 promoter, a nucleic acid sequence encoding a Klf4 protein, a proteolytic cleavable site, a nucleic acid sequence encoding an Oct4 protein, a second promoter, and a nucleic acid sequence encoding a Sox2 protein in 5′-3′ direction.

In some aspects, the first and the second promoter initiate transcription in the same direction.

In some aspects, the first and the second promoter initiate transcription in different directions.

In some aspects, the polynucleotide further comprises a polyadenylation signal sequence. In some aspects, the polyadenylation signal sequence is an SV40 polyadenylation signal sequence, a human growth hormone polyadenylation signal sequence, or a bovine growth hormone polyadenylation signal sequence.

In some aspects, the polynucleotide further comprises a WPRE sequence.

In some aspects, the polynucleotide further comprises an IRES.

In some aspects, the polynucleotide comprises a p16 promoter, a nucleic acid sequence encoding an Oct4 protein, a first IRES, a nucleic acid sequence encoding a Sox2 protein, a second IRES, and a nucleic acid sequence encoding a Klf4 protein in 5′-3′ direction.

In some aspects, the polynucleotide comprises a p16 promoter, a nucleic acid sequence encoding an Oct4 protein, a first IRES, a nucleic acid sequence encoding a Klf4 protein, a second IRES, and a nucleic acid sequence encoding a Sox2 protein in 5′-3′ direction.

In some aspects, the polynucleotide comprises a p16 promoter, a nucleic acid sequence encoding a Sox2 protein, a first IRES, a nucleic acid sequence encoding an Oct4 protein, a second IRES, and a nucleic acid sequence encoding a Klf4 protein in 5′-3′ direction.

In some aspects, the polynucleotide comprises a p16 promoter, a nucleic acid sequence encoding a Sox2 protein, a first IRES, a nucleic acid sequence encoding a Klf4 protein, a second IRES, and a nucleic acid sequence encoding an Oct4 protein in 5′-3′ direction.

In some aspects, the polynucleotide comprises a p16 promoter, a nucleic acid sequence encoding a Klf4 protein, a first IRES, a nucleic acid sequence encoding an Oct4 protein, a second IRES, and a nucleic acid sequence encoding a Sox2 protein in 5′-3′ direction.

In some aspects, the polynucleotide comprises a p16 promoter, a nucleic acid sequence encoding a Klf4 protein, a first IRES, a nucleic acid sequence encoding a Sox2 protein, a second IRES, and a nucleic acid sequence encoding an Oct4 protein in 5′-3′ direction.

In some aspects, the polynucleotide comprises a first p16 promoter, a nucleic acid sequence encoding an Oct4 protein, a second p16 promoter, a nucleic acid sequence encoding a Sox2 protein, an IRES, and a nucleic acid sequence encoding a Klf4 protein in 5′-3′ direction.

In some aspects, the polynucleotide comprises a first p16 promoter, a nucleic acid sequence encoding an Oct4 protein, a second p16 promoter, a nucleic acid sequence encoding a Klf4 protein, an IRES, and a nucleic acid sequence encoding a Sox2 protein in 5′-3′ direction.

In some aspects, the polynucleotide comprises a first p16 promoter, a nucleic acid sequence encoding an Oct4 protein, an IRES, a nucleic acid sequence encoding a Sox2 protein, a second promoter, and a nucleic acid sequence encoding a Klf4 protein in 5′-3′ direction.

In some aspects, the polynucleotide comprises a first p16 promoter, a nucleic acid sequence encoding a Sox2 protein, an IRES, a nucleic acid sequence encoding an Oct4 protein, a second promoter, and a nucleic acid sequence encoding a Klf4 protein in 5′-3′ direction.

In some aspects, the polynucleotide comprises a first p16 promoter, a nucleic acid sequence encoding an Oct4 protein, an IRES, a nucleic acid sequence encoding a Klf4 protein, a second promoter, and a nucleic acid sequence encoding a Sox2 protein in 5′-3′ direction.

In some aspects, the polynucleotide comprises a first p16 promoter, a nucleic acid sequence encoding a Klf4 protein, an IRES, a nucleic acid sequence encoding an Oct4 protein, a second promoter, and a nucleic acid sequence encoding a Sox2 protein in 5′-3′ direction.

In some aspects, the polynucleotide further comprises a nucleic acid sequence encoding a marker protein. In some aspects, the marker protein is a β-galactosidase protein, a green fluorescent protein, a red fluorescent protein, a yellow fluorescent protein, cyan fluorescent protein, or a blue fluorescent protein, a tdTomato protein, or a mCherry protein.

In some aspects, the polynucleotide comprises an intron sequence. In some aspects, the intron sequence comprises a CMV intronic sequence, a β-actin intronic sequence, a chicken β-actin intron, a SV40 enhancer sequence, or a combination thereof.

### II. Vectors and Cells

Also provided are vectors comprising a polynucleotide as described herein.

In some aspects, the vectors are viral vectors. In some aspects, the vectors are non-viral vectors. In some aspects, the vectors are lipids. In some aspects, the vectors polymers.

In some aspects, the viral vectors are vector is adeno-associated viral (AAV) vectors, adenoviral vectors, lentiviral vectors, or retroviral vectors. In some aspects, the AAV vector is a AAV1, AAV2, AAV3, AAV4, AAV5, AAV6, AAV7, AAV8, AAVRH8, AAVrh9, AAV9, AAVrh10, AAV10, AAVRH10, AAV11, AAV12, or AAV-DJ vector.

In some aspects, an AAV vector with broad target spectrum is chosen to transduce various cell types. In some aspects, the AAV vector is an AAV-DJ vector.

In some aspects, an AAV vector with a specific target spectrum is chosen to transduce select cell types. The target specificity of different AAV vectors is known in the art (see, e.g. AAV Production Protocol, Genemedi Biotech, Inc. 2018.).

In some aspects, the AAV vector is modified to target a select cell type or select cell types. For example, an AAV cap sequence can be modified to remove a cell-targeting epitope from the capsid and introduce an alternative cell-targeting sequence in the capsid. AAV cap sequences modified in this manner are known in the art.

In some aspects, the non-viral vector is a plasmid DNA, a RNA a cationic polymer, a lipid, a lipopolymer or a chemical derivative thereof.

In some aspects, the cationic polymer is present in an amount sufficient to produce a ratio of amine nitrogen in the cationic polymer to phosphate in the DNA plasmid vector or RNA vector from about 0.1:1 to about 100:1.

In some aspects, the cationic polymer is present in an amount sufficient to produce a ratio of amine nitrogen in the cationic polymer to phosphate in the DNA plasmid vector or RNA vector from about 0.1:1 to about 10:1.

In some aspects, the cationic polymer is present in an amount sufficient to produce a ratio of amine nitrogen in the cationic polymer to phosphate in the DNA plasmid vector or RNA vector from about 0.1:1 to about 5:1.

In some aspects, the non-viral vector comprises about 0.5 mg/ml to about 5.0 mg/ml polynucleotide complexed with the cationic polymer. In some aspects, the cationic polymer is a poly(ethylenimine) (PEI) polymer, poly-L-lysine, polyamidoamine, diethylaminoethyl dextrans, chitosan, poly(dimethyl-aminoethyl methacrylates) or derivatives thereof.

Further provided are cells comprising a polynucleotide as described herein or a vector as described herein. In some aspects, the cells can be bacterial cells, yeast cells, fungal cells, insect cells, or mammalian cells.

In some aspects, the cells comprising a polynucleotide as described herein can be used to manufacture a viral vector. In some aspects, the cells are insect cells comprising a polynucleotide as described herein and further viral vector production components and are used to prepare a viral vector as described herein. In some aspects, the cells are mammalian cells comprising a polynucleotide as described herein and further viral vector production components and are used to prepare a viral vector as described herein.

In some aspects, the cells are insect cells comprising a polynucleotide as described herein and AAV viral vector production components and are used to prepare AAV vectors.

In some aspects, the cells are mammalian cells comprising a polynucleotide as described herein and AAV viral vector production components and are used to prepare AAV vectors.

In some aspects, the cells comprising a polynucleotide as described herein can be used to treat a subject. In some aspects, the cells comprising a polynucleotide as described herein can be administered to a subject in need of reversing senescence, wherein the cells after being administered express the proteins encoded by the polynucleotides described herein and the expressed proteins reverse cellular processes associated with a senescent phenotype. In some aspects, the cells comprising a polynucleotide as described herein after being administered to a subject replicate. In some aspects, the cells comprising a polynucleotide as described herein can be hematopoietic progenitor cells. In some aspects, the cells comprising a polynucleotide as described herein can be hematopoietic stem cells. In some aspects, the cells comprising a polynucleotide as described herein can be mesenchymal/stromal stem cells. In some aspects, the cells comprising a polynucleotide as described herein can be adipose stem cells.

### IV. Compostions and Kits

Also provided are compositions comprising polynucleotides as described herein and/or vectors as described herein. In some aspects, the composition are gene therapy compositions. In some aspects, the compositions comprise a polynucleotide as described herein and a delivery agent or the vector as described herein.

In some aspects, the delivery agent is a cationic polymer. In some aspects, the delivery agent is a lipid, a lipopolymer or a chemical derivative thereof.

In some aspects, the cationic polymer of the composition is present in an amount sufficient to produce a ratio of amine nitrogen in the cationic polymer to phosphate in the DNA plasmid or RNA from about 0.1:1 to about 100:1.

In some aspects, the cationic polymer of the composition is present in an amount sufficient to produce a ratio of amine nitrogen in the cationic polymer to phosphate in the DNA plasmid or RNA from about 0.1:1 to about 10:1.

In some aspects, the cationic polymer of the composition is present in an amount sufficient to produce a ratio of amine nitrogen in the cationic polymer to phosphate in the DNA plasmid or RNA from about 0.1:1 to about 5:1.

In some aspects, the polynucleotide as described herein is present at about 0.5 mg/ml to about 5.0 mg/ml complexed with the cationic polymer of the composition. In some aspects, the cationic polymer of the composition is a poly(ethylenimine) (PEI) polymer, poly-L-lysine, polyamidoamine, diethylaminoethyl dextrans, chitosan, poly(dimethyl-aminoethyl methacrylates) or a derivative thereof.

Further provided are pharmaceutical compositions. In some aspects, the pharmaceutical compositions comprise a pharmaceutically acceptable carrier or excipient.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the polynucleotides of the instant disclosure include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

In some aspects, the compositions further comprise wetting agents, emulsifiers and lubricants, such as sodium lauryl sulfate and magnesium stearate, as well as coloring agents, release agents, coating agents, sweetening, flavoring and perfuming agents, preservatives and antioxidants.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In some aspects, the compositions of the disclosure include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In some aspects, a composition of the disclosure comprises an excipient selected from cyclodextrins, celluloses, liposomes, micelle forming agents, e.g., bile acids, and polymeric carriers, e.g., polyesters and polyanhydrides; and a polynucleotide described herein. In some aspects, a composition renders orally bioavailable a polynucleotide described herein.

Methods of preparing these compositions or pharmaceutical compositions include a step of bringing into association a polynucleotide of the present disclosure with the carrier and, optionally, one or more accessory ingredients. In general, the compositions are prepared by uniformly and intimately bringing into association a polynucleotide of the present disclosure with liquid carriers, or finely divided solid carriers, or both, and then, if necessary, shaping the product.

Compositions of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A polynucleotide as described herein may also be administered as a bolus, electuary or paste.

In solid dosage forms of the disclosure for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof, (10) coloring agents; and (11) controlled release agents such as crospovidone or ethyl cellulose. In the case of capsules, tablets and pills, the pharmaceutical compositions may also comprise buffering agents. Solid pharmaceutical compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid pharmaceutical compositions which can be dissolved in sterile water, or some other sterile injectable medium immediately before use. These pharmaceutical compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral pharmaceutical compositions can also include adjuvants such as wetting agents, emulsifying and suspending agents, sweetening, flavoring, coloring, perfuming and preservative agents.

Suspensions, in addition to the polynucleotide, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more polynucleotides described herein with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of a polynucleotide or vector as described herein include powders, sprays, ointments, pastes, creams, lotions, gels, solutions, patches and inhalants. The polynucleotides may be mixed under sterile conditions with a pharmaceutically-acceptable carrier, and with any preservatives, buffers, or propellants which may be required. The ointments, pastes, creams and gels may contain, in addition to a polynucleotide described herein, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a polynucleotide described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a polynucleotide or vector described herein to the body. Such dosage forms can be made by dissolving or dispersing the polynucleotide or vector in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more polynucleotides or vectors as described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the compositions isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These pharmaceutical compositions may also contain adjuvants such as preservatives, wetting agents, emulsifying agents and dispersing agents. Prevention of the action of microorganisms upon the polynucleotides or vectors described herein may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some aspects, in order to prolong the effect of a polynucleotide or vector described herein, or to slow the absorption from subcutaneous or intramuscular injection, a liquid suspension of crystalline or amorphous material having poor water solubility may be used among other methods known in the art. The rate of absorption of a composition comprising a polynucleotide or vector as described herein then depends upon its rate of dissolution which, in turn, may depend upon crystal size and crystalline form. Alternatively, delayed absorption of a parenterally-administered drug form can be accomplished by dissolving or suspending the polynucleotide or vector as described herein in an oil vehicle.

Injectable depot forms may be made by forming microencapsule matrices of the polynucleotides in biodegradable polymers such as polylactide-polyglycolide. Depending on the ratio of polynucleotide or vector to polymer, and the nature of the particular polymer employed, the rate of polynucleotide or vector release can be controlled. Examples of other biodegradable polymers include poly(orthoesters) and poly(anhydrides). Depot injectable formulations may also prepared by entrapping the polynucleotide or vector in liposomes or microemulsions that are compatible with body tissues.

When the polynucleotides or vectors described herein are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

As noted above, the formulations or preparations of the present disclosure may be given orally, parenterally, topically, or rectally. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

Regardless of the route of administration selected, the polynucleotides or vectors described herein, which may be used in a suitable hydrated form, and/or the pharmaceutical compositions of the present disclosure, may be formulated into pharmaceutically-acceptable dosage forms by conventional methods known to those of skill in the art. Actual dosage levels of the active ingredients in the pharmaceutical compositions of this disclosure may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular polynucleotide or vector employed, or the ester, salt or amide thereof, the route of administration, the time of administration, the rate of excretion or metabolism of the particular polynucleotide or vector being employed, the rate and extent of absorption, the duration of the treatment, other drugs, compounds and/or materials used in combination with the particular polynucleotide or vector employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compositions of the disclosure employed in the pharmaceutical composition at levels lower than that required in order to achieve the desired therapeutic effect and gradually increase the dosage until the desired effect is achieved. In general, a suitable daily dose of a composition of the disclosure will be that amount of the polynucleotide or vector which is the lowest dose effective to produce a therapeutic effect. Such an effective dose will generally depend upon the factors described above. Generally, oral and parenteral doses of the composition of this disclosure for a patient, when used for the indicated effects, will range from about 0.0001 to about 100 mg per kilogram of body weight per day.

Polynucleotides can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In some aspects, microemulsification technology may be utilized to improve bioavailability. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other benefits, microemulsification provides enhanced bioavailability by preferentially directing absorption to the lymphatic system instead of the circulatory system, which thereby bypasses the liver, and prevents degradation of, e.g., viral vector particles in the hepatobiliary circulation.

In some aspects, the formulations contain micelles formed from a polynucleotide or vector described herein and at least one amphiphilic carrier, in which the micelles have an average diameter of less than about 100 nm. In some aspects, micelles having an average diameter less than about 50 nm are used with polynucleotides or vectors described herein. In some aspects, micelles having an average diameter less than about 30 nm, or even less than about 20 nm are used.

While all suitable amphiphilic carriers are contemplated, the presently preferred carriers are generally those that have Generally-Recognized-as-Safe (GRAS) status, and that can both solubilize the polynucleotides or vectors described herein and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

In some aspects, the delivery may occur by use of liposomes, nanocapsules, microparticles, microspheres, lipid particles, vesicles, and the like, for the introduction of the pharmaceutical compositions into suitable host cells. In particular, the pharmaceutical compositions may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use with polynucleotides or vectors described herein are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In some aspects, polymers have a molecular weight of from about 100 or 120 daltons up to about 5,000 or 10,000 daltons, or from about 300 daltons to about 5,000 daltons. In some aspects, the polymer is polyethyleneglycol having a molecular weight of from about 100 to about 5,000 daltons, or having a molecular weight of from about 300 to about 5,000 daltons. In some aspects, the polymer is polyethyleneglycol of 750 daltons (PEG(750)). Polymers may also be defined by the number of monomers therein; in some aspects, polymers of at least about three monomers, such PEG polymers consisting of three monomers (approximately 150 daltons) are used.

Other hydrophilic polymers which may be suitable for use in the present disclosure include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In some aspects, a composition of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In some aspects, a composition of the present disclosure comprises a cationic polymer. In some aspects, the cationic polymer comprises a polyethyleneimine (PEI) backbone. In some aspects, the PEI backbone is linked to a lipid or polyethylene glycol. In some aspects, the cationic polymer comprises a cationic dextran, a cationic chitosan, a cationic gelatin, a cationic cellulose, or a cationic cyclodextrin.

In some aspects, a composition of the present disclosure comprises a cyclodextrin. Cyclodextrins are cyclic oligosaccharides allow complexation with a variety of relatively hydrophobic compounds by Van der Waals interactions and by hydrogen bond formation. For a general review of the chemistry of cyclodextrins, see, Wenz, Agnew. Chem. Int. Ed. Engl., 33:803-822 (1994). The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility. Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

In some aspects, a composition of the present disclosure comprises a liposome. Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

In some aspects, compositions comprising liposomes containing a polynucleotide or vector as described herein are used where the liposome membrane is formulated to provide a liposome with increased carrying capacity. Alternatively or in addition, the composition of the present disclosure may be contained within, or adsorbed onto, the liposome bilayer of the liposome. A polynucleotide described herein may be aggregated with a lipid surfactant and carried within the liposome's internal space; in these cases, the liposome membrane is formulated to resist the disruptive effects of the active agent-surfactant aggregate.

In some aspects, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents, such as polynucleotides or vectors, contained within liposomes described herein are in solubilized form. Aggregates of surfactant and active agent (such as emulsions or micelles containing the polynucleotide or vector) may be entrapped within the interior space of liposomes according to the present disclosure. A surfactant acts to disperse and solubilize the polynucleotide or vector, and may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present disclosure.

Liposomes according to the present disclosure may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993. For example, liposomes described herein may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In some aspects of the formulation procedure, the active agent is first dispersed by sonication in a lysophosphatidylcholine or other low CMC surfactant (including polymer grafted lipids). The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In some aspects, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In some aspects, reagents such as DharmaFECT® and Lipofectamine® may be utilized to introduce polynucleotides or proteins into cells.

The release characteristics of a formulation of the present disclosure depend on the encapsulating material, the concentration of encapsulated polynucleotides, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of a composition by diffusion from the capsule. Excipients which modify the solubility of the composition can also be used to control the release rate. Agents which enhance degradation of the matrix or release from the matrix can also be incorporated. They can be added to the composition, added as a separate phase (i.e., as particulates), or can be co-dissolved in the polymer phase depending on the composition. In some aspects, the amount is between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

In some aspects, uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

In some aspects, a polynucleotide or vector may be formulated to be contained within, or, adapted to release by a surgical or medical device or implant. In some aspects, an implant may be coated or otherwise treated with a polynucleotide or vector. For example, hydrogels, or other polymers, such as biocompatible and/or biodegradable polymers, may be used to coat an implant with the pharmaceutical compositions of the present disclosure (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In some aspects, the polynucleotides for use according to the disclosure may be formulated for administration in any convenient way for use in human or veterinary medicine, by analogy with other pharmaceuticals. The polynucleotides or vectors and their corresponding formulations may be administered alone or in combination with other therapeutic strategies in the treatment of progeria syndrome, premature aging, signs and/or symptoms of aging such as stem cell therapies, administration of reverse transcriptase inhibitors, and therapies that counter the aging process in cells.

### V. Methods of Making Recombinant AAV particles

Provided are recombinant viral particles for administration to a subject in need of cellular reprogramming from a senescent stage phenotype to a non-senescent stage phenotype. In some aspects, the recombinant viral particles are AAV particles.

In some aspects, a method for making a rAAV comprises (i) providing a cell with adeno-associated virus (AAV) rep genes, AAV cap genes, a nucleic acid comprising the polynucleotide as described herein, the vector as described herein, and at least one AAV serotype 2 inverted terminal repeat, (ii) providing helper functions for generating a productive AAV infection; (ii) allowing assembly of the AAV; and (iii) collecting the rAAV.

In some aspects, the AAV rep genes and AAV cap genes are provided by a plasmid.

In some aspects, the AAV rep genes and AAV cap genes are provided by an adenovirus vector.

In some aspects, the AAV rep genes and AAV cap genes are stably integrated into the genome of the cell.

In some aspects, the helper functions are provided by a plasmid.

In some aspects, the helper functions are provided by an adenovirus vector.

In some aspects, provided are rAAV produced by the method described herein.

### VI. Methods of Treatment

Provided are methods of treating, preventing, or inhibiting premature aging or an age-related disease in a subject, the method comprising administering a therapeutically effective amount of a polynucleotide described herein, a vector described herein, a cell described herein, a composition described herein, a rAAV described herein, a pharmaceutical composition described herein to the subject in an amount effective to treat, prevent, or inhibit premature aging or an age-related disease in the subject.

In some aspects, the subject has a progeroid syndrome.

In some aspects, the progeroid syndrome is selected from the group consisting of Hutchinson-Gilford progeria syndrome; Werner syndrome; atypical progeria, mandibuloacral dysplasia type A; mandibuloacral dysplasia type B; mandibuloacral dysplasia associated to MTX2; mandibular hypoplasia, progeroid features and lipodystrophy syndrome (MDPL); Nestor-Guillermo progeria syndrome; and restrictive dermopathy.

In some aspects, the subject has a mutation in a LMNA gene, ZMPSTE24 gene, BANF1 gene, POLD1 gene, MTX2 gene, or WRN gene.

In some aspects, the subject has signs and/or symptoms of aging.

In some aspects, methods of treating, preventing or inhibiting signs or symptoms of aging in a subject are provided, the methods comprising administering a therapeutically effective amount of a polynucleotide described herein, a vector described herein, a cell described herein, a composition described herein, a rAAV described herein, a pharmaceutical composition described herein to the subject in an amount effective to treat, prevent, or inhibit signs and symptoms of aging in the subject.

In some aspects, a method for treating cellular senescence-related aging in a subject in need thereof is provided. In some aspects, the method comprises administering to subject a therapeutically effective amount of the polynucleotide as described herein, the vector as described herein, the cell as described herein, the composition as described herein, the rAAV as described herein, or the pharmaceutical composition as described herein.

In some aspects, a method of reprogramming a senescent cell to a non-senescent stage phenotype is provided. In some aspects, provided is a method of reprogramming a p16-expressing cell to a non-p16-expressing cell. In some aspects, the p16-expressing cell is an aged cell. In some aspects, the p16-expressing cell is a naturally aged cell. In some aspects, the p16-expressing cell is a prematurely aged cell. In some aspects, provided is a method of reprogramming a p16-expressing cell in an aged organism. In some aspects, the method partially or fully reverses aging in the cell. In some aspects, the method partially or fully reverses aging in a naturally aged cell. In some aspects, the method partially or fully reverses aging in a prematurely aged cell. In some aspects, the method partially or fully reverses aging in a cell of an aged organism. In some aspects, the method comprises contacting a senescent cell and/or aged cell and/or aging cell with the polynucleotide as described herein, the vector as described herein, the cell as described herein, the composition as described herein, or the rAAV as described herein, or the pharmaceutical composition as described herein in an amount effective to partially reprogram the senescent cell to a non-senescent stage phenotype, and/or the aged cell to a non-aged or less-aged phenotype, and/or the aging cell to a non-aging phenotype.

In some aspects, a method for reprogramming senescent cells in a subject to a non-senescent stage phenotype is provided. In some aspects, the method comprises administering to the subject in need thereof a therapeutically effective amount of the polynucleotide as described herein, the vector as described herein, the cell as described herein, the composition as described herein, the rAAV as described herein, or the pharmaceutical composition as described herein, and partially reprogramming senescent cells of the subject to a non-senescent stage phenotype.

In some aspects, a method for inhibiting Senescence-Associated Secretory Phenotype (SASP) activity in a cell is provided. In some aspects, the method comprises contacting the cell with the polynucleotide as described herein, the vector as described herein, the cell as described herein, the composition as described herein, the rAAV as described herein, or the pharmaceutical composition as described herein, in an amount effective to inhibit SASP activity in the cell.

In some aspects, a method for rejuvenating a senescent cell to a non-senescent stage phenotype is provided. In some aspects, the method comprises contacting a senescent cell with the polynucleotide as described herein, the vector as described herein, the cell as described herein, the composition as described herein, the rAAV as described herein, or the pharmaceutical composition as described herein in an amount effective to partially reprogram the senescent cell. In some aspects, the method further comprises contacting the senescent cell with the polynucleotide as described herein, or the vector as described herein in an amount effective to inhibit SASP activity in the senescent cell.

In some aspects, a method for rejuvenating senescent cells in a subject to a non-senescent stage phenotype is provided. In some aspects, the method comprises administering to a subject in need thereof a therapeutically effective amount of the polynucleotide as described herein, the vector as described herein, the cell as described herein, the composition as described herein, the rAAV as described herein, or the pharmaceutical composition as described herein, to partially reprogram the senescent cells; and administering to the subject a therapeutically effective amount of the polynucleotide as described herein, the vector as described herein, the cell as described herein, the composition as described herein, the rAAV as described herein, or the pharmaceutical composition as described herein, to inhibit SASP activity.

In some aspects, a method for rejuvenating aged cells in a subject to a non-aged or less aged phenotype is provided. In some aspects, the method comprises administering to a subject in need thereof a therapeutically effective amount of the polynucleotide as described herein, the vector as described herein, the cell as described herein, the composition as described herein, the rAAV as described herein, or the pharmaceutical composition as described herein, to partially reprogram the aged cells. In some aspects, the aged cell is a naturally aged cell, a prematurely aged cell, and/or a cell in an aged organism. In some aspects, the aged cell is a p16-expressing cell. In some aspects, administration of the composition as described herein, the rAAV as described herein, or the pharmaceutical composition as described herein to an aged cell partially or fully reverts the aged cell to a non-aged or less aged cell. In some aspects, administration of the composition as described herein, the rAAV as described herein, or the pharmaceutical composition as described herein to a p16-expressing cell partially or fully reverts the p16-expressing cell to a non-p16-expressing cell. In some aspects, administration of the composition as described herein, the rAAV as described herein, or the pharmaceutical composition as described herein to a p16-expressing cell reduces p16 expression in the cell.

In some aspects, the administration induces increased epidermal thickness, reduced mesangial area in the kidney, increased structural arrangement of hepatocytes in the liver, rescued lymphoid depletion in germinal centers of the spleen, reduced level of inflammatory markers, increased number of hematopoietic stem cells, restoration of bone marrow tissue and/or an increased activation of DNA repair pathways in the subject.

In some aspects, the reduced level of inflammatory markers comprises reduced neutrophil invasion in the liver and a reduced inflammatory signature in the spleen.

In some aspects, a method for increasing age-related survival of a subject is provided. In some aspects, the method comprises administering to a subject in need thereof a therapeutically effective amount of the polynucleotide as described herein, the as described herein, the cell as described herein, the composition as described herein, the rAAV as described herein, or the pharmaceutical composition as described herein, and reprogramming senescent cells in the subject, whereby the age-related survival of the subject is increased.

In some aspects, the subject is a human.

In some aspects, provided are methods of treatment comprising contacting a cell with a polynucleotide as described herein, a vector as described herein, a cell as described herein, a composition as described herein, a rAAV as described herein, or a pharmaceutical composition as described herein in an amount effective to partially reprogram the cell. In some aspects, the contacting takes place ex vivo, e.g., in a cell culture dish. In some aspects, the contacted cell is subsequently administered to a subject in need of a partially reprogrammed cell.

In some aspects, a method of treatment is provided, the method comprising contacting a cell with a polynucleotide as described herein, a vector as described herein, a cell as described herein, a composition as described herein, a rAAV as described herein, or a pharmaceutical composition as described herein in an amount effective to partially reprogram the cell. In further aspects, the method comprises and administering the cell to a subject in need of a partially reprogrammed cell. In some aspects, the cell is a fibroblast. In some aspects, the fibroblast is a mammalian fibroblast. In some aspects, the fibroblast is a human fibroblast.

In some aspects, provided is a use of a polynucleotide as described herein, a vector as described herein, a cell as described herein, a composition as described herein, a rAAV as described herein, or a pharmaceutical composition as described herein for treating cellular senescence-related aging.

In some aspects, provided is a use of a polynucleotide as described herein, a vector as described herein, a cell as described herein, a composition as described herein, a rAAV as described herein, or a pharmaceutical composition as described herein in the manufacture of a medicament for treating cellular senescence-related aging.

In some aspects, provided is an article of manufacture comprising a polynucleotide as described herein, a vector as described herein, a cell as described herein, a composition as described herein, a rAAV as described herein, or a pharmaceutical composition as described herein.

In some aspects, the methods further comprise administering an additional therapeutic agent to the subject.

In some aspects, a polynucleotide described herein, a vector described herein, a cell described herein, a composition described herein, a rAAV described herein, a pharmaceutical composition described herein are administered by an intravenous, intramuscular, intradermal, subcutaneous, intraperitoneal, or intranasal route.

In some aspects, the process of administration can be varied, depending on the composition, or compositions, and the desired effect. Thus, the process of administration involves administering a polynucleotide described herein, a vector described herein, a cell described herein, a composition described herein, a rAAV described herein, a pharmaceutical composition described herein to a patient in need of such treatment.

In some aspects, administration can be accomplished by any means appropriate for the therapeutic agent, for example, by parenteral, mucosal, pulmonary, subcutaneous, intradermal, topical, catheter-based, or oral means of delivery. Parenteral delivery can include for example, subcutaneous, intravenous, intramuscular, intra-arterial, intraperitoneal, intralymphatic, and injection into the tissue of an organ. Mucosal delivery can include, for example, intranasal delivery, administration into an airway of a patient, i.e., nose, sinus, throat, lung, for example, as nose drops, by nebulization, vaporization, or other methods known in the art. Oral or intranasal delivery can include the administration of a propellant. Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid, according to methods and compositions described herein.

In some aspects, a therapeutically effective amount of a polynucleotide described herein, a vector described herein, a cell described herein, a composition described herein, a rAAV described herein, a pharmaceutical composition described herein is administered by an intravenous, intramuscular, intradermal, subcutaneous, intraperitoneal, or intranasal route.

In some aspects, a polynucleotide described herein, a vector described herein, a cell described herein, a composition described herein, a rAAV described herein, a pharmaceutical composition described herein is delivered to a subject or a tissue of a subject as a combination therapy.

In some aspects, a polynucleotide described herein, a vector described herein, a cell described herein, a composition described herein, a rAAV described herein, a pharmaceutical composition described herein is delivered to a subject or a tissue of a subject together with at least one additional therapeutic agent. In some aspects, a polynucleotide described herein, a vector described herein, a cell described herein, a composition described herein, a rAAV described herein, a pharmaceutical composition described herein is administered to a subject before, after, or together with at least one additional therapeutic agent.

In some aspects a polynucleotide described herein, a vector described herein, a cell described herein, a composition described herein, a rAAV described herein, a pharmaceutical composition described herein and the at least one additional therapeutic agent can be administered by the same route or by different routes; at essentially the same time (i.e. simultaneously, concurrently) or at different times (e.g. sequentially, successively, alternately, consecutively, or according to any other sort of alternating regime). In some aspects, the additional therapeutic may be administered prior, concurrently, or subsequently to the administration of the polynucleotides or vectors described herein. For example, in some aspects, the polynucleotides or vectors are administered to a patient that is on a background anti-aging therapy. For example, in some aspects, the patient has been treated with an antiaging therapy prior to administration of a polynucleotide or vector as described herein and continues to receive the antiaging therapy.

In some aspects, a first therapeutic composition of the combination can be administered by intravenous injection while the additional therapeutic composition of the combination can be administered intradermally. Alternatively, for example, all therapeutic compositions can be administered by intravenous injection or all therapeutic compositions can be administered by intradermal injection.

The routes of administration described are intended only as a guide since a skilled practitioner will be able to determine readily the optimum route of administration and any dosage for any particular animal and condition.

### EXAMPLES

### Materials and Methods

### Cell Culture

Human primary lung fibroblast IMR90 cells were obtained from ATCC and were cultured in DMEM (Gibco), 10% FBS(Gibco), 1% penicillin/streptomycin (Invitrogen), along with 1× Glutamax (Invitrogen) and Non-Essential amino acids (Invitrogen). HEK293A cells were used for AAV production and maintained in the medium containing DMEM, 10% FBS; 1% penicillin/streptomycin. All cells were cultured at 37 °C with 7% C02 in a humid incubator. Cells were split in regular intervals when they reaching around 70% confluency and the medium was changed every 2 days.

### AAV Production

AAVs were prepared using HEK293-AAV cells (Cell Biolabs, Inc.) as described with minor modifications (Grieger, J. C., Choi, V. W. & Samulski, R. J. Nat Protoc 1, 1412-1428, 2006). PEI (MW 40000) was used to transfect cells followed by CsCl gradient purification. Virus titer was determined via qPCR using primers; ITR-F:5′ GGAACCCCTAGTGATGGAGTT 3′ and ITR-R: 5′ CGGCCTCAGTGAGCGA 3′.

### Inhibitors

Topoisomerase inhibitor Etoposide (Sigma-Aldrich, St. Louis, USA) was dissolved in sterile PBS and diluted to a working concentration of 15 μM before use.

### Plasmid Vectors

All plasmids used in this study are shown in FIG. 12-17.

### Quantitative Real Time PCR

mRNA levels were quantified as previously described (Sahu, S. K. et al. Nat Commun 8, 1523, 2017). In brief, total RNA was prepared using Trizol (Invitrogen) and was reverse transcribed with a First Strand cDNA Synthesis Kit (Fermentas). The transcripts were quantified via PCR using SYBR green PCR MasterMix (ABI) on a ViiA7 PCR machine (Life Technologies). Human or mouse GAPDH and TBP primers were used for normalization. The sequence details of primers used in this study are listed below. Mouse real-Time PCT primers are listed in Table 1.

TABLE 1

| | |
|---|---|
| Cdkn2a FP | GTGTGCATGACGTGCGGG |
| Cdkn2a RP | GTTGCCCATCATCATCACCTGAA |
| Ctcf FP | GCCTGCAGCCACTGCGACAA |
| Ctcf RP | CCATTGTGTTCCGGCGGGTGA |
| Il1a FP | AGGAGAAGACCAGCCCGTGT |
| Il1a RP | TGCCAGGTGCACCCGACTTT |
| Il1b FP | AAAGACGGCACACCCACCCT |
| Il1b RP | TGTGCTCTGCTTGTGAGGTGCT |
| Oct4 FP | CAGCAGATCACTCACATCGC |
| Oct4 RP | CGCCGGTTACAGAACCATAC |
| Sox2 FP | CCTGGGCGCGGAGTGGAAAC |
| Sox2 RP | CCGCCGCGGCCGGTATTTAT |

Human Real-Time PCR primers are listed in Table 2.

TABLE 2

| | |
|---|---|
| CTCF FP | gggcttgagagctgggttctatt |
| CTCFRP | cttcgactgcatcaccttccatt |
| TBP FP | gtgaacatcatggatcagaacaaca |
| TBP RP | aagatagggattccgggagtcat |
| CDKN2A FP | acattcatgtgggcatttcttg |
| CDKN2A RP | aatgcttgtcatgaagtcgacag |
| IL1A FP | CCAGTGCTGCTGAAGGAGATGCC |
| IL1A RP | CCCTGCCAAGCACACCCAGT |
| IL1B FP | GCTCTCCACCTCCAGGGACAGG |
| 1L1B RP | TGAGGCCCAAGGCCACAGGT |

### Mice

All animal experiments were carried out following the protocols and ethical guidelines, as approved by the Universidad Católica San Antonio de Murcia IACUC and conform to regulatory standards. LMNAG609G mice were generated by Carlos López-Otin at the University of Oviedo, Spain and kindly donated by Brian Kennedy at the Buck Institute. Eight-week-old male C57BL/6J mice were obtained from the Jackson Laboratory. p16-3MR (tri-modality reporter) mice were obtained from the Jackson Laboratory. p16-3MR mice contain functional domains of a synthetic Renilla Luciferase (LUC) monomeric red fluorescent protein (mRFP), and a ganciclovir-sensitive truncated herpes simplex virus 1 thymidine kinase (HSV-TK) under one copy of CDKN2A (p16) promoter. p16-3MR mice show induction of luciferase, consistent with p16 expression during aging. Animals were housed in a specific pathogen free environment and kept under standard conditions with a twelve-hour day/night cycle and access to food and water ad libitum.

Adeno-associated virus (AAV) particles (1×10¹¹ genetic copies (GC)/mouse as specified) of AAV DJ serotype, carrying GFP or CDKN2A promoter-OSK were injected via tail vein in 300 μL PBS into each mouse. Mice were routinely observed for body weight loss and other defects. Mice were sacrificed by CO₂ inhalation or cervical dislocation and blood harvested by cardiac puncture. Immediately tissues were either fixed or flash frozen for H&E and RNA.

### Open Field Test

The baseline activity was measured by placing each mouse individually in the center of a 40×40 cm2 white box with 40 cm high walls for 10 min. The light intensity was 290 lx in the center of the arena. The mouse activity was digitally recorded using a video camera placed 1 m above the center of the arena. The automatic detection of the mouse path was analyzed with the SYGNIS tracker software (SYGNIS). Besides the analysis of the general locomotion, the latency, duration, and the number of visits by the mouse to the inner arena (10×10 cm2) away from the wall were calculated for measuring the anxiety level. The open field test was performed at post-natal day 135.

### IHC Staining

For IHC, tissues were harvested, fixed in 10% neutralized Formalin for 2 days and then stored in 70% ethanol until further processing. H&E staining on paraffin-section was performed following standard protocols.

### Hematopoietic Stem/Progenitor Cell Analysis in Mice

Bone marrow cells were isolated from tibia and femur of mice and were stained with antibodies as detailed below. Antibodies staining was performed for 90 minutes, followed by a PBS wash before analysis. Bone marrow analysis was performed on a FACS AriaII cell sorter (BD Biosciences). Collected data were analyzed with FlowJo software (Tree Star, Ashland, OR). Bone marrow cell type frequencies were calculated from 2×10⁶ live lineage-BM cells per mouse.

Phenotypic cell-surface markers to label hematopoietic stem/progenitor cells (including HSCs, MPPs, LMPPs) were stained with a lineage cocktails (CD4, CD8, B220, Gr-1, TER-119 and CD127), CD34, CD150, CD41, c-Kit, Sca-1, and Flt3).

### RNA-Seq and Data Analysis

Total RNA was extracted with Invitrogen PureLink RNA Mini Kit. Extracted RNA was quantified at OD260 nm with a ND-1000 spectrophotometer (Nanodrop Technology) and quality of the RNA was assessed with the RNA 6000 LabChip kit using a Bioanalyzer 2100 (Agilent Technologies). Poly-A RNA isolation and Library Preparation was done using NEBNext® Poly(A) mRNA Magnetic Isolation Module and NEBNext® Ultra™ II RNA Library Prep Kit for Illumina® as per manufacturer's instruction. Sequencing was performed with Illumina, HiSeq 4000 SR50 for Single-End, 50-cycle. The reads were aligned to the mouse genome (mm8) using TopHat (version 2.0.9) (Trapnell, C., Pachter, L. & Salzberg, S. L. Bioinformatics 25, 1105-1111, 2009) with the default options. After library size normalization using DESeq (Anders, S. & Huber, W. Genome Biol 11, R106, 2010), expression was quantified and expressed in reads/kilobase of transcript per million mapped reads (RPKM) using cufflink (version 2.1.1) (Trapnell, C. et al. Nat Biotechnol 28, 511-515, 2010). Differential expression analysis was performed using the DESeq package with an FDR cutoff of 0.1.

### Data and Materials Availability

The next-generation sequencing data generated in this study are available under accession number: GSE201710 at the NCBI GEO website.

### Example 1—Suppression of Senescence-Associated Secretory Phenotype (SASP) Activity and Partial Reprogramming

Cellular senescence plays a major role during physiological aging and HGPS by both cell-intrinsic and extrinsic programs mediated by SASP (Coppé, J.-P., Desprez, P.-Y., Krtolica, A. & Campisi, J. Annual Review of Pathology: Mechanisms of Disease 5, 99-118, 2010; Kuilman, T. & Peeper, D. S. Nat Rev Cancer 9, 81-94, 2009; Di Mitri, D. et al. Nature 515, 134-137, 2014; Eggert, T. et al. Cancer Cell 30, 533-547, 2016). While senescent cells are thought to be harmful to the organism, they also have beneficial roles in various physiological processes such as wound healing and blockage of fibrosis. For example, continuous or acute elimination of p16^{high} senescent cells disrupts blood-tissue barriers with subsequent liver and perivascular tissue fibrosis and health deterioration (Grosse, L. et al. Cell Metab 32, 87-99 e86, 2020). Furthermore, since general tissue and organ cell composition in an aged organism is dominated by a large pool of senescent cells, depletion of a high number of them might not always be the preferable strategy to prevent further health deterioration. In fact, recent advances have shown a critical role of senescence cells during normal physiology and targeting senescence cells for clearance has shown harmful consequences (Baker, D. J. et al. Nature 479, 232-236 (2011); Reyes, N. S. et al. Science 378, 192-201 (2022)). Therefore, compositions and methods targeting appropriate cell types and/or appropriate cell states, e.g., senescent, aged, somatic, and/or stem cells are needed to target senescent cells in a manner most beneficial to organismal longevity.

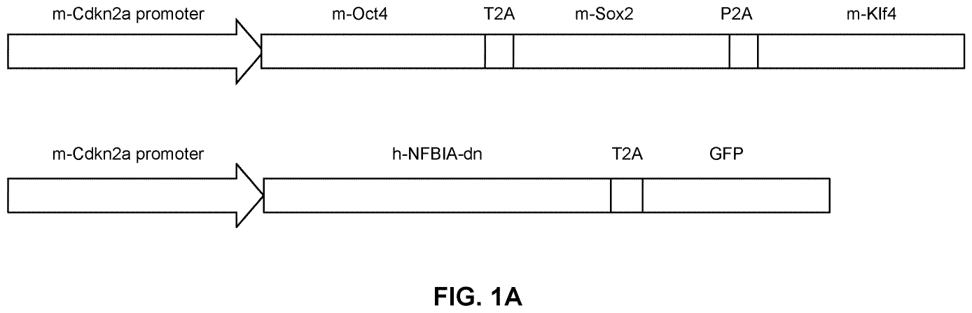

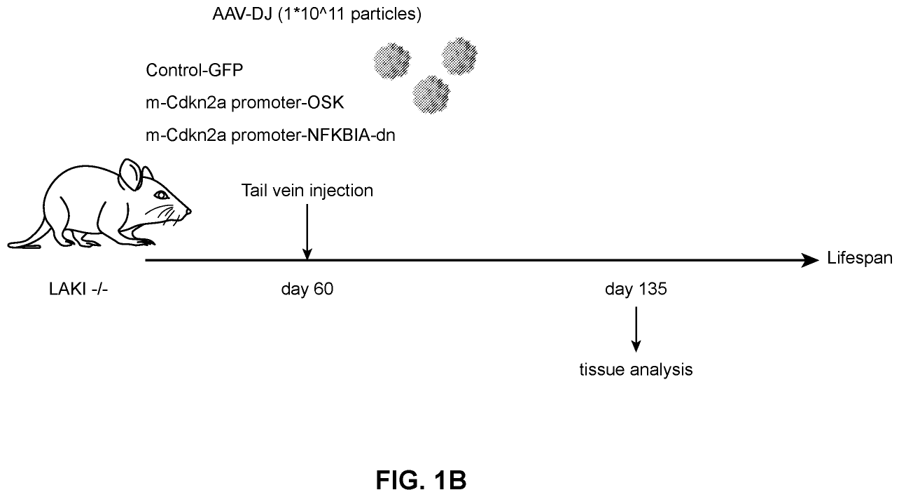

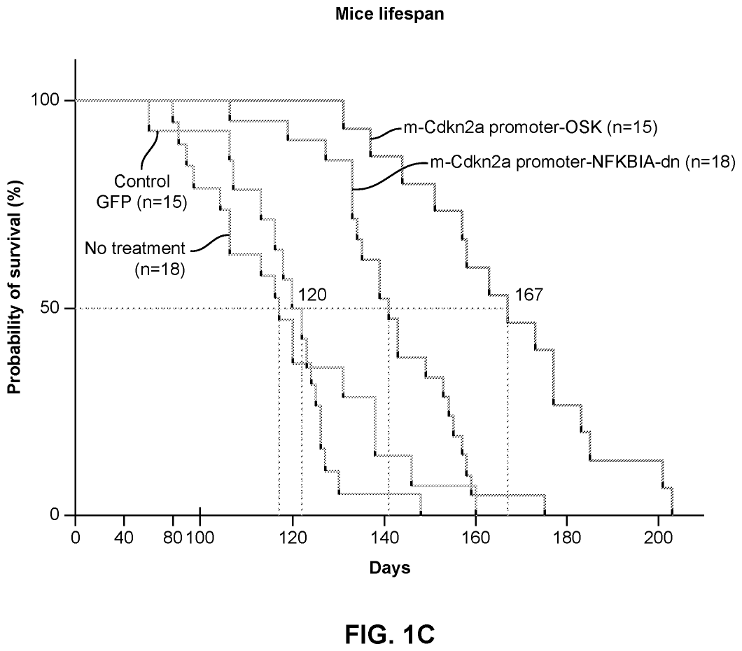

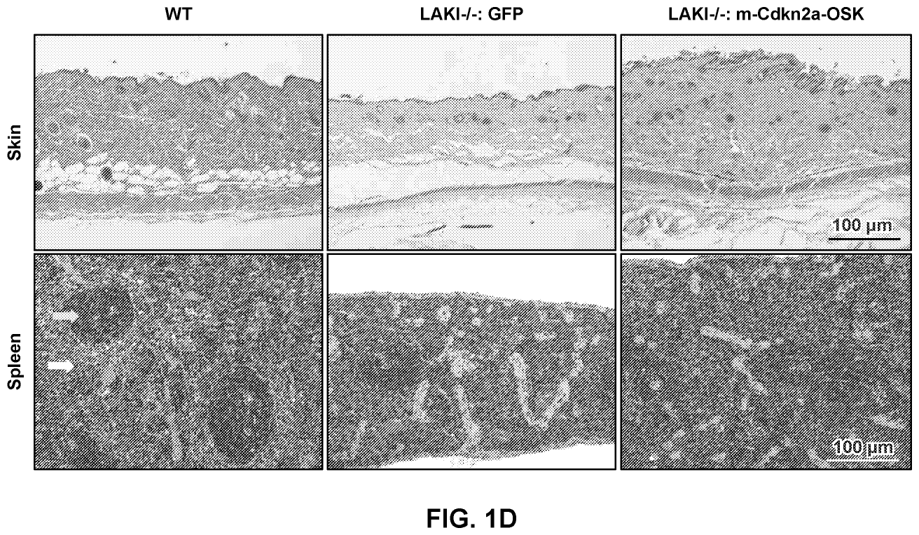

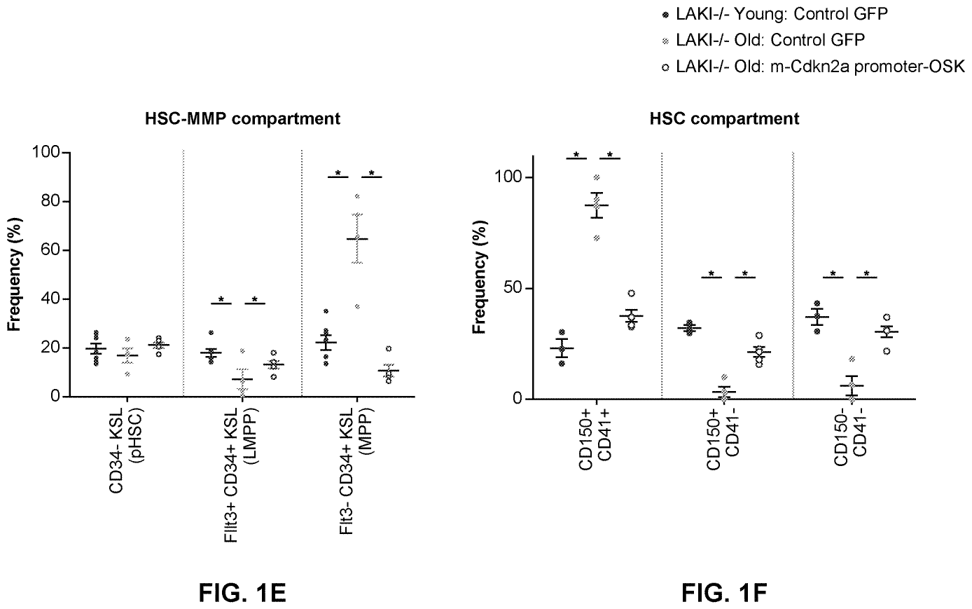

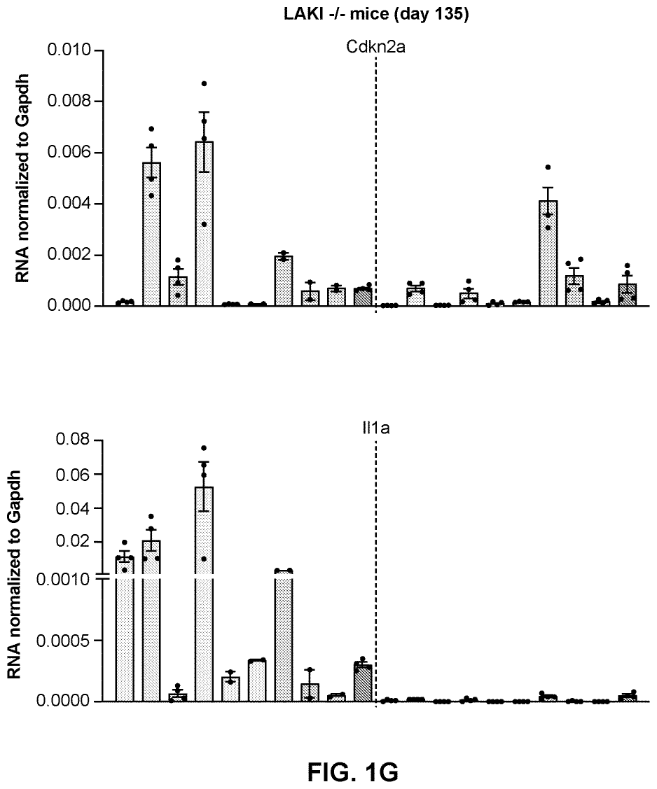

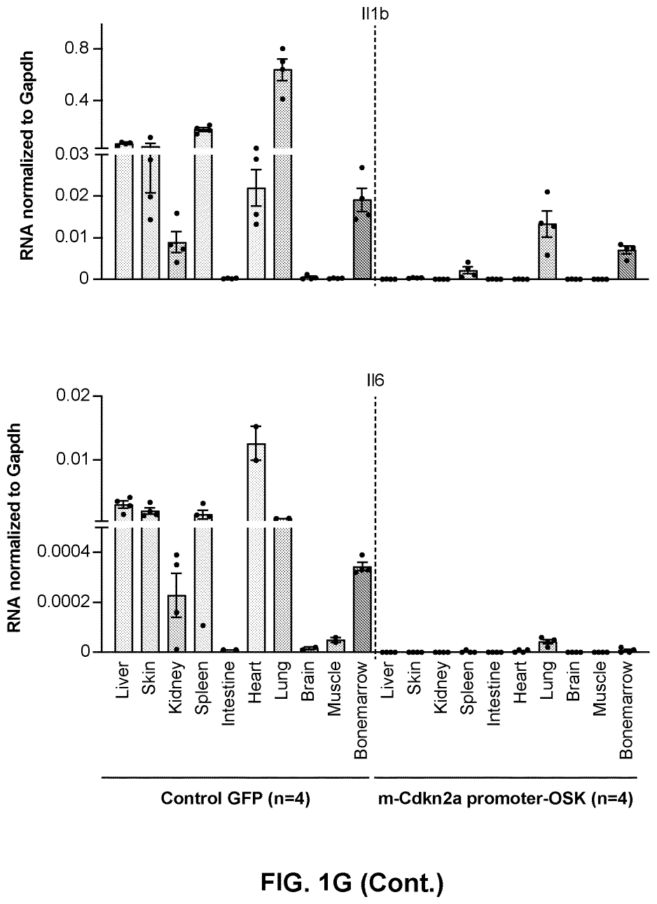

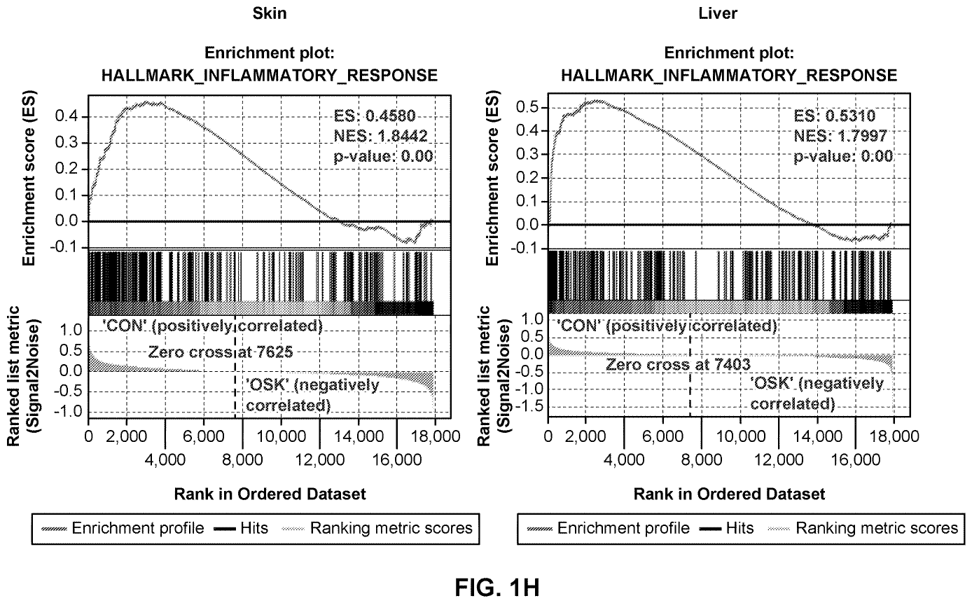

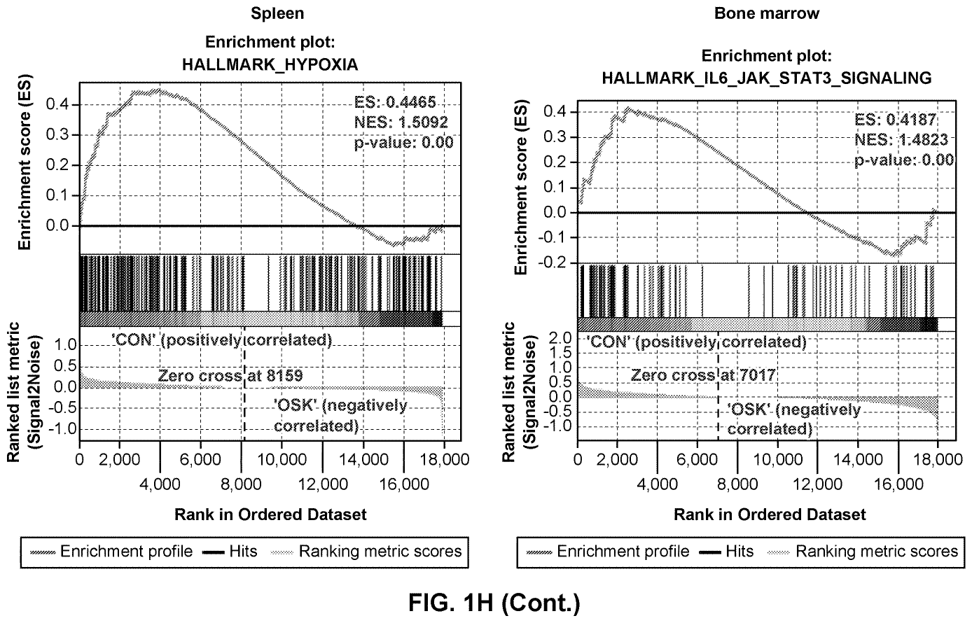

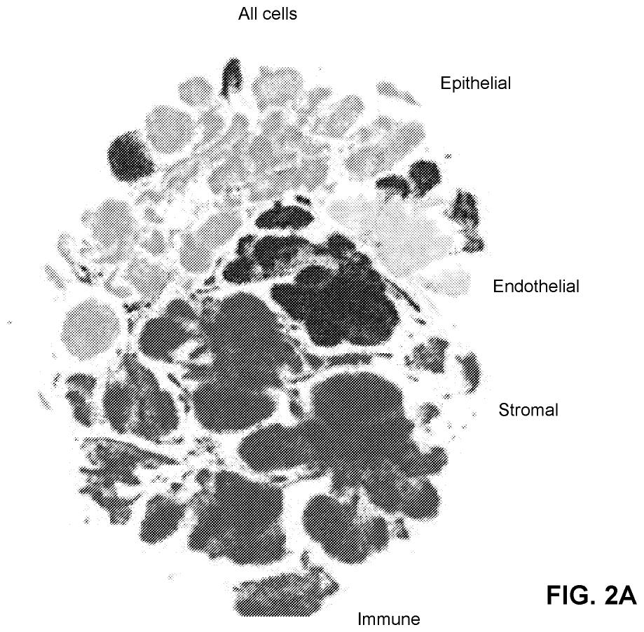

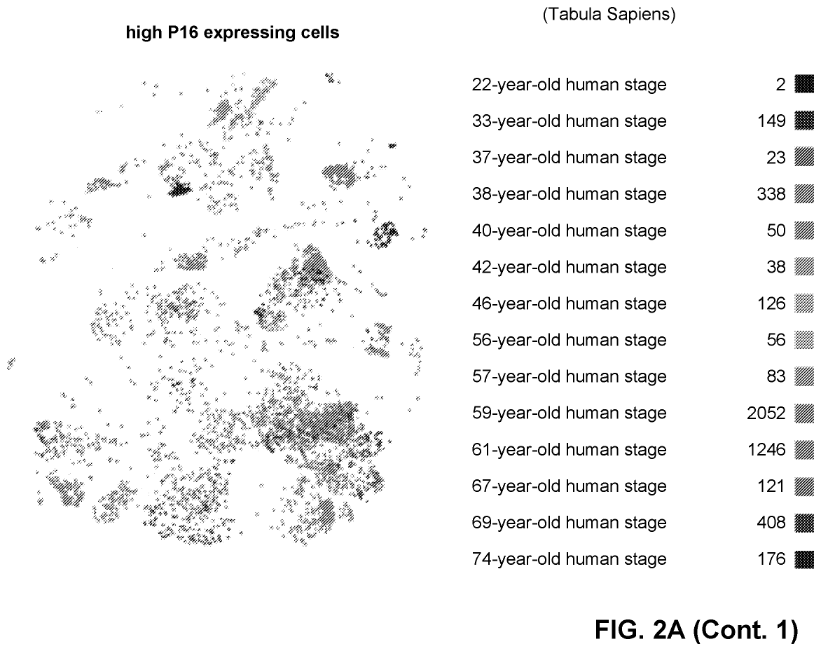

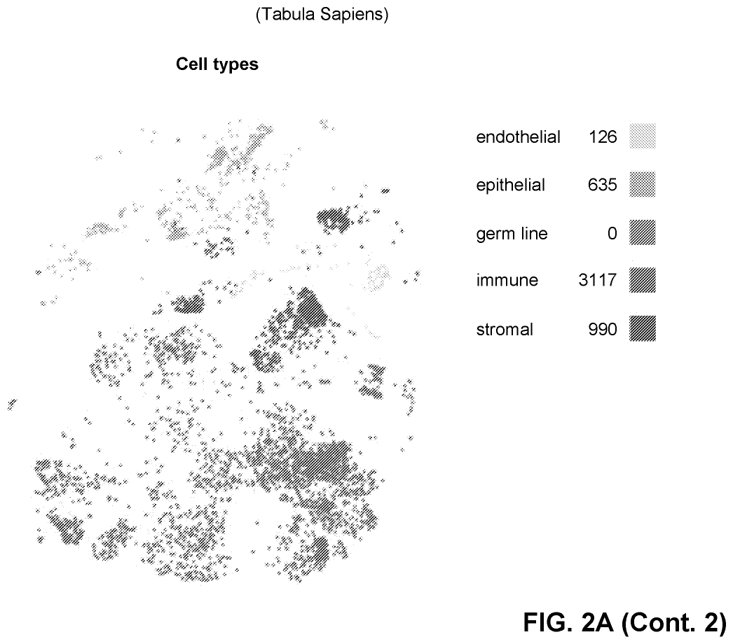

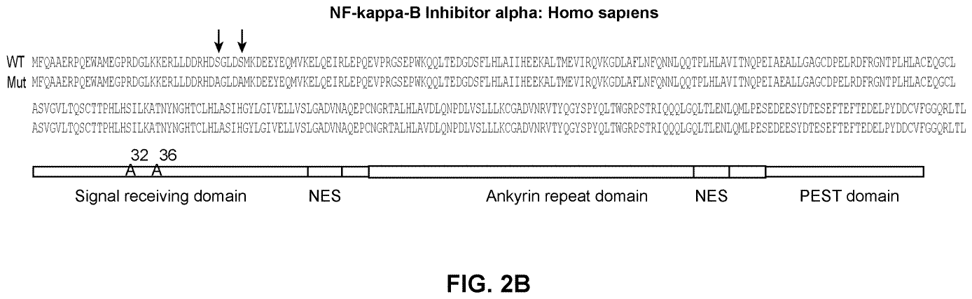

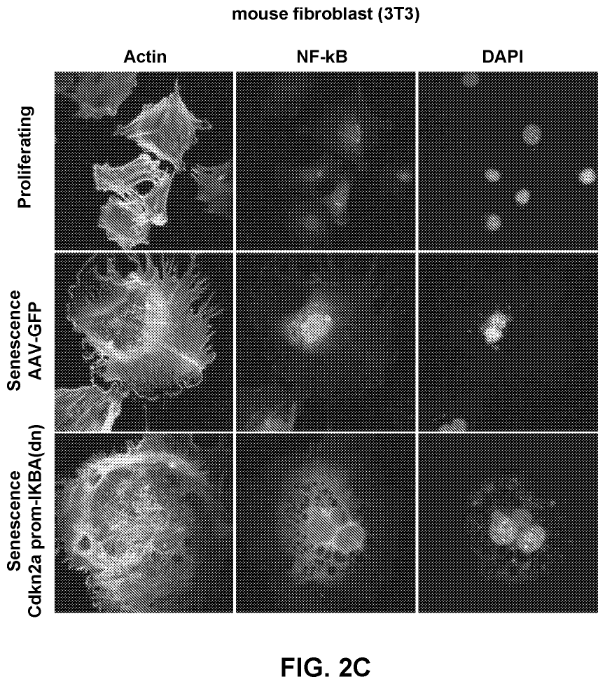

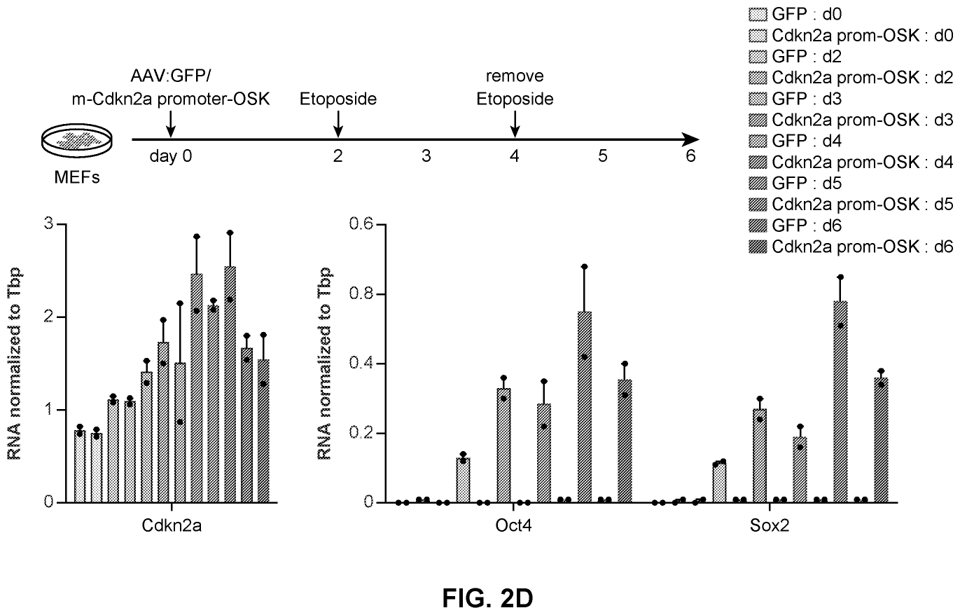

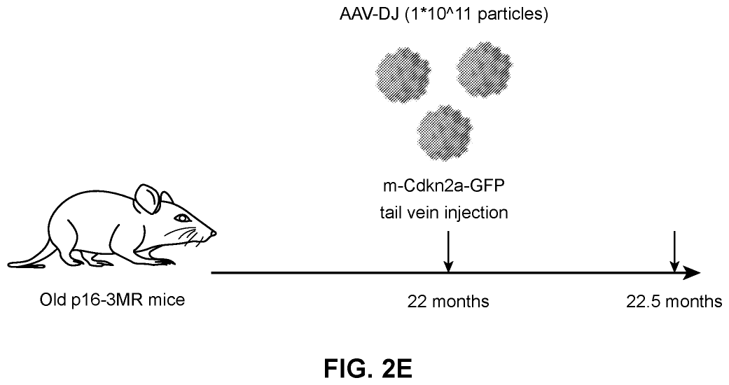

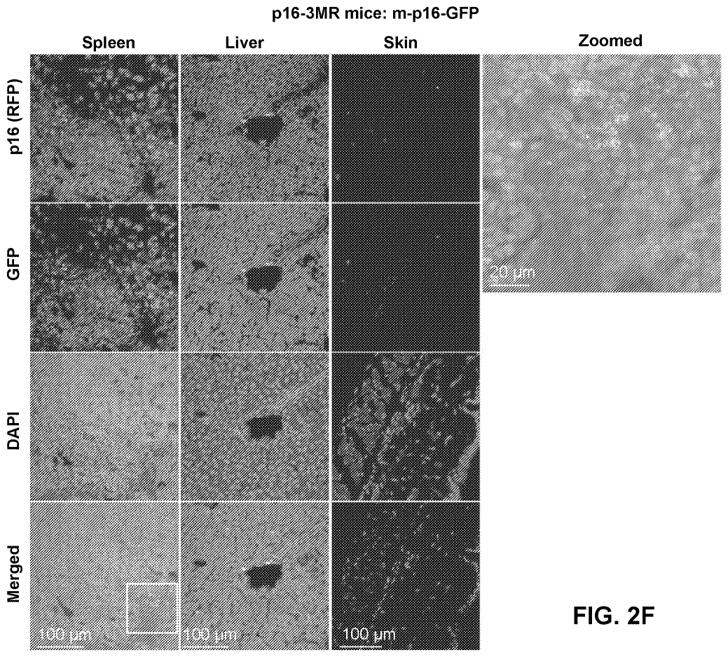

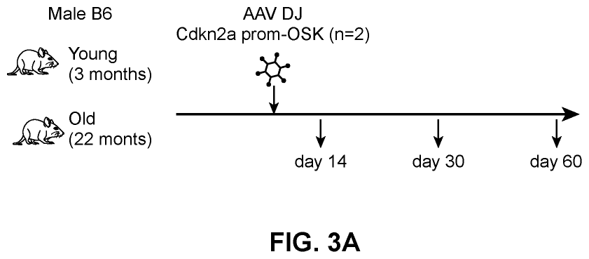

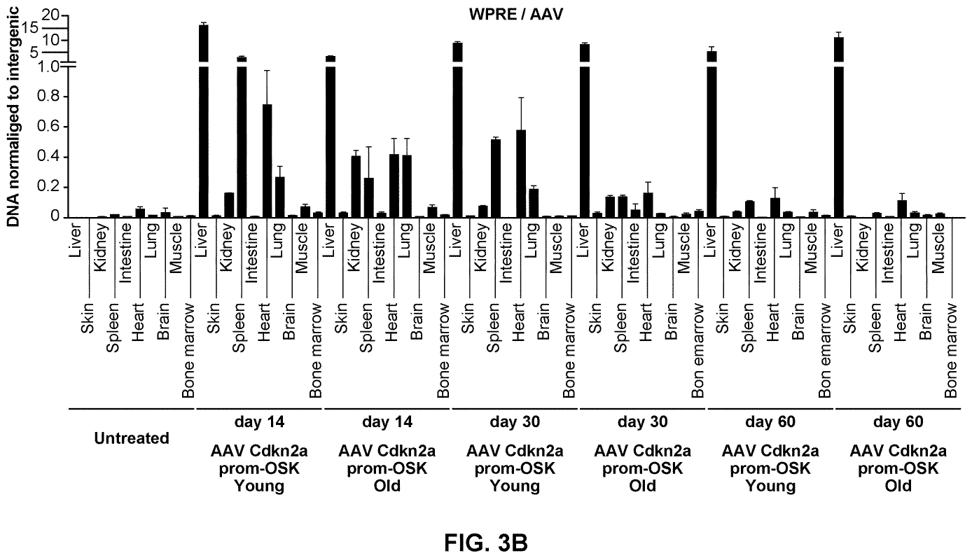

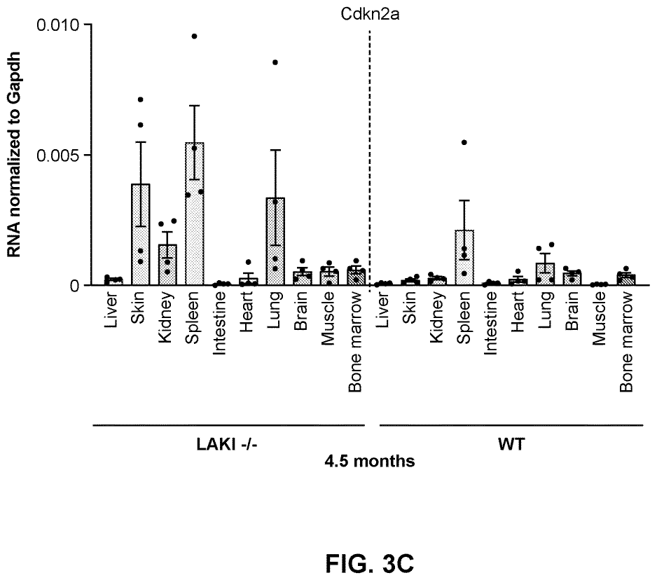

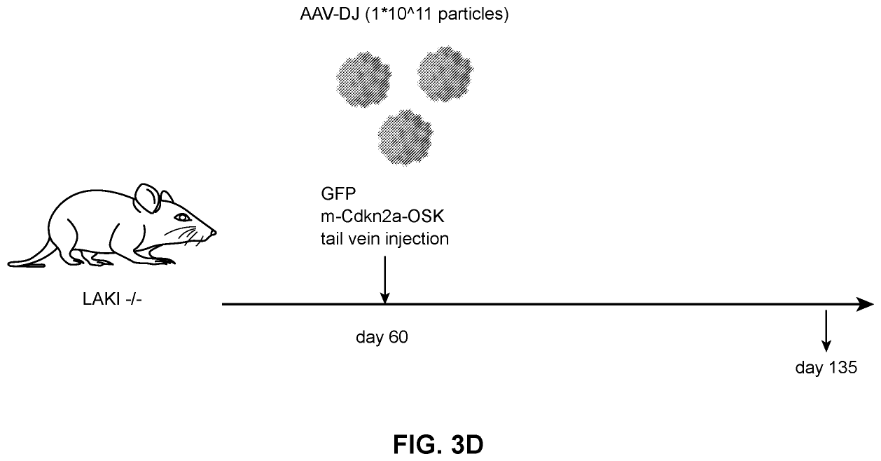

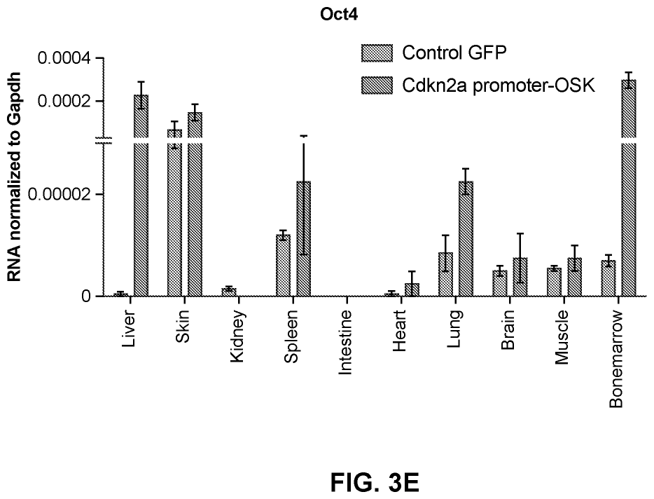

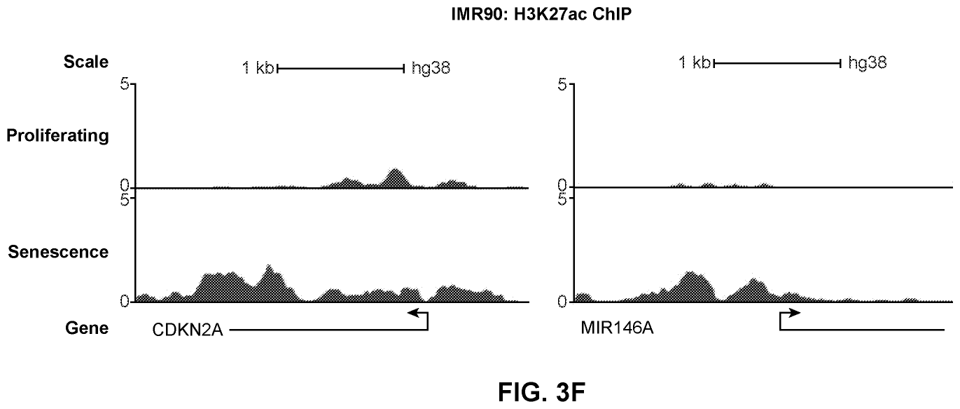

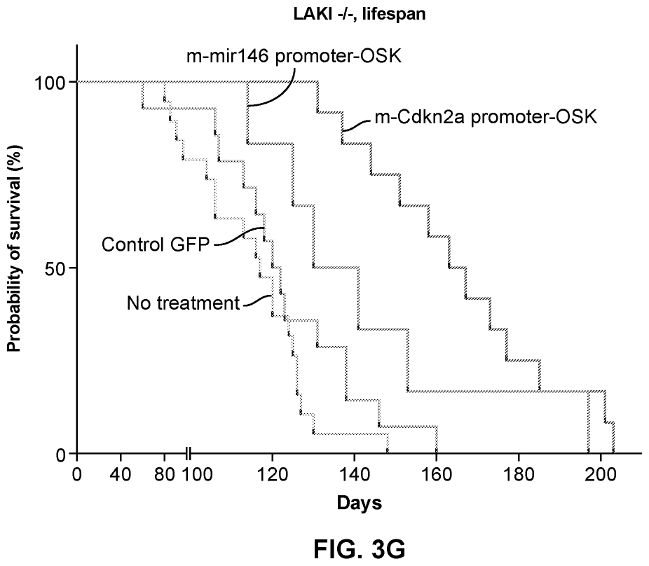

To target senescent cells two approaches were pursued: suppression of SASP activity and partial reprogramming (FIG. 1A, 2B). To suppress SASP, a dominant negative isoform of NFκBIA, which is efficient in blocking nuclear migration of NF-uB, a master transcriptional inducer of SASP was used (FIGS. 1A, 2B, and 2C). For partial reprogramming mediated rejuvenation, the transcription factors Oct4, Sox2 and Klf4 (OSK) were used (FIG. 1A). The transcription factors and dominant negative isoform of NFκBIA were delivered using an adenovirus-associated virus (AAV) vector containing a recombinant AAV genome carrying the genes encoding Oct4, Sox2 and Klf4 (OSK) or the dominant negative isoform of NFκBIA under the control of a Cdkn2a promoter within AAV2 inverted terminal repeats (FIG. 1A). Notably, Cdkn2a is an established marker of senescence (FIGS. 2A and 3F). For example, genome browser tracks derived from ChIP assay for H3K27 acetylation marks performed in proliferative and senescent human lung fibroblasts (IMR90 cells) demonstrated H3K27 acetylation enrichment at the CDKN2A promoter and the miR146 promoter in senescent cells (FIG. 3F). Further, CDKN2A is expressed in various tissues in a premature aging mouse model that has a point mutation in the Lmna gene (LAKI) and manifests symptoms reminiscent of accelerated aging seen in Hutchinson-Gilford Progeria Syndrome (HGPS) patients (FIG. 1G, top panel, and 3C) (See, e.g., Krishnamurthy, J. et al. J Clin Invest 114, 1299-1307, 2004; He, S. & Sharpless, N. E. Cell 169, 1000-1011, 2017; Muss, H. B. et al. Transl Cancer Res 9, 5732-5742, 2020). According to a published single cell transcriptome atlas in humans (Tabula sapiens), higher p16 (CDKN2A) expression is found in older individuals and the high p16 (CDKN2A) expressing cells are mostly immune system related cells (FIG. 2A). The senescent cell specific induction of target genes transduced via AAV particles harboring the respective transgenes under the control of a CDKN2A promoter was confirmed in in vitro cellular systems (FIG. 2C).

Notably, NF-κB is mostly cytoplasmic in young cells and during senescence becomes nuclear and induces SASP. Upon transduction of senescent mouse fibroblasts with CDKN2A promoter-NFKBIA (dn) AAV, NFκB remained in the cytoplasm of senescent cells confirming the functionality of promoter and transgene of the CDKN2A promoter-NFKBIA (dn) AAV vector in vitro (FIG. 2C).

In an in vitro model of DNA damage induced senescence by a 2-day etoposide treatment of mouse embryonic fibroblasts (MEF), DNA damage increased CDKN2A expression in both CDKN2A promoter-OSK AAV treated and Control GFP AAV treated cells. However, CDKN2A promoter-OSK AAV transduction led to an additional increase in CDKN2A expression and an increase in Oct4 and Sox2 expression in CDKN2A promoter-OSK AAV transduced cells confirming the functionality of the promoter and transgene of the CDKN2A promoter-OSK AAV vector in vitro (FIG. 2D).

### Example 2—Cellular Reprogramming of p16 (CDKN2A)-Expressing Cells in a p16-Marker Expression Mouse Model In Vivo

To better analyze the fate of senescent cells undergoing cellular reprogramming in vivo, a mouse model containing functional domains of a synthetic Renilla Luciferase (LUC) monomeric red fluorescent protein (mRFP) and a ganciclovir-sensitive truncated herpes simplex virus 1 thymidine kinase (HSV-TK) under one copy of a p16 promoter (p16-3MR tri-modality reporter mouse) was used (FIG. 2E, see also Demaria, M. et al. Dev Cell 31, 722-733, 2014). For in vivo studies, the AAV-DJ serotype was chosen due its broad organ tropism in mice (FIGS. 3A and 3B).

A single dose of m-CDKN2A promoter-GFP AAV-DJ was injected into old p16-3MR mice (22 months old) and the RFP and GFP signals assessed 15 days post injection in various organs (FIG. 2F). The spleen of old 3MR mice showed high levels of RFP positive cells indicating high levels of senescent cells. Co-expression of GFP and RFP in spleen cells demonstrated m-CDKN2A promoter-GFP AAV-DJ-mediated GFP expression in senescent cells (FIG. 2F, zoomed image, yellow color).

In the liver, even though AAV-DJ successfully transduced hepatocytes, few RFP expressing hepatocytes and no GFP/RFP co-expression were observed indicating low amounts of senescent cells in the liver (FIG. 2F; the few bright color dots in the liver images did not overlay with DAPI nuclear stains and were most likely caused by background fluorescence). Overall, these studies confirmed the successful targeting of p16-expressing cells using the CDKN2A promoter-GFP AAV-DJ vectors.

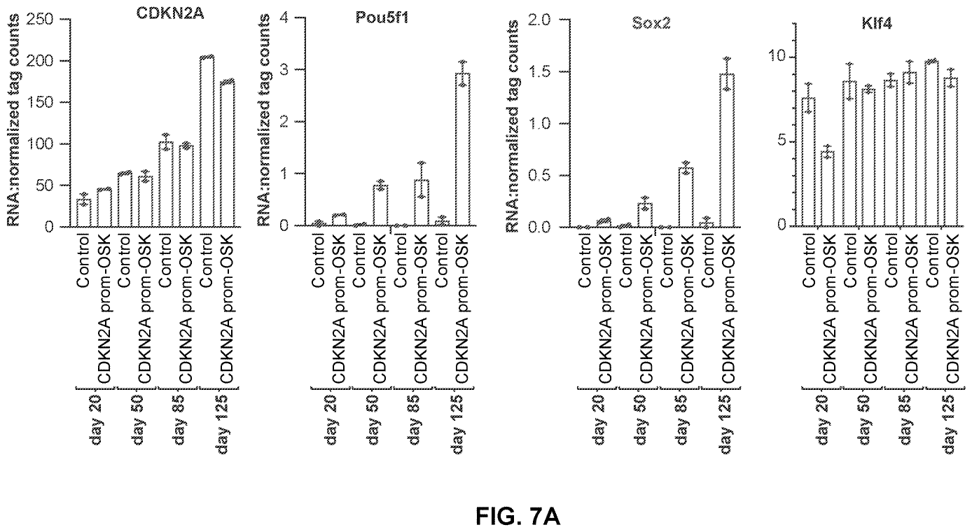

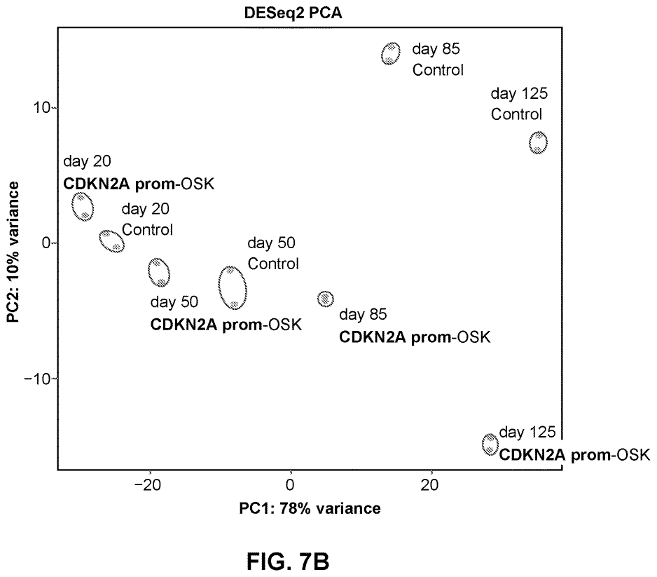

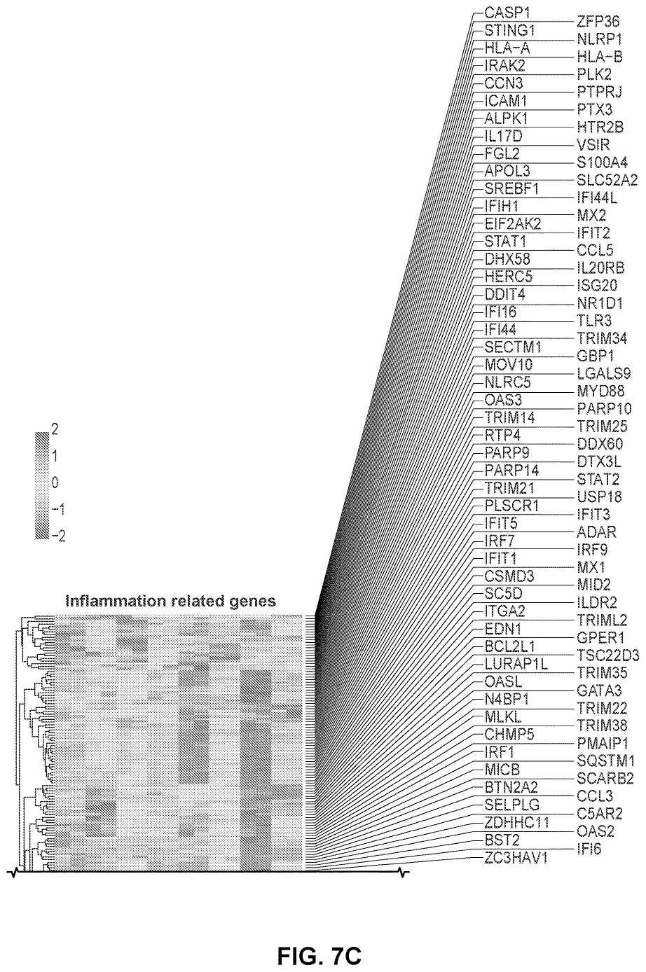

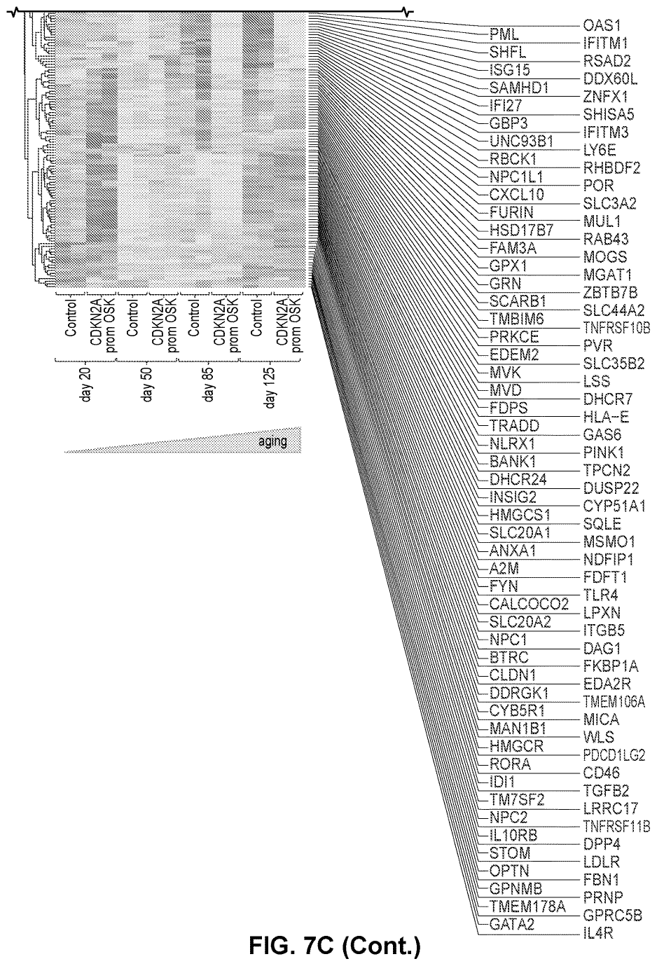

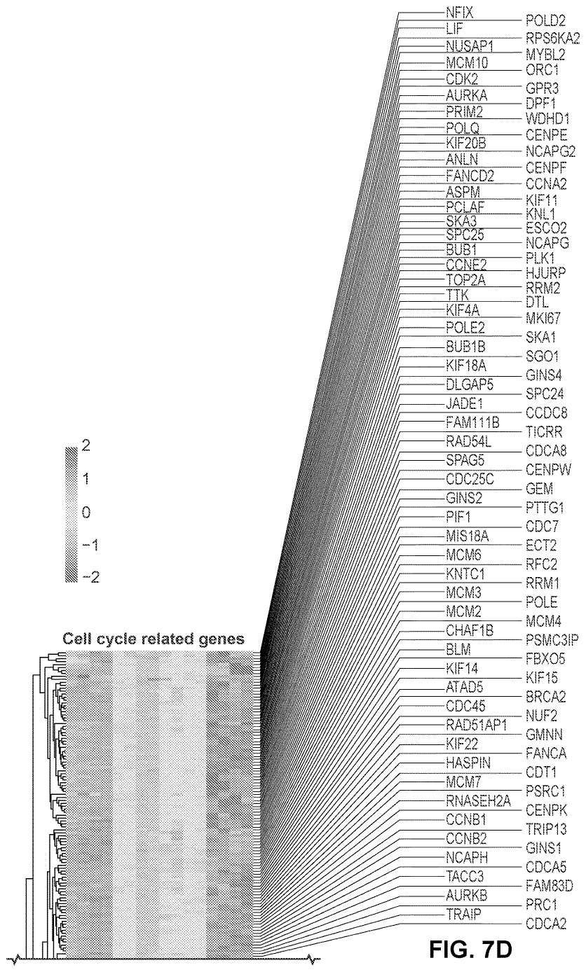

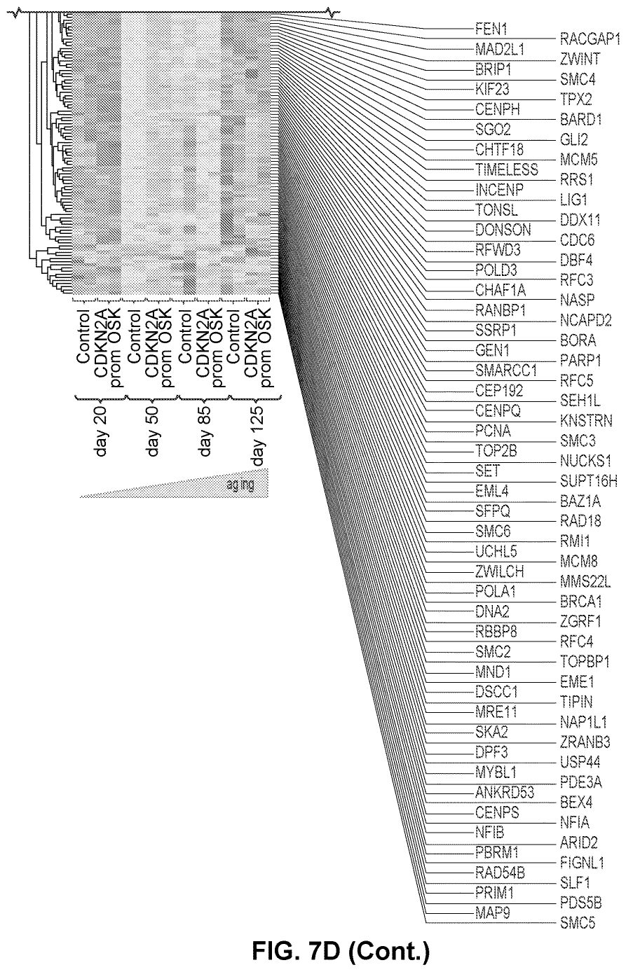

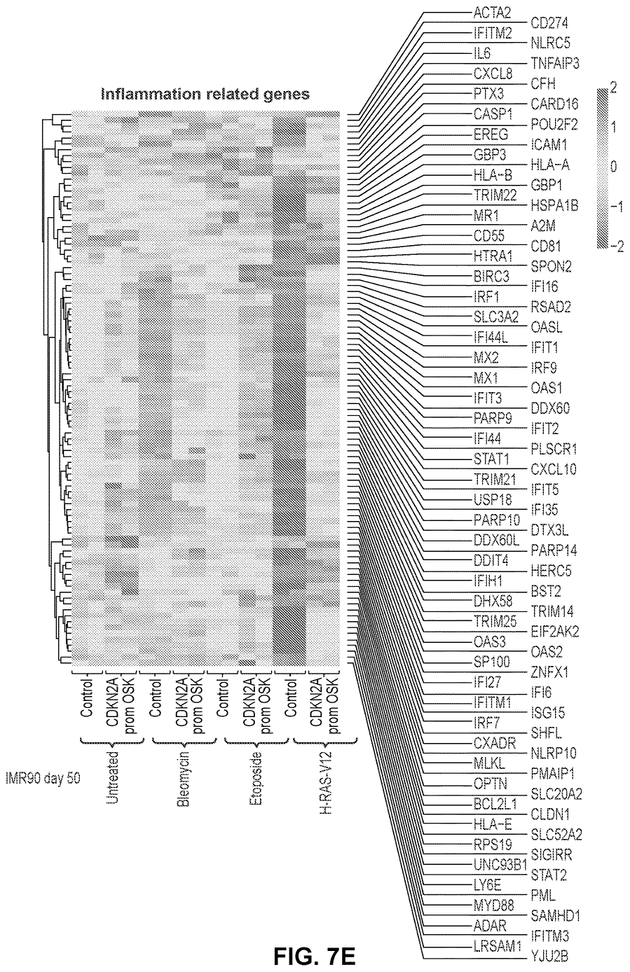

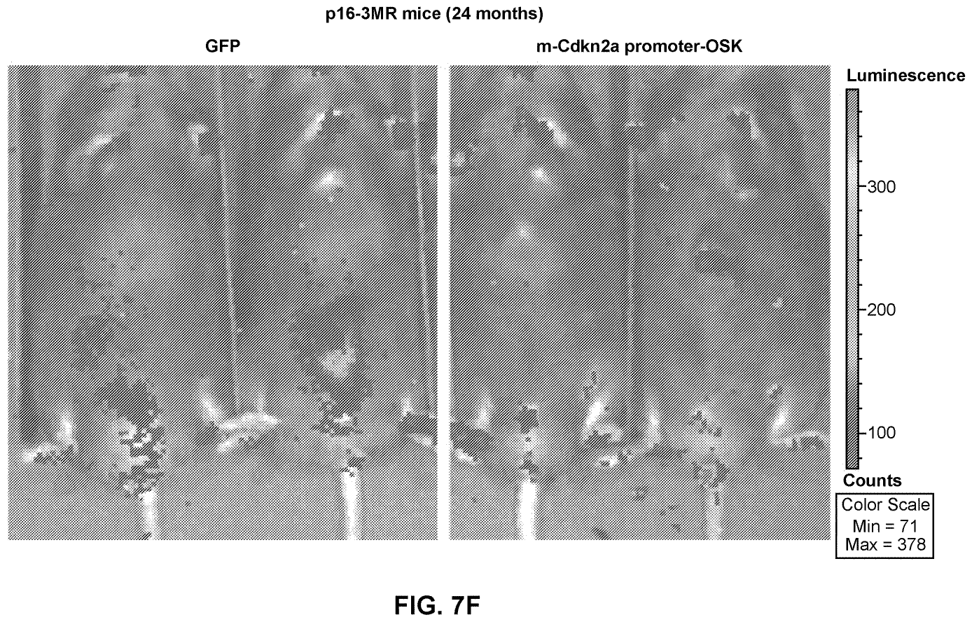

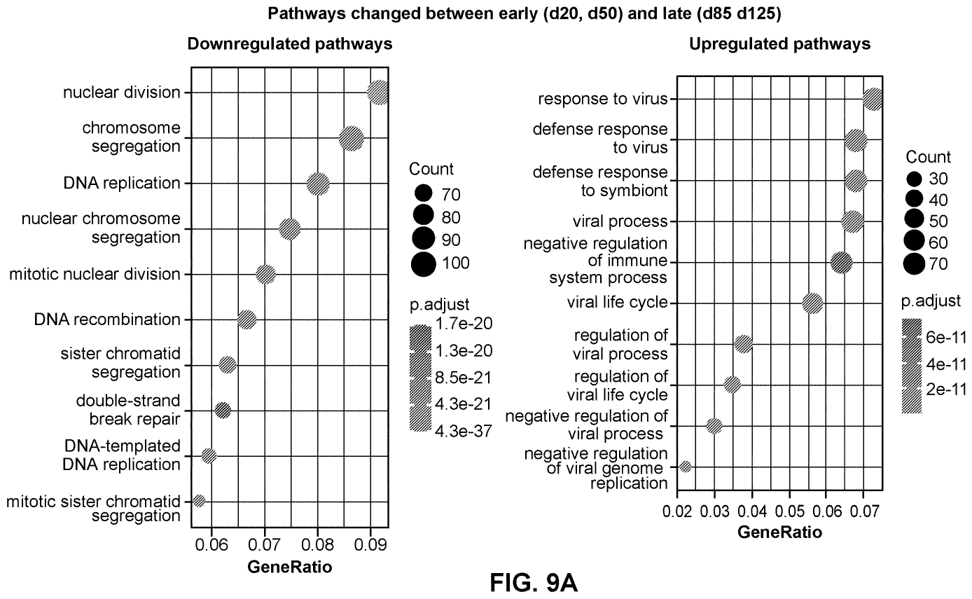

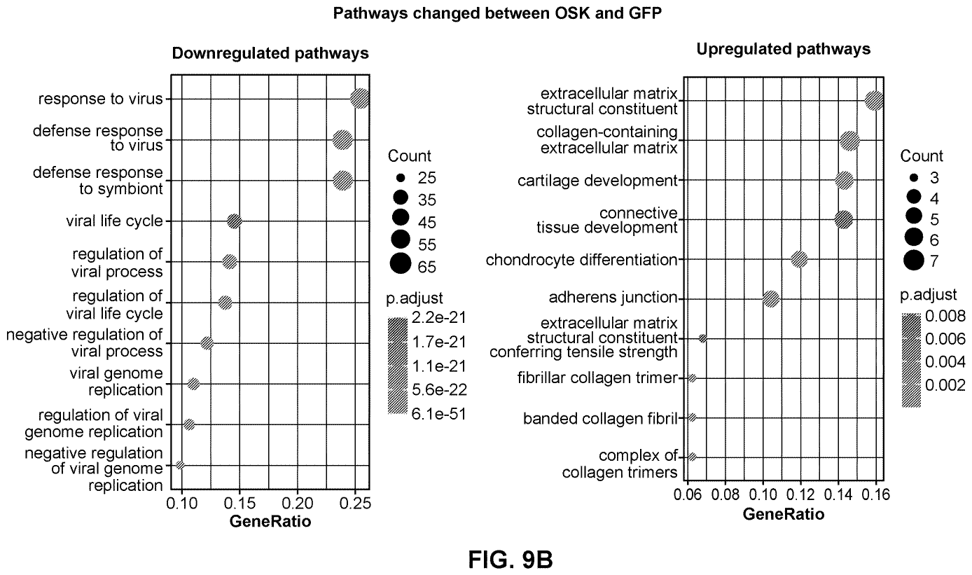

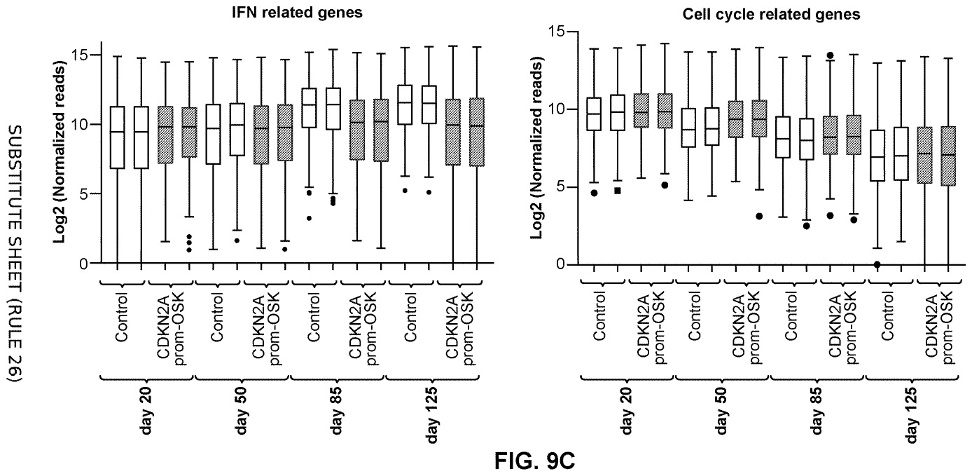

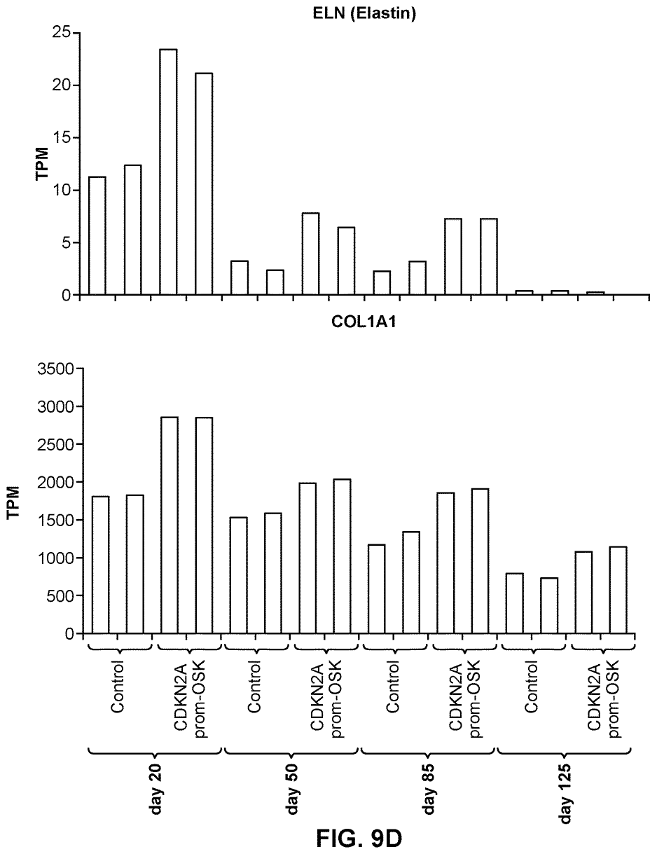

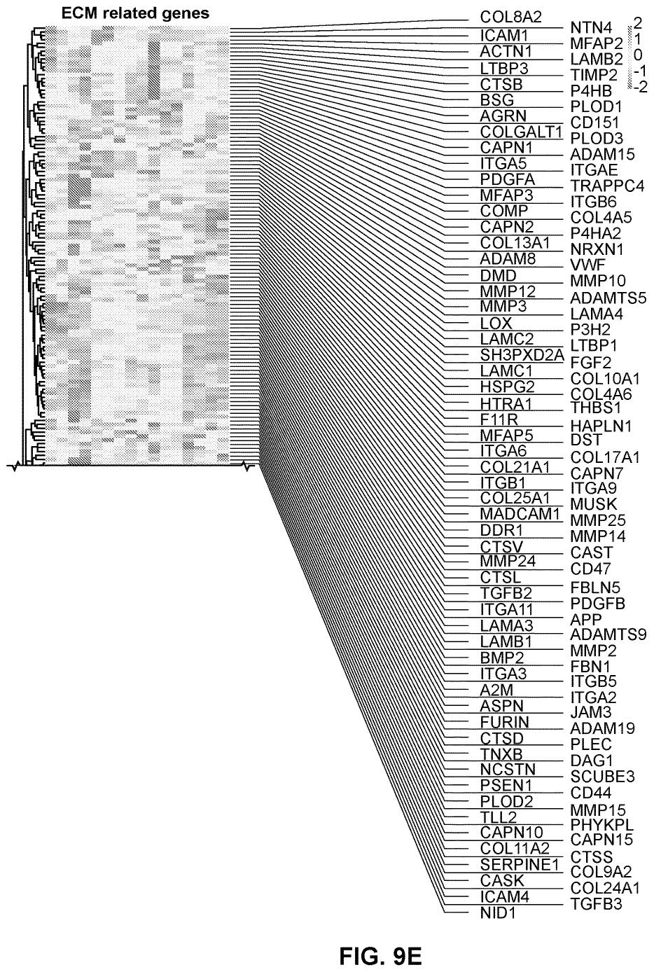

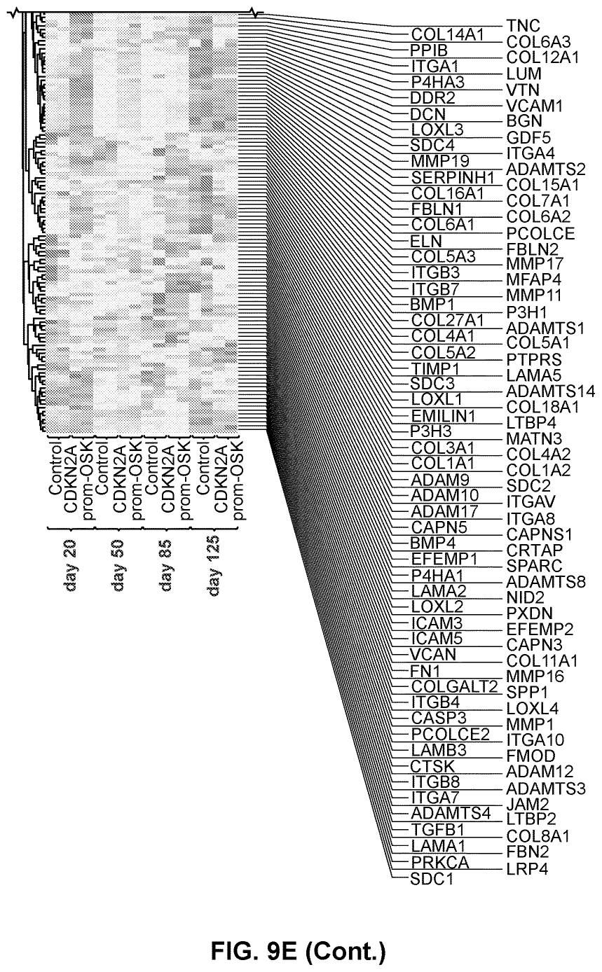

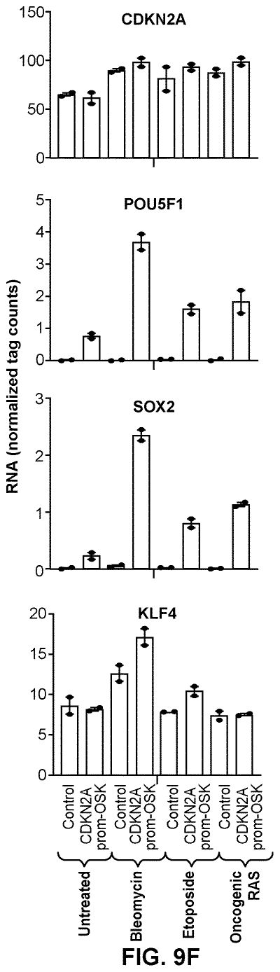

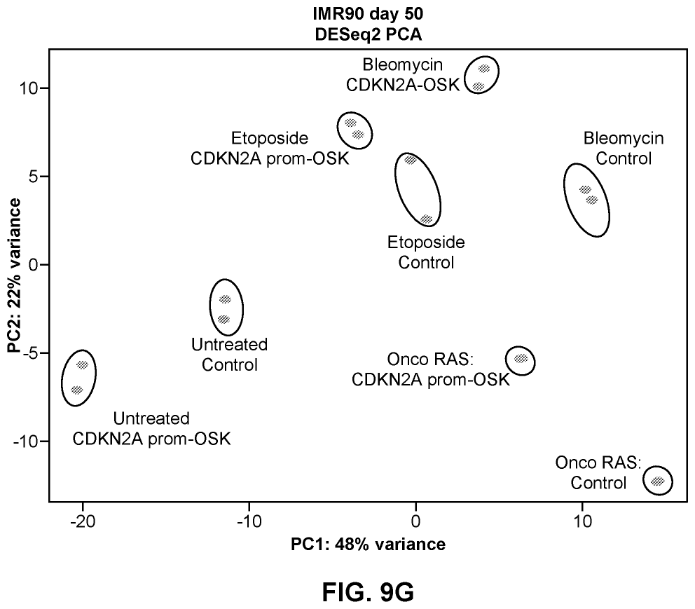

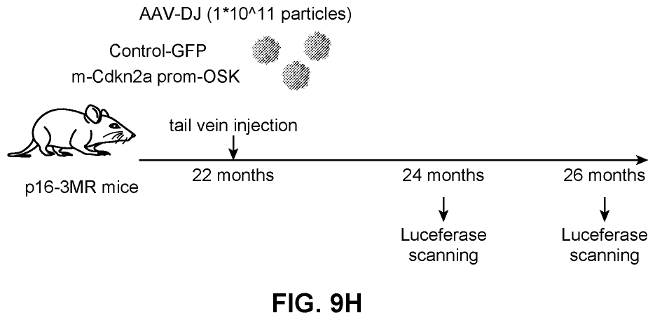

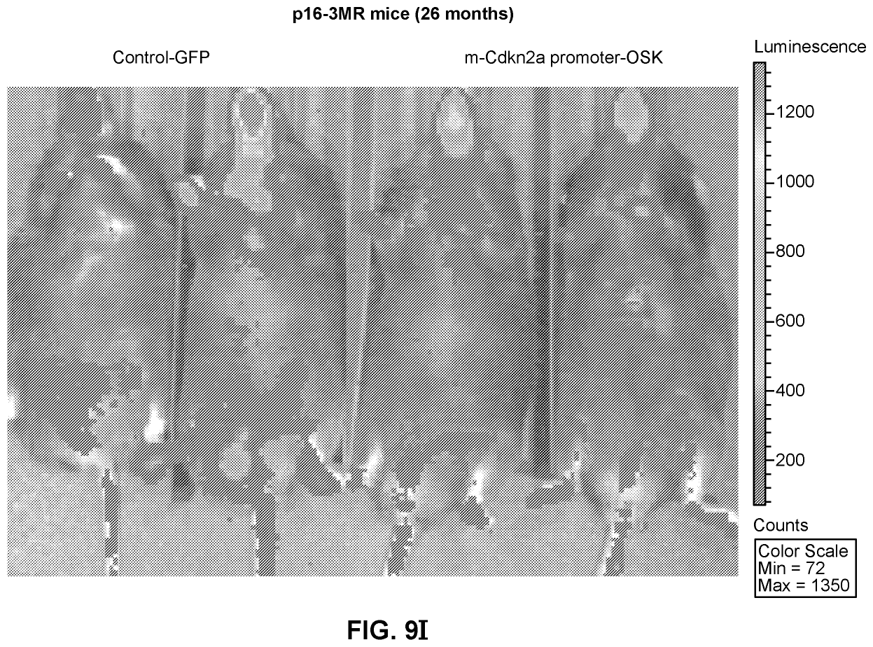

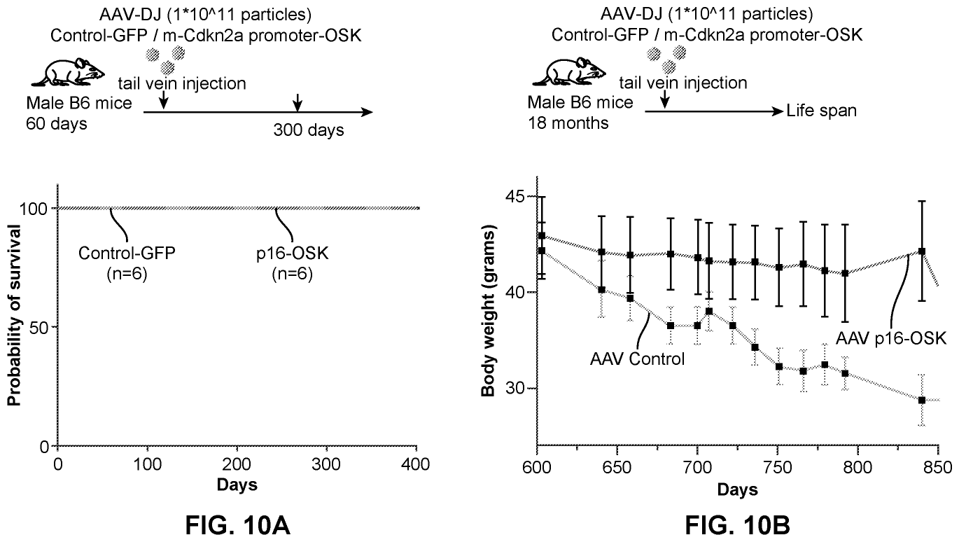

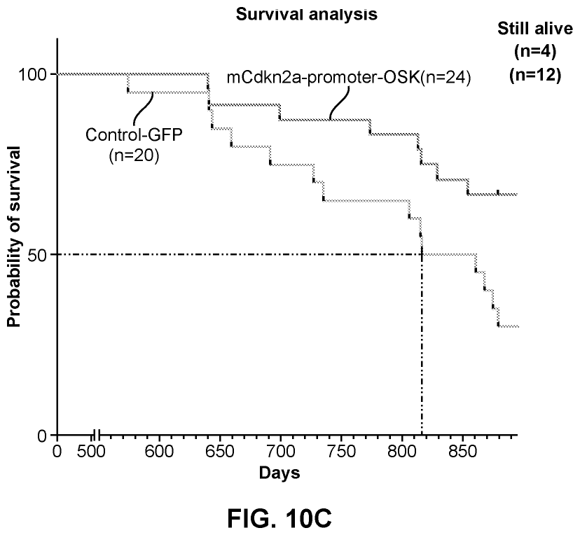

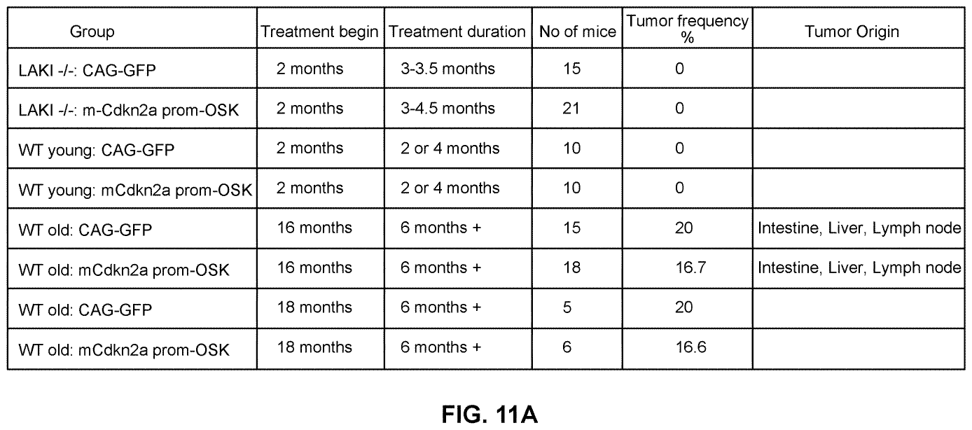

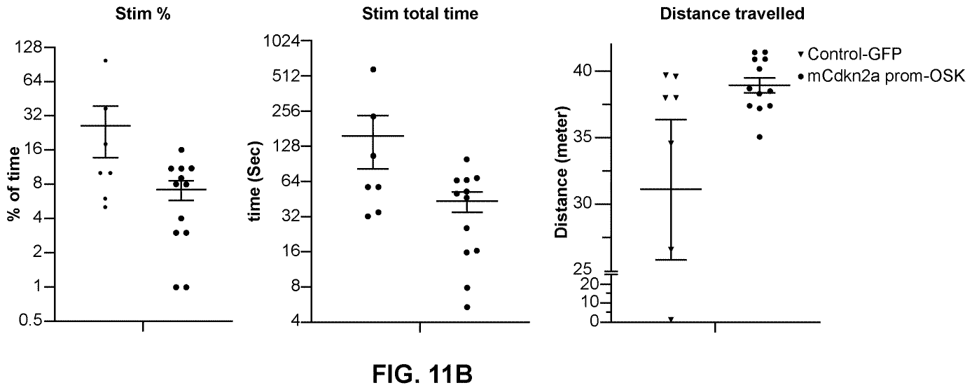

To analyze the fate of senescent cells undergoing cellular reprogramming in vivo, old p16-3MR mice (22 months old) were injected with m-CDKN2A promoter-OSK AAV-DJ and GFP AAV-DJ control. The CDKN2A promoter-OSK AAV-DJ treated p16-3MR mice demonstrated a reduction in luciferase expression at 24 and 26 months (2- and 4-months post CDKN2A promoter-OSK AAV-DJ injection) compared to constitutive promoter-GFP AAV-DJ injected control mice indicating a reduction in senescent cells upon CDKN2A promoter-OSK AAV-DJ treatment (FIGS. 7F, 9G and 9H). These results indicated that CDKN2A promoter driven OSK expression successfully reduced the number of senescent cells in CDKN2A promoter-3MR mice in vivo.

### Example 3—Effect of Senescent Cell Specific Interventions on Organismal Lifespan

To test whether senescent cell specific intervention could improve age-related phenotypes and extend organismal lifespan, the premature aging LAKI−/− mouse model was used (FIG. 1B, 3D). Eight week old LAKI−/− mice were injected with a single dose of AAV-DJ harboring CAG-GFP, m-CDKN2A promoter-OSK or m-CDKN2A promoter-NFκBIA-dn (FIG. 1B). Blockage of SASP activity in senescent cells with m-CDKN2A promoter-NFκBIA-dn significantly improved median lifespan (Control: 120 days vs. CDKN2A promoter-NFκBIA: 140 days, around 17%). Partial reprogramming via OSK expression strongly improved both median (around 40%) and maximal (around 32%) lifespan in LAKI−/− mice (FIG. 1C). Oct4 expression in various tissues following m-CDKN2A promoter-OSK AAV-DJ was confirmed (FIG. 3E). Different senescent promoters like mir146a are known to be more sensitive and dynamic in response to senescence/SASP (Kang, C. et al., Science 349, aaa5612 (2015) (FIG. 3F). However, LAKI−/− mice transduced with m-mir146 OSK AAV-DJ showed a weaker improvement in health and lifespan as compared to CDKN2A promoter driven OSK expression (FIG. 3G).

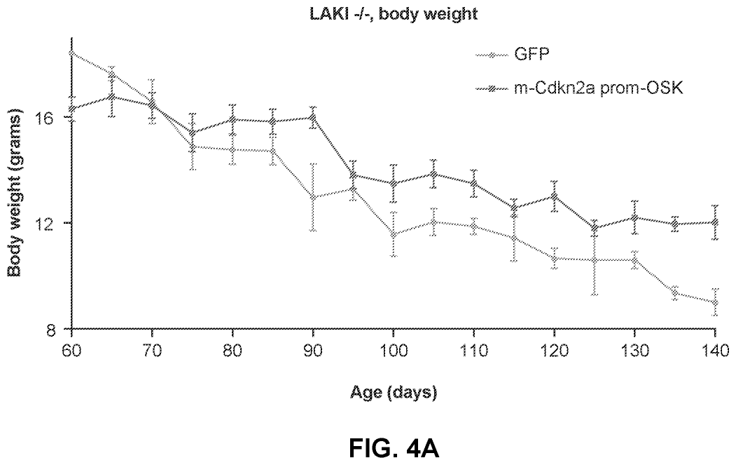

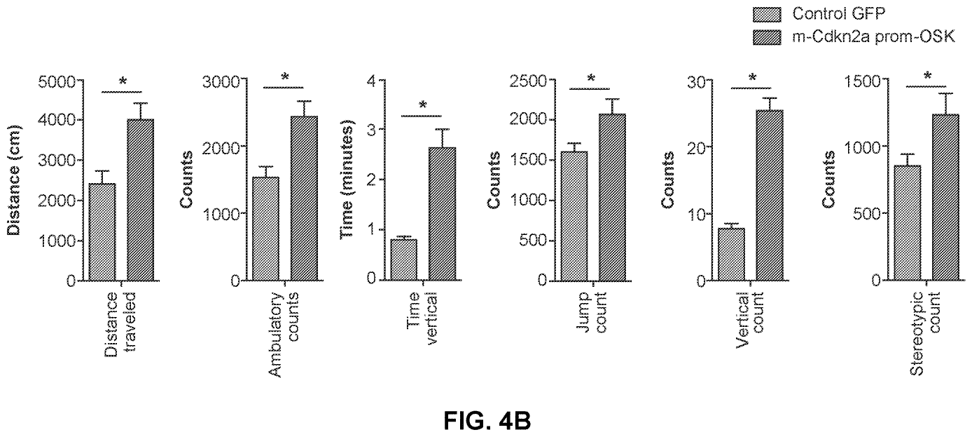

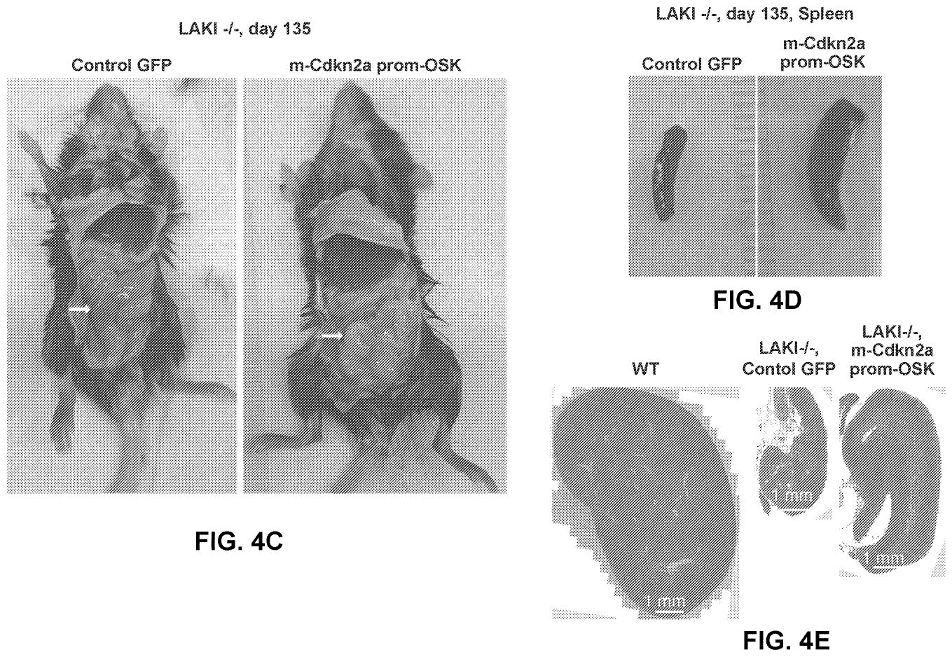

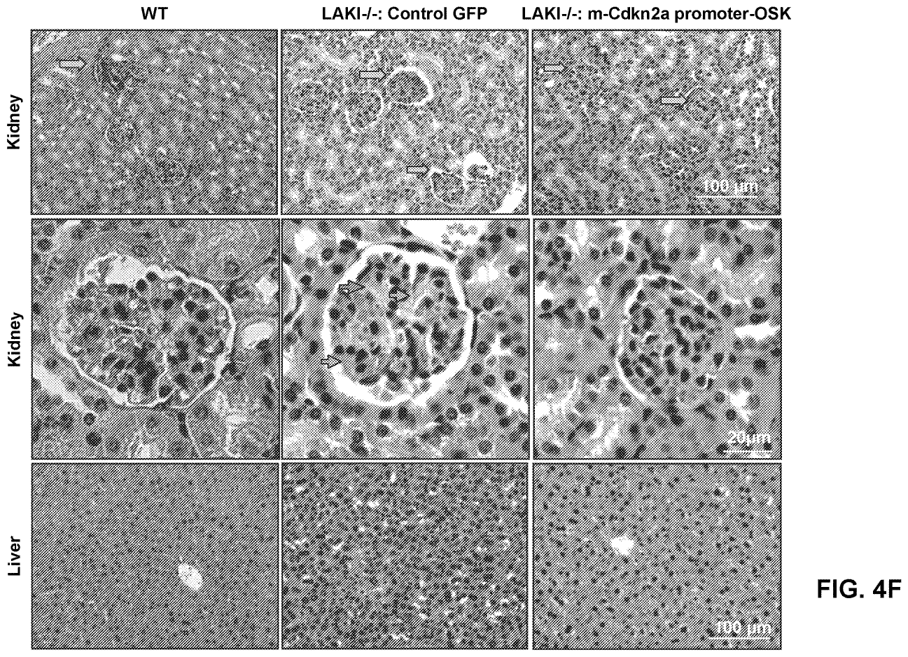

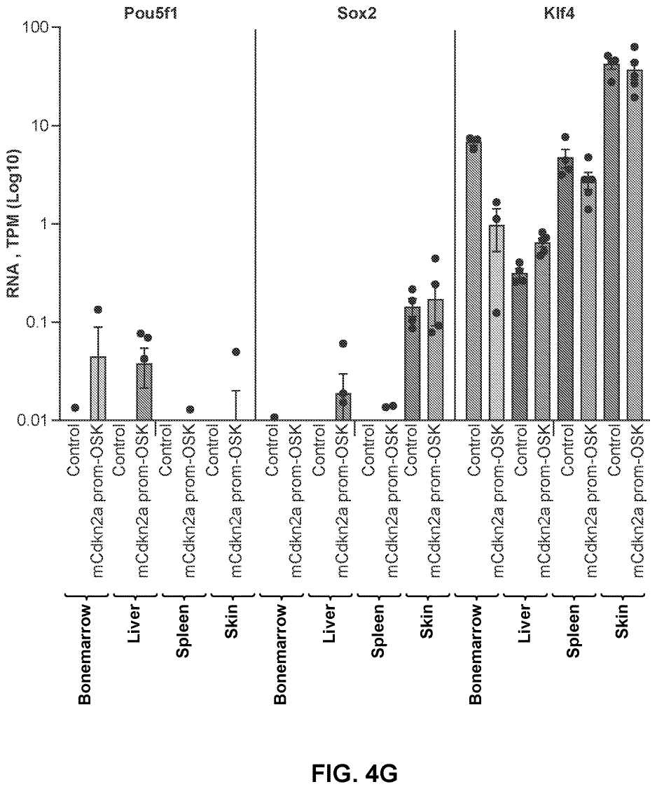

CDKN2A promoter-OSK expression significantly prevented the progressive loss of body weight characteristic of LAKI−/− mice and improved overall physical fitness and activity, which may have been due to systemic physiological improvements (FIGS. 4A and 4B). Detailed necropsy analyses performed at 135 days of age revealed gross improvement in the appearance of the gastrointestinal tract (reduced mega colon/mega cecum with hardened fecal matter) and reduced splenic atrophy in CDKN2A promoter-OSK LAKI−/− mice (FIGS. 4C and 4D). In many of the organs including skin, liver, spleen and kidneys, morphological and histological changes were improved in CDKN2A promoter-OSK AAV-DJ treated LAKI−/− mice compared to GFP AAV-DJ treated control mice (FIGS. 1D, 4E, and 4F). For example, increased epidermal and dermal thickness, a reduced mesangial area in the kidney, reduced kidney atrophy, and increased structural arrangement of hepatocytes in the liver were observed upon CDKN2A promoter-OSK (FIGS. 1D, 4E, and 4F). Furthermore, macroscopic involution of the spleen and lymphoid depletion of the white pulp visualized by the reduced size of germinal centers was significantly rescued in the spleen of LAKI−/− mice after CDKN2A promoter-OSK (FIG. 1D).

Although, the majority of p16 high expressing cells are vascular endothelial cells, macrophages, and adipocytes, see, e.g., Grosse, L., et al., Defined p16 (High) Senescent Cell Types Are Indispensable for Mouse Healthspan. Cell Metab 32, 87-99 e86, 2020), expression of OSK in senescent cells of LAKI−/− mice strongly reduced the overall inflammatory response in a broader range of organs (FIGS. 1G and 1H). Notably, administration of CDKN2A promoter-OSK to LAKI−/− mice reduced CDKN2A/p16 expression in the treated mice indicating a successful reduction of the senescence marker p16 upon CDKN2A/p16 promoter-OSK treatment. Analysis of Oct4 and Sox2 transgene expression in different organs showed a more pronounced expression in some tissues including bone marrow, indicating a paracrine mode of signaling driving the overall improvement of various organs homeostasis and suppression of inflammatory response in LAKI−/− mice (FIG. 4G).

### Example 4—Reversal of Senescence in a Premature Aging Mouse Model

As aging progresses, immune senescence plays a critical role in driving aging in diverse organs (Lee, K. A., Flores, R. R., Jang, I. H., Saathoff, A., Robbins, P. D., Immune Senescence, Immunosenescence and Aging. wFront Aging 3, 900028, 2022). For example, in aging bone marrow, HSCs have decreased homing abilities, decreased per-cell repopulating self-renewal activity, are biased towards myeloid differentiation, and show increased apoptosis with stress, which altogether drive age associated pathology (Esplin, B. L. et al. J Immunol 186, 5367-5375, 2011; Schuettpelz, L. G. & Link, D. C. Front Immunol 4, 204, 2013; Huggins, C. J. et al. Mol Cell Biol 33, 3242-3258, 2013). In the spleen, with age there are disturbances in the functional compartments (red and white pulp) that consist of specialized populations of macrophages, B cells, and stromal cells etc., which are involved in the initial response to blood-borne pathogens (Junt, T., Scandella, E., Ludewig, B., Form follows function: lymphoid tissue microarchitecture in antimicrobial immune defence. Nat Rev Immunol 8, 764-775, 2008).

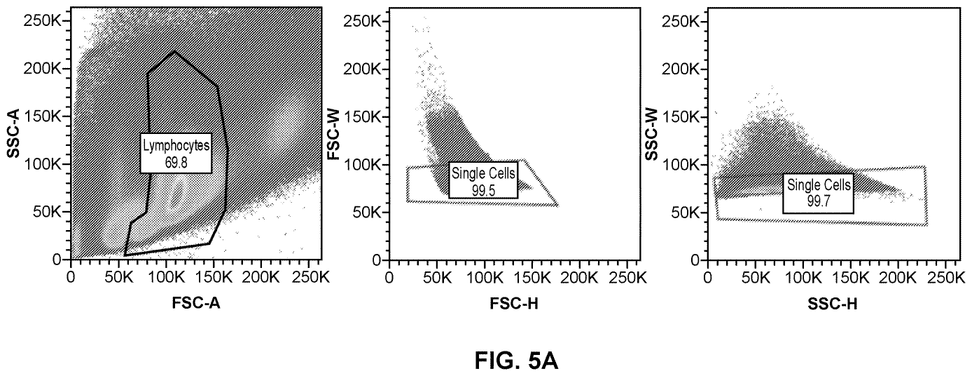

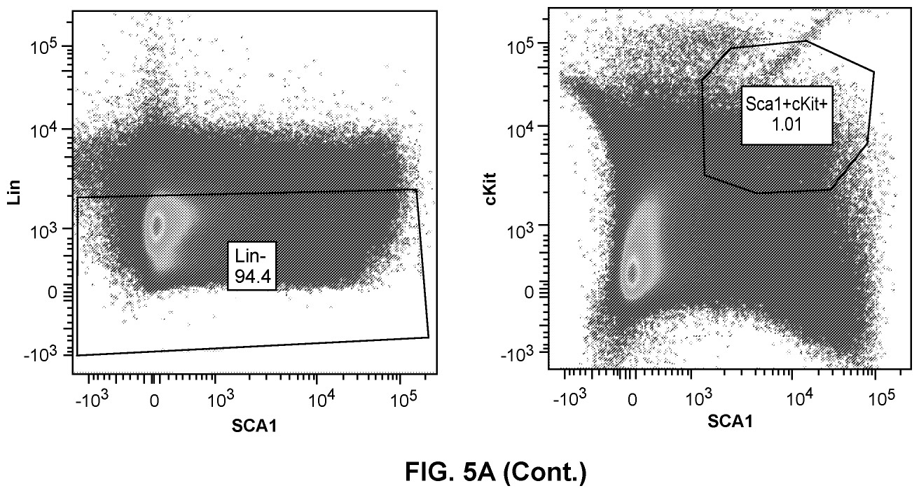

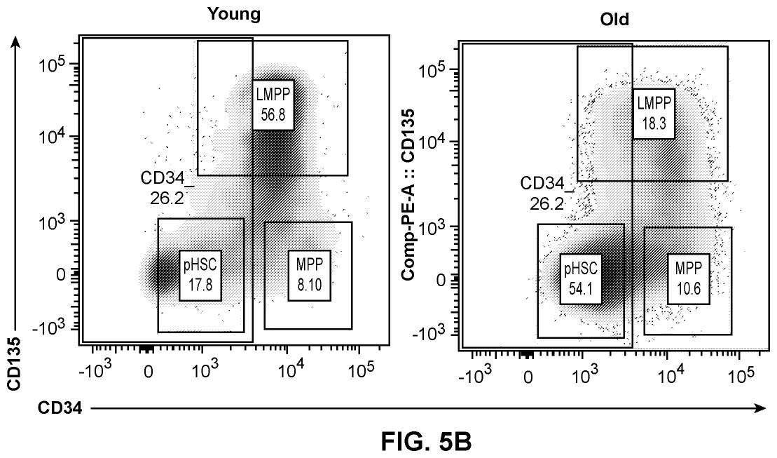

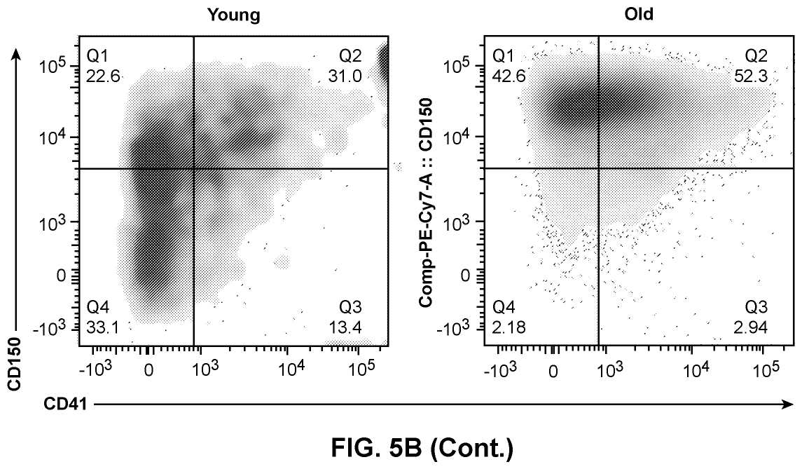

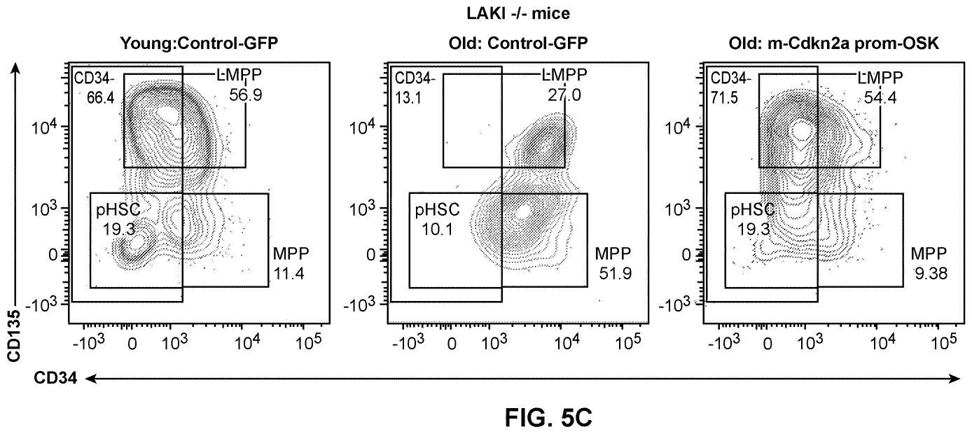

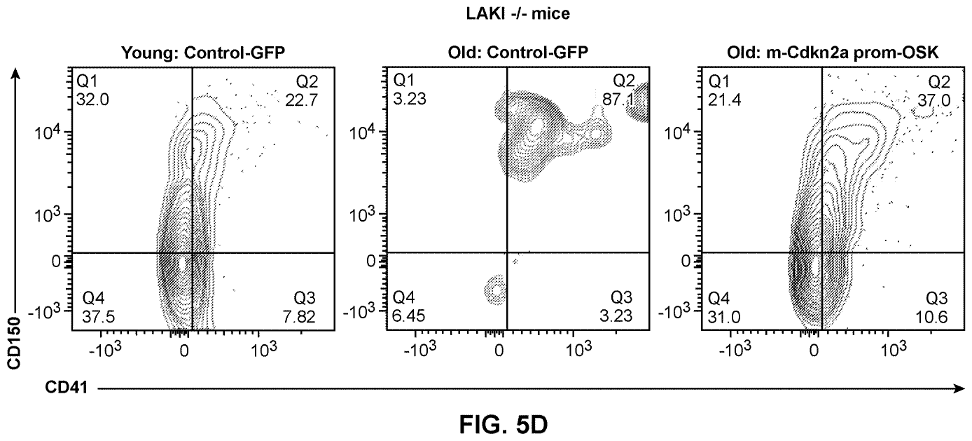

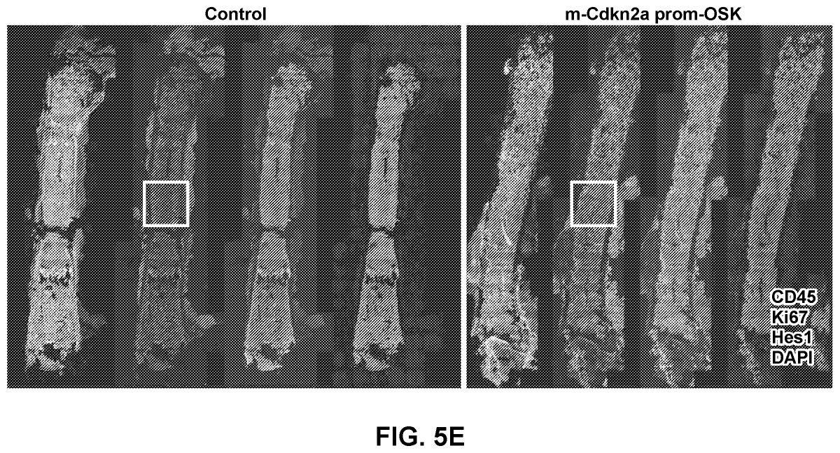

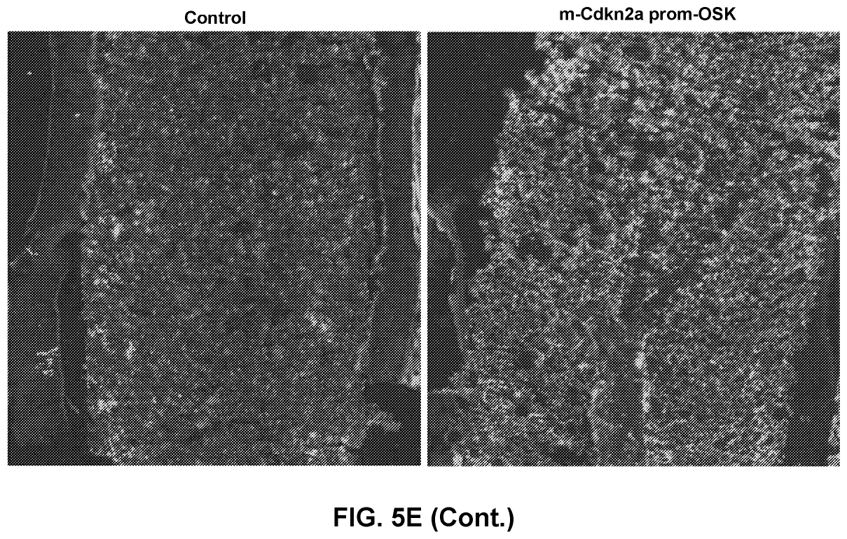

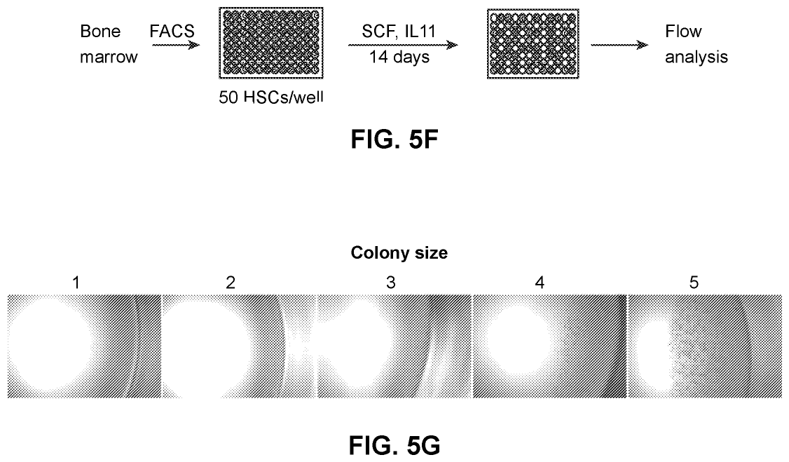

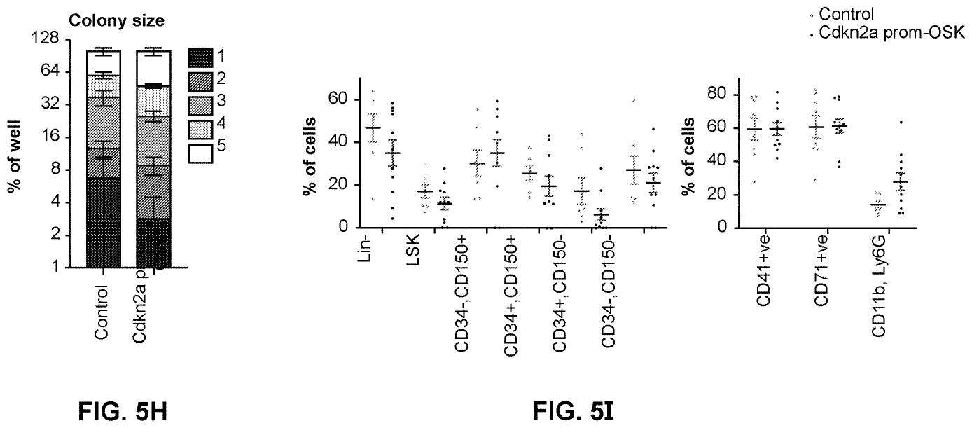

Upon treatment of old LAKI−/− mice with CDKN2A promoter-OSK AAV-DJ, a reversal of immune senescence in the bone marrow towards younger cellular states was observed in both the hematopoietic stem cell multipotent progenitor (HSC-MMP) and the hematopoietic stem cell (HSC) compartments (FIGS. 1E and 1F). Further, CDKN2A promoter-OSK AAV-DJ treatment reversed the accelerated depletion of HSC populations in LAKI−/− mice (FIG. 5A-5D). For example, increased expression of Ki67 in CDKN2A promoter-OSK AAV-DJ treated mice compared to constitutive promoter-GFP AAV-DJ controls indicated increased proliferation in the bone marrow compartment (FIG. 5E). Further, an increase in the percentage of CD34(−) CD150(+) cells in cultured bone marrow cells of CDKN2A promoter-OSK AAV-DJ treated LAKI−/− mice compared to CDKN2A promoter-GFP AAV-DJ controls indicated an increase in stem cells upon CDKN2A promoter-OSK AAV-DJ treatment (FIG. 5I). And a reversal of certain immune cell populations towards a younger state was observed in CDKN2A promoter-OSK AAV-DJ treated LAKI−/− mice (FIG. 5J).

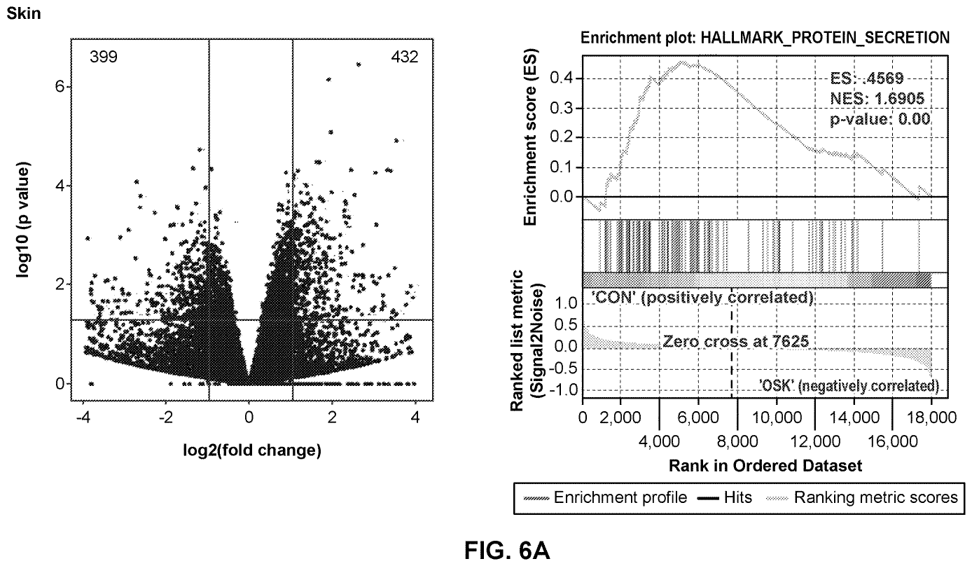

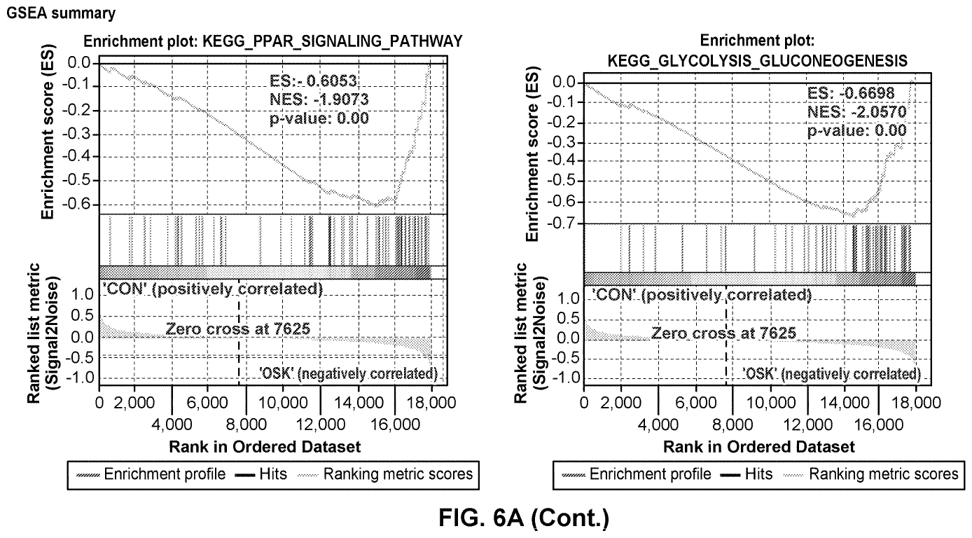

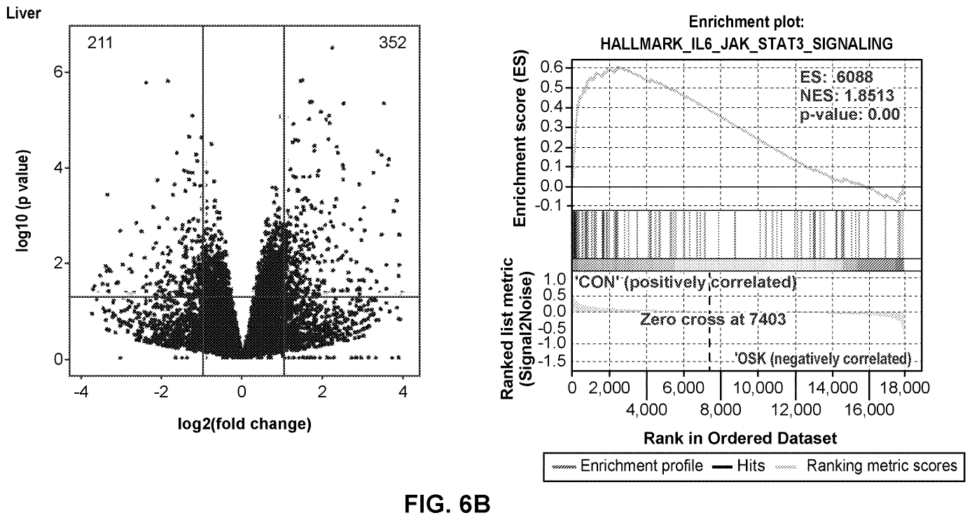

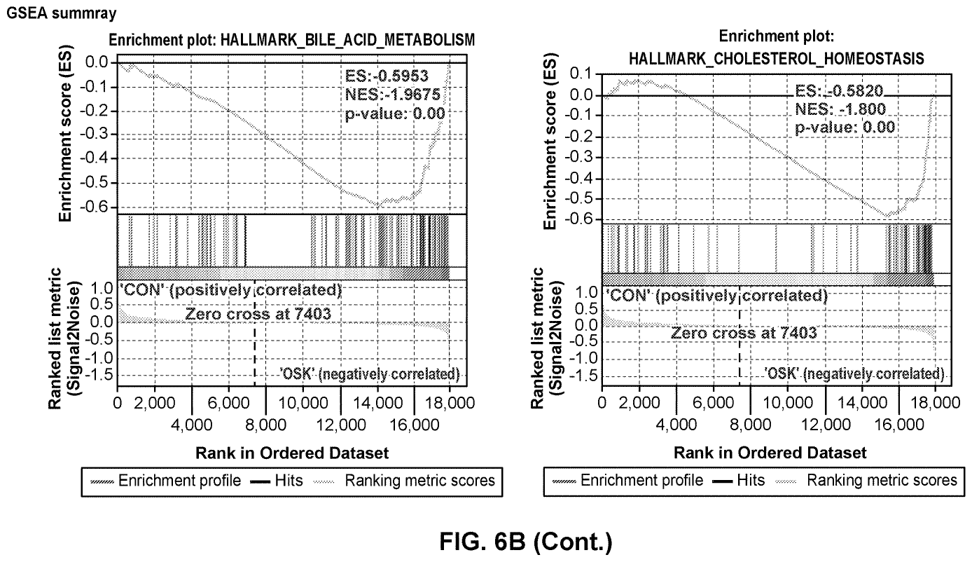

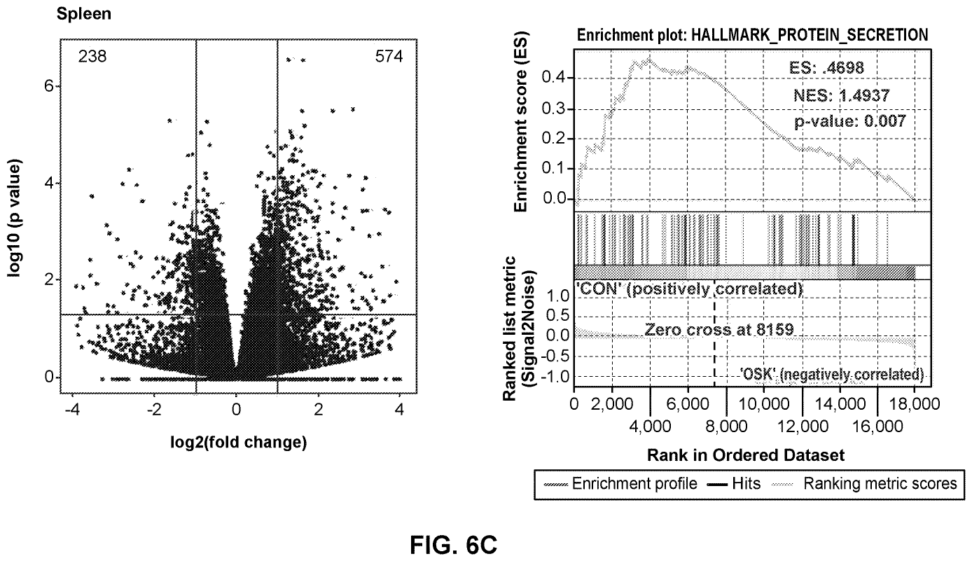

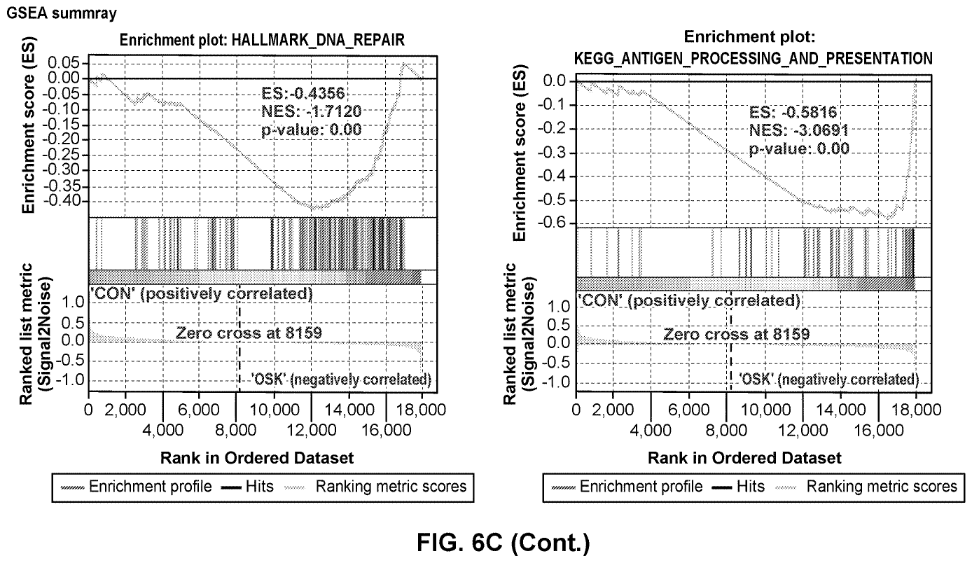

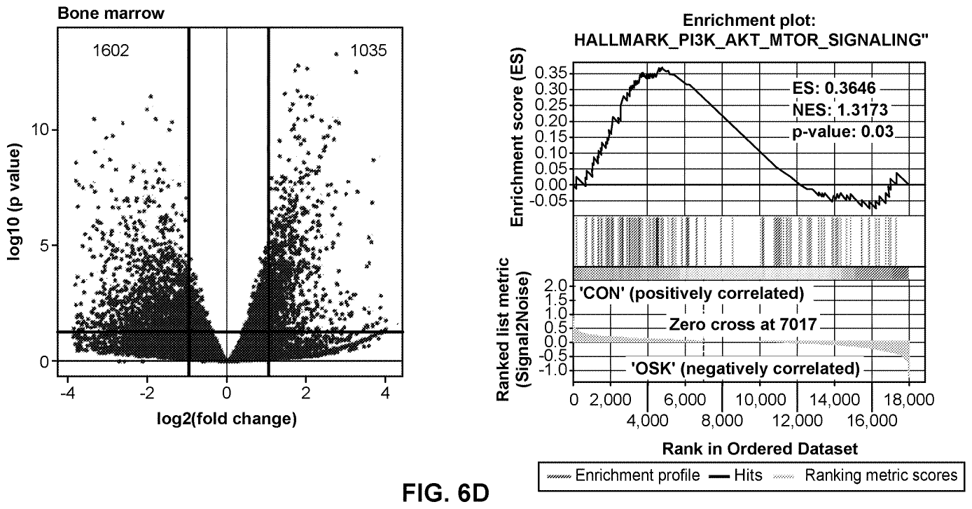

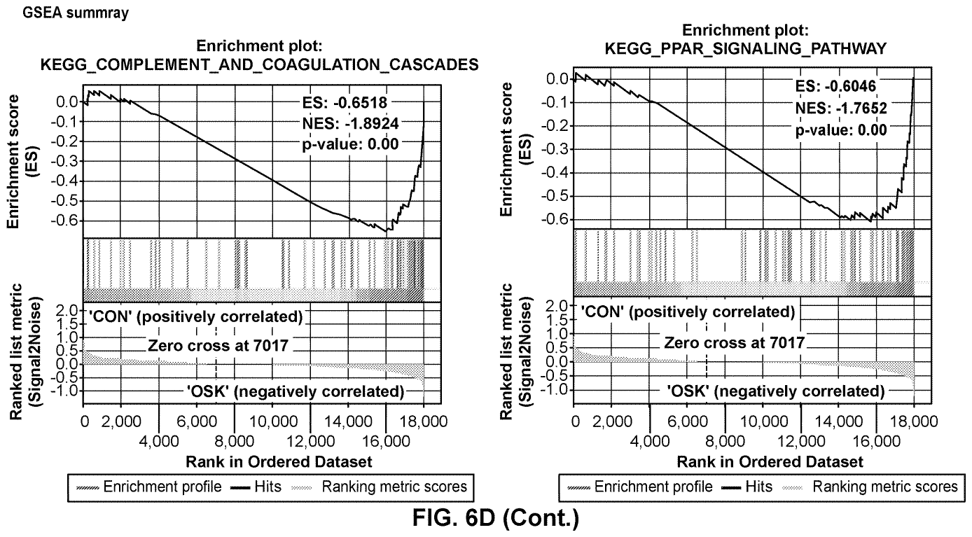

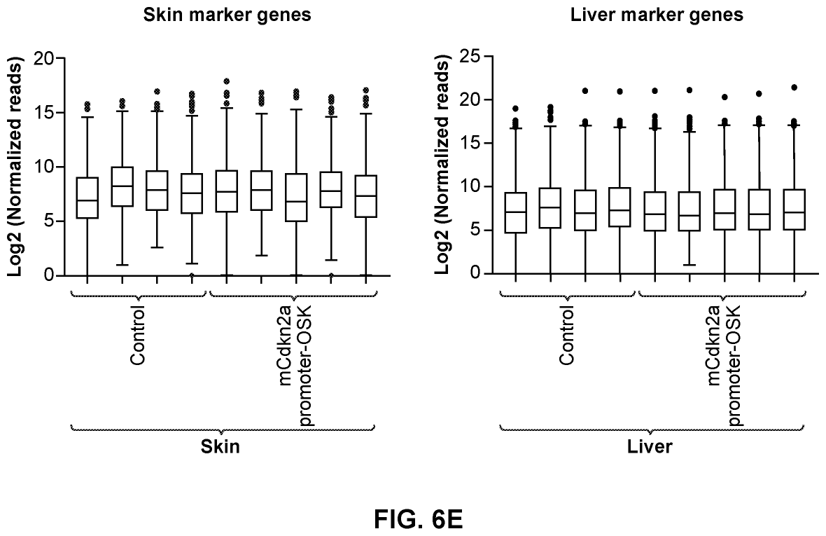

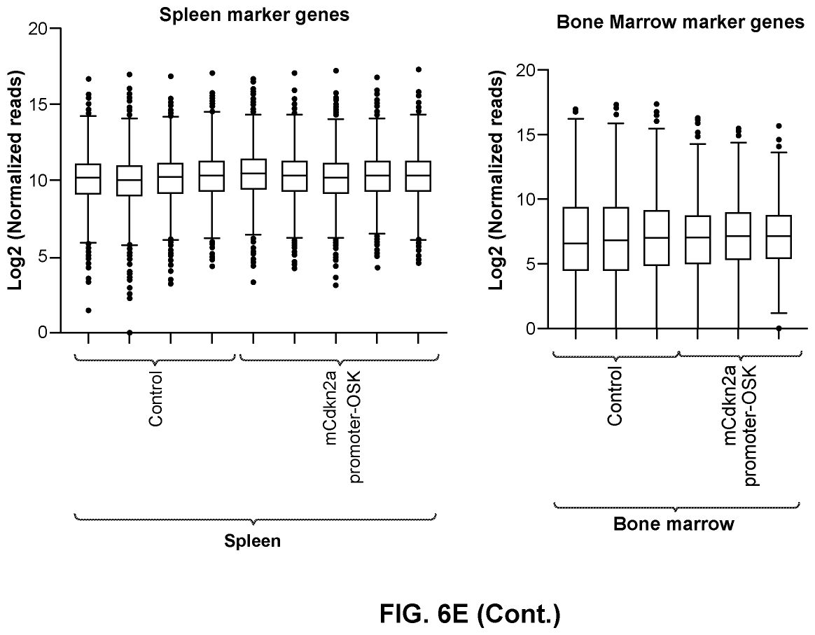

Bulk transcriptome analysis of skin, liver, spleen, and bone marrow showed distinct tissue-specific transcriptome alterations upon CDKN2A promoter-OSK AAV-DJ delivery in LAKI−/− mice (FIG. 1H, 6A-6D), while the overall organ-specific transcriptome was unchanged indicating that CDKN2A promoter-OSK did not change cell identity while repressing inflammation (FIG. 6E). Further, the immune compartment associated organs such as bone marrow and spleen showed more pronounced transcriptional changes when compared to other tested tissues (FIG. 1H, 6A-6D). For example, bone marrow and spleen transcriptomes showed a reduction in inflammatory signals and an activation of immune clearance pathways (FIGS. 1H, 6C, and 6D). Together, the results showed novel possibilities for therapeutic interventions, both in vivo and ex vivo, to restore bone marrow tissue function and thus increase health span.

### Example 5—Reversal of Senescence in Human Cells In Vitro

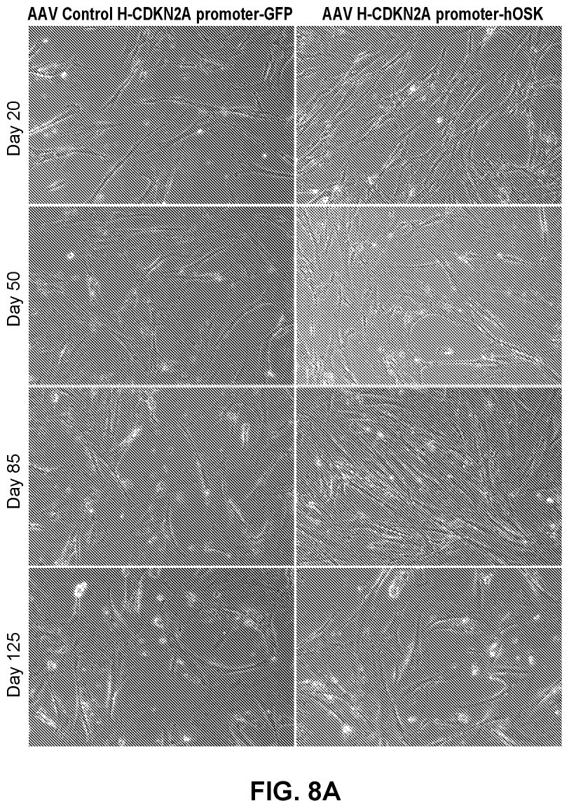

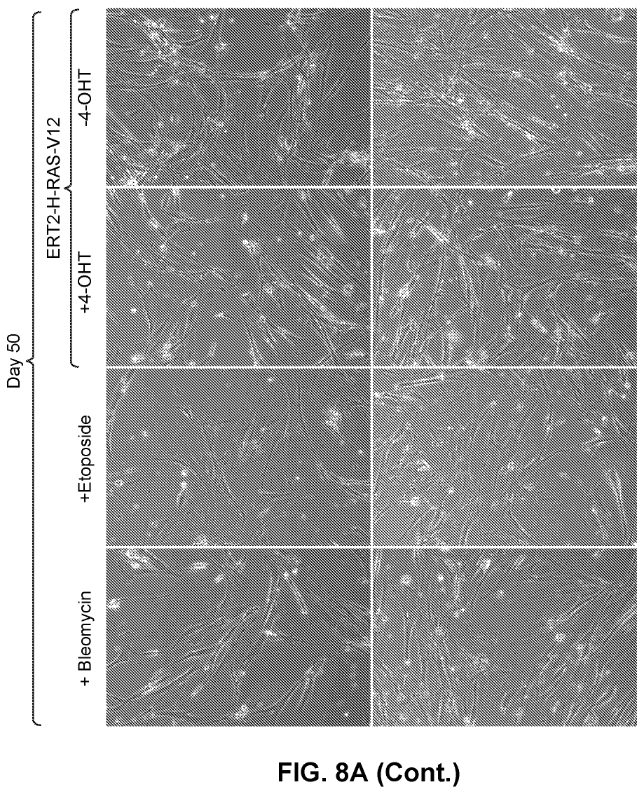

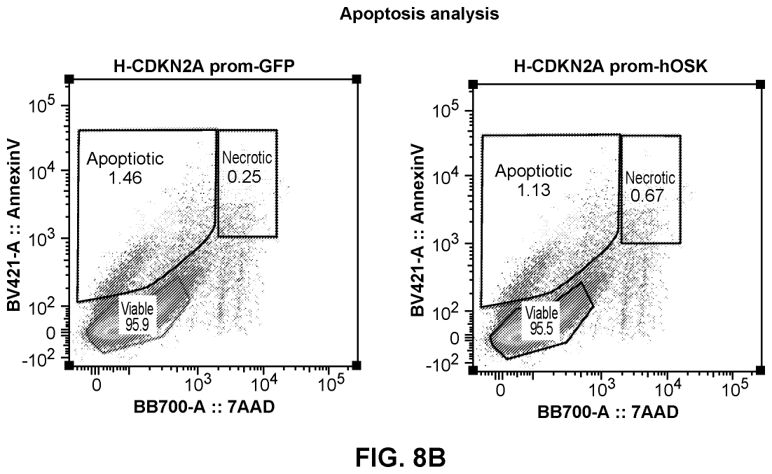

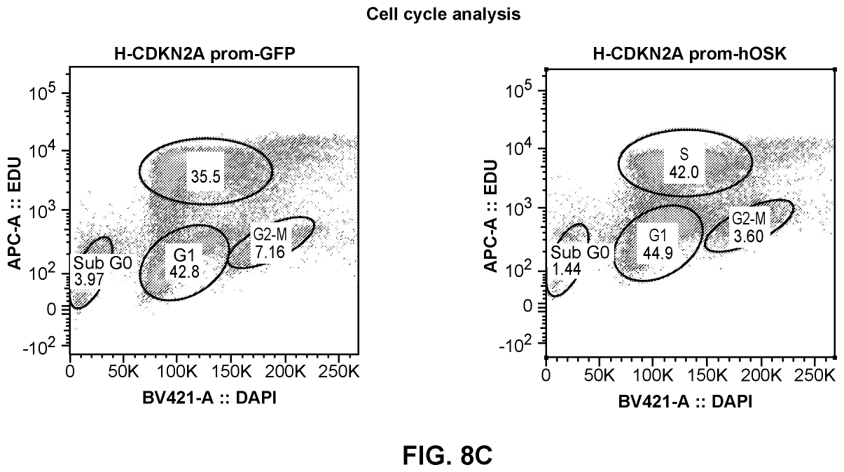

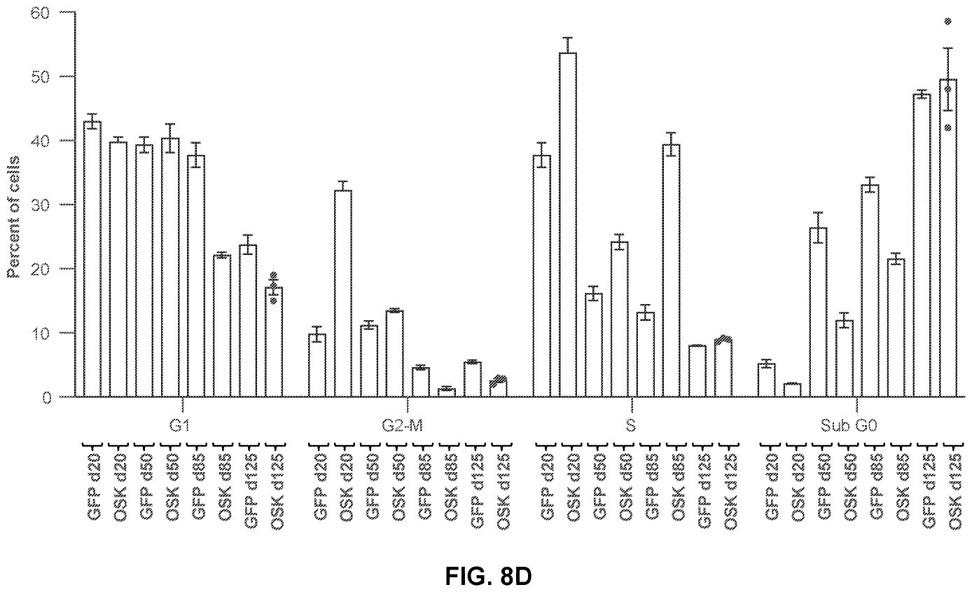

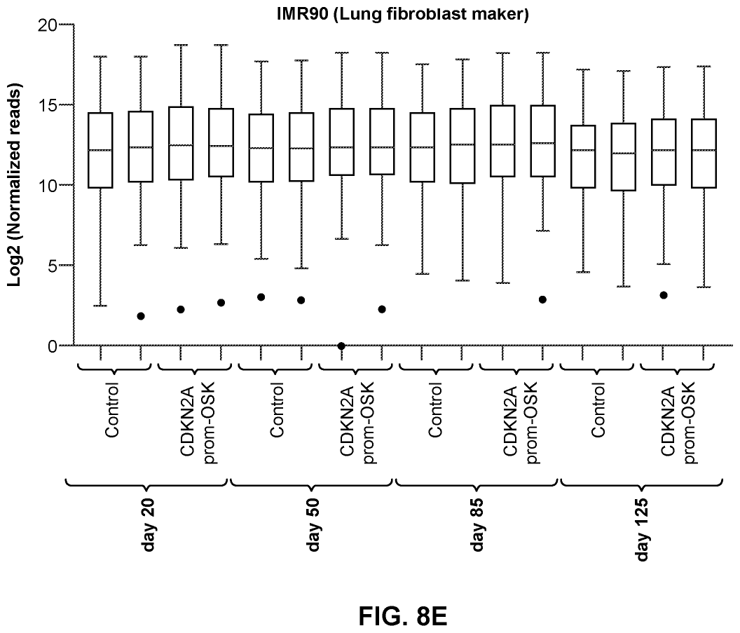

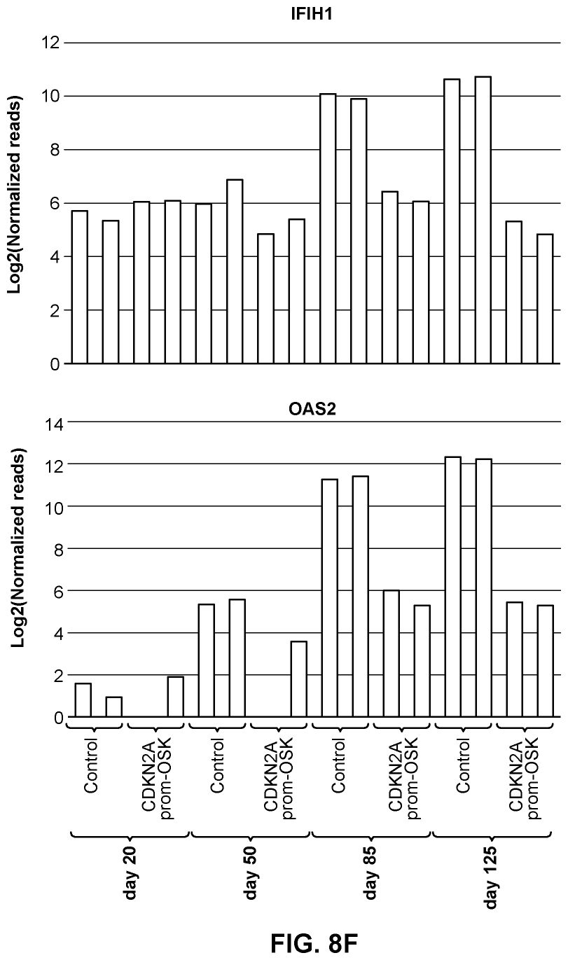

To further understand the fate of cells upon senescent cell-specific OSK expression, human primary lung fibroblast cells (IMR90) adopted to various senescence stages were used. During replicative senescence, human OSK (hOSK) expression under the control of the human CDKN2A promoter promoter resulted in transcriptional reversal towards a younger stage (FIGS. 7A, 7B, 8A, 9A and 9B). Interestingly, while hCDKN2A promoter-hOSK strongly and specifically reversed inflammatory responses during replicative senescence (FIGS. 7C, 8F, and 9C), CDKN2A promoter-hOSK did not have a substantial impact on cell cycle related genes in senescent cells (FIGS. 7D, 8C, 8D, and 9C) and increased cell cycling only in younger cells (FIG. 8D). Furthermore, the reversal of inflammatory responses in the aging transcriptome upon hCDKN2A promoter-hOSK treatment of human IMR90 cells occurred without altering cell identity (FIG. 8E).

Moreover, transcriptome analysis in senescent IMR90 cells transduced with hCDKN2A promoter-hOSK indicated a reduction in stress response and energy-consuming anabolic pathways, while activating a fraction of the extracellular matrix (ECM) compartment towards a healthier stage, e.g., resulting in increased expression of elastin and collagen 1A1 (COL1A1) upon hCDKN2A promoter-hOSK transduction (FIGS. 9D and 9E).

Similar transcriptional reversal indicating cellular rejuvenation was observed in DNA damage and oncogene (RAS) induced cellular senescence (FIGS. 7E, 8A, and 9F).

Notably, a reduction in p16 senescence marker expression was observed in CDKN2A promoter-OSK treated INR90 cells indicating a reduction in the senescent phenotype of the cells upon CDKN2A promoter-OSK treatment (FIG. 7A).

### Example 6—Long-Term Effects of Partial Reprogramming on Physiological Aging in Wild-Type Mice

To evaluate the effect of CDKN2A promoter-OSK during physiological aging, a single dose of CDKN2A promoter-OSK AAV-DJ or CDKN2A promoter-GFP AAV-DJ control was injected into 60 day old wild-type mice and 100% survival at 300 days was observed (FIG. 10A). Further, 18 months old wild-type mice were injected with a single dose of CDKN2A promoter-OSK AAV-DJ or CDKN2A promoter-GFP AAV-DJ control and maintenance of body weight, increased lifespan, and increased overall physical fitness was observed in CDKN2A promoter-OSK AAV-DJ injected mice compared to CDKN2A promoter-GFP AAV-DJ control mice (FIGS. 10B, 10C, and 11B).

Altogether, the results of the above Examples indicated activation of repair and rejuvenation pathways in p16 expressing cells following cellular partial reprogramming. No incidence of tumors was found upon long-term CDKN2A promoter-OSK expression in young mice, and a similar frequency of tumors was observed upon long-term CDKN2A promoter-OSK expression in old wild-type mice compared to controls (FIG. 11A).

The results described herein indicate that partial cellular reprogramming of senescent cells can have a broad organismal rejuvenation effect without the risks of tumor induction or organ failure due to loss of cell number and identity.

The sequences described herein are shown in Table 3.

TABLE 3

| SEQ ID | |
|---|---|
| NO | |
| 1 | GCGGGCCACATTTCCAGGTTCCCGGAGCTGGGCCTAGTCCCGAATCCT |
| Human | CTGGCACACACCCACCCACTCAGGCCGCGGGTCCAGCCCGCGAGGTTT |
| CDKN2A | AGGACGGATCCAGGCAGACCGCAGGCTCCGGGTCGGGGCACCGGGTC |
| promoter | AGCGCGCCGGCCTGAAGGCGGCGTCCTGGGCTCGACTTCCCGCGCGCG |
| | GAGAGCCGGCGAGCCCGCGTCCGAGTTCCTGGACGAGAGCCGAGCCT |
| | CGCTTAGACCGCGCTCAGGACCCGGCTCCTCCGCATTCTCCGGCTGCCC |
| | CTGTGTCCTCGACTCACCCCTCCTTTCTGCCGCTCCTTCCTTTCCTTGCC |
| | CTGCTTTTACTGTTCCCAAACAGGACCGCTTTTCCTGTCTCCCAGCTGG |
| | AAAGGAGGAAGGGAGAGAGTCCAGAAAGGATCGGTGATGTGGAAGA |
| | AAAGGGGAGGAGGGGACATGGAGGGGGAGACCGGAGAGAGAACGTA |
| | CGCCGAGGAGTCAGGCGGCGGGATCAAGGGGAGTCGGGGTGTCTGGG |
| | CGCGGGGCAGAGCGTGGAGGCGGCAGCGGCCAACGGTCGCCAAGACA |
| | ACCATTCTACGCGAGGACGCGGCGACAGGAGGGGAGCGGCCAGCAGG |
| | GGAGGGGAGCGCGGGGGAAGAGGAAAGAGGAAGAAGCGCTCAGATG |
| | CTCCGCGGCTGTCGTGAAGGTTAAAACCGAAAATAAAAATGGGCTAG |
| | ACACAAAGGACTCGGTGCTTGTCCCAGCCAGGCGCCCTCGGCGACGCG |
| | GGCAGCTGGGAGGGGAATGGGCGCCCGGACCCAGCTGGGACCCCCGG |
| | GTGCGACTCCACCTACCTAGTCCGGCGCCAGGCCGGGTCGACAGCTCC |
| | GGCAGCGCCAGCGCCGCGCCGTGTCCAGATGTCGCGTCAGAGGCGTGC |
| | AGCGGTTTAGTTTAATTTCGCTTGTTTTCCAAATCTAGAAGAGGAGCGG |
| | AGCGGCTTTTAGTTCAAAACTGACATTCAGCCTCCTGATTGGCGGATA |
| | GAGCAATGAGATGACCTCGCTTTCCTTTCTTCCTTTTTCATTTTTAAATA |
| | ATCTAGTTTGAAGAATGGAAGACTTTCGACGAGGGGAGCCAGGAATA |
| | AAATAAGGGGAATAGGGGAGCGGGGACGCGAGCAGCACCAGAATCCG |
| | CGGGAGCGCGGCTGTTCCTGGTAGGGCCGTGTCAGGTGACGGATGTAG |
| | CTAGGGGGCGAGCTGCCTGGAGTTGCGTTCCAGGCGTCCGGCCCCTGG |
| | GCCGTCACCGCGGGGCGCCCGCGCTGAGGGTGGGAAGATGGTGGTGG |
| | GGGTGGGGGCGCACACAGGGCGGGAAAGTGGCGGTAGGCGGGAGGG |
| | AGAGGAACGCGGGCCCTGAGCCGCCCGCGCGCGCGCCTCCCTACGGG |
| | CGCCTCCGGCAGCCCTTCCCGCGTGCGCAGGGCTCAGAGCCGTTCCGA |
| | GATCTTGGAGGTCCGGGTGGGAGTGGGGGTGGGGTGGGGGTGGGGGT |
| | GAAGGTGGGGGGCGGGCGCGCTCAGGGAAGGCGGGTGCGCGCCTGCG |
| | GGGCGGAGATGGGCAGGGGGCGGTGCGTGGGTCCCAGTCTGCAGTTA |
| | AGGGGGCAGGAGTGGCGCTGCTCACCTCTGGTGCCAAAGGGCGGCGC |
| | AGCGGCTGCCGAGCTCGGCCCTGGAGGCGGCGAGAACATGGTG |
| 2 | TTTTCCTGTTTCGCGGGGGCCGGGGTGGAGGCGGAGAAGGAGGCGAG |
| Mouse | GAGGCAGCGGAGAAGGGGGCAGCCGGGAGGGGGGAGGGGACCGGAG |
| CDKN2A | AAGGAGGCGACGCGGGGGGGGAAAGAGGAAGAGCCGGGGATGCTC |
| promoter | GCGCTTAAAACCGAAAATAAACAACGTCCTCGGGCTGCGGCCTCGTGT |
| | CTGCCCGACCGCGCGGGGGCCTGGCCGGGACACACTTACCGGTCCGCC |
| | GCGGCGTGCAGGCCCTGGTCCCGGGTACACGCAGCGGCTCTTCTCTGC |
| | GCGCTGTTCCCAACCGGAGGAGCGCAGCGTACCGCTAAGGGTTCAAAA |
| | CGCCCGCGCGCGTCCTGATTGGCGGGCCGGCCCATGGCTGGCCTCGCC |
| | GATCTTCCTATTTTCTTCTTTTTATAGATGGACTCGGAGCAAGGGAAAC |
| | CTCGCCGAGGGGCGTCAGGCATAGACGAAAGTTTCGGGACCCGAAAG |
| | TTAACCGGAGCGAAAGCCGATCGGCGCAGGAGACGTTGCCGGCCGGC |
| | TGCGAAAGGGCGCAGCTACTGCTAGGGGGCGGGCGGTGCGCACGCGT |
| | CCCGCGGCGCTGGCTGTCACCGCGATGGGTGGCGAGCGAAGCGAGCG |
| | GGATCCGGAGCGTGCCCTGCGCGGGAGGCAGCGGGACCCCGGATGCG |
| | GCAGGGCCCGCGCGCGCCTCCCCCTGGGCGCCTCTGGGAAGCTTTCCC |
| | GCGCGACTGGGGACCGCGCGCCTTGGGACTCGGGGGCGCGCCTGAGG |
| | GCGGAGATGGGCGTGGAGCAAAGATGGGCCGGGGGCGGCGCGTGGGT |
| | CTCGAGGTGCCTCAACGCCGAAGGGGCTGGGGGCGGCGCTTCTCACCT |
| | CGCTTGTCACAGTGAGGCCGCCGCTGAGGGAGTACAGCAGCGGGAGC |
| 3 | ATGGCGGGACACCTGGCTTCGGATTTCGCCTTCTCGCCCCCTCCAGGTG |
| Human | GTGGAGGTGATGGGCCAGGGGGGCCGGAGCCGGGCTGGGTTGATCCT |
| Oct4 | CGGACCTGGCTAAGCTTCCAAGGCCCTCCTGGAGGGCCAGGAATCGGG |
| nucleic | CCGGGGGTTGGGCCAGGCTCTGAGGTGTGGGGGATTCCCCCATGCCCC |
| acid | CCGCCGTATGAGTTCTGTGGGGGGATGGCGTACTGTGGGCCCCAGGTT |
| sequence | GGAGTGGGGCTAGTGCCCCAAGGCGGCTTGGAGACCTCTCAGCCTGAG |
| | GGCGAAGCAGGAGTCGGGGTGGAGAGCAACTCCGATGGGGCCTCCCC |
| | GGAGCCCTGCACCGTCACCCCTGGTGCCGTGAAGCTGGAGAAGGAGA |
| | AGCTGGAGCAAAACCCGGAGGAGTCCCAGGACATCAAAGCTCTGCAG |
| | AAAGAACTCGAGCAATTTGCCAAGCTCCTGAAGCAGAAGAGGATCAC |
| | CCTGGGATATACACAGGCCGATGTGGGGCTCACCCTGGGGGTTCTATT |
| | TGGGAAGGTATTCAGCCAAACGACCATCTGCCGCTTTGAGGCTCTGCA |
| | GCTTAGCTTCAAGAACATGTGTAAGCTGCGGCCCTTGCTGCAGAAGTG |
| | GGTGGAGGAAGCTGACAACAATGAAAATCTTCAGGAGATATGCAAAG |
| | CAGAAACCCTCGTGCAGGCCCGAAAGAGAAAGCGAACCAGTATCGAG |
| | AACCGAGTGAGAGGCAACCTGGAGAATTTGTTCCTGCAGTGCCCGAAA |
| | CCCACACTGCAGCAGATCAGCCACATCGCCCAGCAGCTTGGGCTCGAG |
| | AAGGATGTGGTCCGAGTGTGGTTCTGTAACCGGCGCCAGAAGGGCAA |
| | GCGATCAAGCAGCGACTATGCACAACGAGAGGATTTTGAGGCTGCTGG |
| | GTCTCCTTTCTCAGGGGGACCAGTGTCCTTTCCTCTGGCCCCAGGGCCC |
| | CATTTTGGTACCCCAGGCTATGGGAGCCCTCACTTCACTGCACTGTACT |
| | CCTCGGTCCCTTTCCCTGAGGGGGAAGCCTTTCCCCCTGTCTCCGTCAC |
| | CACTCTGGGCTCTCCCATGCATTCAAAC |
| 4 | ATGTACAACATGATGGAGACGGAGCTGAAGCCGCCGGGCCCGCAGCA |
| Human | AACTTCGGGGGGCGGCGGCGGCAACTCCACCGCGGCGGCGGCCGGCG |
| Sox2 | GCAACCAGAAAAACAGCCCGGACCGCGTCAAGCGGCCCATGAATGCC |
| nucleic | TTCATGGTGTGGTCCCGCGGGCAGCGGCGCAAGATGGCCCAGGAGAA |
| acid | CCCCAAGATGCACAACTCGGAGATCAGCAAGCGCCTGGGCGCCGAGT |
| sequence | GGAAACTTTTGTCGGAGACGGAGAAGCGGCCGTTCATCGACGAGGCTA |
| | AGCGGCTGCGAGCGCTGCACATGAAGGAGCACCCGGATTATAAATAC |
| | CGGCCCCGGCGGAAAACCAAGACGCTCATGAAGAAGGATAAGTACAC |
| | GCTGCCCGGCGGGCTGCTGGCCCCCGGCGGCAATAGCATGGCGAGCG |
| | GGGTCGGGGTGGGCGCCGGCCTGGGCGCGGGCGTGAACCAGCGCATG |
| | GACAGTTACGCGCACATGAACGGCTGGAGCAACGGCAGCTACAGCAT |
| | GATGCAGGACCAGCTGGGCTACCCGCAGCACCCGGGCCTCAATGCGCA |
| | CGGCGCAGCGCAGATGCAGCCCATGCACCGCTACGACGTGAGCGCCCT |
| | GCAGTACAACTCCATGACCAGCTCGCAGACCTACATGAACGGCTCGCC |
| | CACCTACAGCATGTCCTACTCGCAGCAGGGCACCCCTGGCATGGCTCT |
| | TGGCTCCATGGGTTCGGTGGTCAAGTCCGAGGCCAGCTCCAGCCCCCC |
| | TGTGGTTACCTCTTCCTCCCACTCCAGGGCGCCCTGCCAGGCCGGGGA |
| | CCTCCGGGACATGATCAGCATGTATCTCCCCGGCGCCGAGGTGCCGGA |
| | ACCCGCCGCCCCCAGCAGACTTCACATGTCCCAGCACTACCAGAGCGG |
| | CCCGGTGCCCGGCACGGCCATTAACGGCACACTGCCCCTCTCACACAT |
| | G |
| 5 | ATGGCTGTCAGCGACGCGCTGCTCCCATCTTTCTCCACGTTCGCGTCTG |
| Human | GCCCGGCGGGAAGGGAGAAGACACTGCGTCAAGCAGGTGCCCCGAAT |
| Klf4 | AACCGCTGGCGGGAGGAGCTCTCCCACATGAAGCGACTTCCCCCAGTG |
| nucleic | CTTCCCGGCCGCCCCTATGACCTGGCGGCGGCGACCGTGGCCACAGAC |
| acid | CTGGAGAGCGGCGGAGCCGGTGCGGCTTGCGGCGGTAGCAACCTGGC |
| sequence | GCCCCTACCTCGGAGAGAGACCGAGGAGTTCAACGATCTCCTGGACCT |
| | GGACTTTATTCTCTCCAATTCGCTGACCCATCCTCCGGAGTCAGTGGCC |
| | GCCACCGTGTCCTCGTCAGCGTCAGCCTCCTCTTCGTCGTCGCCGTCGA |
| | GCAGCGGCCCTGCCAGCGCGCCCTCCACCTGCAGCTTCACCTATCCGA |
| | TCCGGGCCGGGAACGACCCGGGCGTGGCGCCGGGCGGCACGGGCGGA |
| | GGCCTCCTCTATGGCAGGGAGTCCGCTCCCCCTCCGACGGCTCCCTTCA |
| | ACCTGGCGGACATCAACGACGTGAGCCCCTCGGGCGGCTTCGTGGCCG |
| | AGCTCCTGCGGCCAGAATTGGACCCGGTGTACATTCCGCCGCAGCAGC |
| | CGCAGCCGCCAGGTGGCGGGCTGATGGGCAAGTTCGTGCTGAAGGCGT |
| | CGCTGAGCGCCCCTGGCAGCGAGTACGGCAGCCCGTCGGTCATCAGCG |
| | TCAGCAAAGGCAGCCCTGACGGCAGCCACCCGGTGGTGGTGGCGCCCT |
| | ACAACGGCGGGCCGCCGCGCACGTGCCCCAAGATCAAGCAGGAGGCG |
| | GTCTCTTCGTGCACCCACTTGGGCGCTGGACCCCCTCTCAGCAATGGCC |
| | ACCGGCCGGCTGCACACGACTTCCCCCTGGGGCGGCAGCTCCCCAGCA |
| | GGACTACCCCGACCCTGGGTCTTGAGGAAGTGCTGAGCAGCAGGGACT |
| | GTCACCCTGCCCTGCCGCTTCCTCCCGGCTTCCATCCCCACCCGGGGCC |
| | CAATTACCCATCCTTCCTGCCCGATCAGATGCAGCCGCAAGTCCCGCC |
| | GCTCCATTACCAAGAGCTCATGCCACCCGGTTCCTGCATGCCAGAGGA |
| | GCCCAAGCCAAAGAGGGGAAGACGATCGTGGCCCCGGAAAAGGACCG |
| | CCACCCACACTTGTGATTACGCGGGCTGCGGCAAAACCTACACAAAGA |
| | GTTCCCATCTCAAGGCACACCTGCGAACCCACACAGGTGAGAAACCTT |
| | ACCACTGTGACTGGGACGGCTGTGGATGGAAATTCGCCCGCTCAGATG |
| | AACTGACCAGGCACTACCGTAAACACACGGGGCACCGCCCGTTCCAGT |
| | GCCAAAAATGCGACCGAGCATTTTCCAGGTCGGACCACCTCGCCTTAC |
| | ACATGAAGAGGCATTTTTAA |
| 6 | RXKRR |
| furin | |
| cleavage | |
| site | |
| consensus | |
| amino | |
| acid | |
| sequence | |
| 7 | ATGTTCCAGGCGGCCGAGCGTCCCCAGGAGTGGGCCATGGAGGGCCCC |
| dominant | CGCGACGGGCTGAAGAAGGAGCGGCTACTGGACGACCGCCACGACGC |
| negative | CGGCCTGGACGCCATGAAAGACGAGGAGTACGAGCAGATGGTCAAGG |
| NFκBIA | AGCTGCAGGAGATCCGCCTCGAGCCGCAGGAGGTGCCGCGCGGCTCG |
| nucleic | GAGCCCTGGAAGCAGCAGCTCACCGAGGACGGGGACTCGTTCCTGCAC |
| acid | TTGGCCATCATCCATGAAGAAAAGGCACTGACCATGGAAGTGATCCGC |
| sequence | CAGGTGAAGGGAGACCTGGCCTTCCTCAACTTCCAGAACAACCTGCAG |
| | CAGACTCCACTCCACTTGGCTGTGATCACCAACCAGCCAGAAATTGCT |
| | GAGGCACTTCTGGGAGCTGGCTGTGATCCTGAGCTCCGAGACTTTCGA |
| | GGAAATACCCCCCTACACCTTGCCTGTGAGCAGGGCTGCCTGGCCAGC |
| | GTGGGAGTCCTGACTCAGTCCTGCACCACCCCGCACCTCCACTCCATCC |
| | TGAAGGCTACCAACTACAATGGCCACACGTGTCTACACTTAGCCTCTA |
| | TCCATGGCTACCTGGGCATCGTGGAGCTTTTGGTGTCCTTGGGTGCTGA |
| | TGTCAATGCTCAGGAGCCCTGTAATGGCCGGACTGCCCTTCACCTCGC |
| | AGTGGACCTGCAAAATCCTGACCTGGTGTCACTCCTGTTGAAGTGTGG |
| | GGCTGATGTCAACAGAGTTACCTACCAGGGCTATTCTCCCTACCAGCT |
| | CACCTGGGGCCGCCCAAGCACCCGGATACAGCAGCAGCTGGGCCAGC |
| | TGACACTAGAAAACCTTCAGATGCTGCCAGAGAGTGAGGATGAGGAG |
| | AGCTATGACACAGAGTCAGAGTTCACGGAGTTCACAGAGGACGAGCT |
| | GCCCTATGATGACTGTGTGTTTGGAGGCCAGCGTCTGACGTTA |
| 8 | CCCCTCTCCCTCCCCCCCCCCTAACGTTACTGGCCGAAGCCGCTTGGAA |
| IRES | TAAGGCCGGTGTGCGTTTGTCTATATGTTATTTTCCACCATATTGCCGT |
| nucleic | CTTTTGGCAATGTGAGGGCCCGGAAACCTGGCCCTGTCTTCTTGACGA |
| acid | GCATTCCTAGGGGTCTTTCCCCTCTCGCCAAAGGAATGCAAGGTCTGTT |
| sequence | GAATGTCGTGAAGGAAGCAGTTCCTCTGGAAGCTTCTTGAAGACAAAC |
| | AACGTCTGTAGCGACCCTTTGCAGGCAGCGGAACCCCCCACCTGGCGA |
| | CAGGTGCCTCTGCGGCCAAAAGCCACGTGTATAAGATACACCTGCAAA |
| | GGCGGCACAACCCCAGTGCCACGTTGTGAGTTGGATAGTTGTGGAAAG |
| | AGTCAAATGGCTCTCCTCAAGCGTATTCAACAAGGGGCTGAAGGATGC |
| | CCAGAAGGTACCCCATTGTATGGGATCTGATCTGGGGCCTCGGTGCAC |
| | ATGCTTTACATGTGTTTAGTCGAGGTTAAAAAAACGTCTAGGCCCCCC |
| | GAACCACGGGGACGTGGTTTTCCTTTGAAAAACACGA |
| 9 | CATTAATGAATCGGCCAACGCGCGGGGAGAGGCGGTTTGCGTATTGGG |
| AAV | CGCTCTTCCGCTTCCTCGCTCACTGACTCGCTGCGCTCGGTCGTTCGGC |
| CAG- | TGCGGCGAGCGGTATCAGCTCACTCAAAGGCGGTAATACGGTTATCCA |
| CBRLuc- | CAGAATCAGGGGATAACGCAGGAAAGAACATGTGAGCAAAAGGCCAG |
| GFP | CAAAAGGCCAGGAACCGTAAAAAGGCCGCGTTGCTGGCGTTTTTCCAT |
| nucleic | AGGCTCCGCCCCCCTGACGAGCATCACAAAAATCGACGCTCAAGTCAG |
| acid | AGGTGGCGAAACCCGACAGGACTATAAAGATACCAGGCGTTTCCCCCT |
| sequence | GGAAGCTCCCTCGTGCGCTCTCCTGTTCCGACCCTGCCGCTTACCGGAT |
| | ACCTGTCCGCCTTTCTCCCTTCGGGAAGCGTGGCGCTTTCTCATAGCTC |
| | ACGCTGTAGGTATCTCAGTTCGGTGTAGGTCGTTCGCTCCAAGCTGGG |
| | CTGTGTGCACGAACCCCCCGTTCAGCCCGACCGCTGCGCCTTATCCGGT |
| | AACTATCGTCTTGAGTCCAACCCGGTAAGACACGACTTATCGCCACTG |
| | GCAGCAGCCACTGGTAACAGGATTAGCAGAGCGAGGTATGTAGGCGG |
| | TGCTACAGAGTTCTTGAAGTGGTGGCCTAACTACGGCTACACTAGAAG |
| | AACAGTATTTGGTATCTGCGCTCTGCTGAAGCCAGTTACCTTCGGAAA |
| | AAGAGTTGGTAGCTCTTGATCCGGCAAACAAACCACCGCTGGTAGCGG |
| | TGGTTTTTTTGTTTGCAAGCAGCAGATTACGCGCAGAAAAAAAGGATC |
| | TCAAGAAGATCCTTTGATCTTTTCTACGGGGTCTGACGCTCAGTGGAAC |
| | GAAAACTCACGTTAAGGGATTTTGGTCATGAGATTATCAAAAAGGATC |
| | TTCACCTAGATCCTTTTAAATTAAAAATGAAGTTTTAAATCAATCTAAA |
| | GTATATATGAGTAAACTTGGTCTGACAGTTACCAATGCTTAATCAGTG |
| | AGGCACCTATCTCAGCGATCTGTCTATTTCGTTCATCCATAGTTGCCTG |
| | ACTCCCCGTCGTGTAGATAACTACGATACGGGAGGGCTTACCATCTGG |
| | CCCCAGTGCTGCAATGATACCGCGAGACCCACGCTCACCGGCTCCAGA |
| | TTTATCAGCAATAAACCAGCCAGCCGGAAGGGCCGAGCGCAGAAGTG |
| | GTCCTGCAACTTTATCCGCCTCCATCCAGTCTATTAATTGTTGCCGGGA |
| | AGCTAGAGTAAGTAGTTCGCCAGTTAATAGTTTGCGCAACGTTGTTGC |
| | CATTGCTACAGGCATCGTGGTGTCACGCTCGTCGTTTGGTATGGCTTCA |
| | TTCAGCTCCGGTTCCCAACGATCAAGGCGAGTTACATGATCCCCCATG |
| | TTGTGCAAAAAAGCGGTTAGCTCCTTCGGTCCTCCGATCGTTGTCAGA |
| | AGTAAGTTGGCCGCAGTGTTATCACTCATGGTTATGGCAGCACTGCAT |
| | AATTCTCTTACTGTCATGCCATCCGTAAGATGCTTTTCTGTGACTGGTG |
| | AGTACTCAACCAAGTCATTCTGAGAATAGTGTATGCGGCGACCGAGTT |
| | GCTCTTGCCCGGCGTCAATACGGGATAATACCGCGCCACATAGCAGAA |
| | CTTTAAAAGTGCTCATCATTGGAAAACGTTCTTCGGGGCGAAAACTCT |
| | CAAGGATCTTACCGCTGTTGAGATCCAGTTCGATGTAACCCACTCGTG |
| | CACCCAACTGATCTTCAGCATCTTTTACTTTCACCAGCGTTTCTGGGTG |
| | AGCAAAAACAGGAAGGCAAAATGCCGCAAAAAAGGGAATAAGGGCG |
| | ACACGGAAATGTTGAATACTCATACTCTTCCTTTTTCAATATTATTGAA |
| | GCATTTATCAGGGTTATTGTCTCATGAGCGGATACATATTTGAATGTAT |
| | TTAGAAAAATAAACAAATAGGGGTTCCGCGCACATTTCCCCGAAAAGT |
| | GCCACCTAAATTGTAAGCGTTAATATTTTGTTAAAATTCGCGTTAAATT |
| | TTTGTTAAATCAGCTCATTTTTTAACCAATAGGCCGAAATCGGCAAAAT |
| | CCCTTATAAATCAAAAGAATAGACCGAGATAGGGTTGAGTGTTGTTCC |
| | AGTTTGGAACAAGAGTCCACTATTAAAGAACGTGGACTCCAACGTCAA |
| | AGGGCGAAAAACCGTCTATCAGGGCGATGGCCCACTACGTGAACCATC |
| | ACCCTAATCAAGTTTTTTGGGGTCGAGGTGCCGTAAAGCACTAAATCG |
| | GAACCCTAAAGGGAGCCCCCGATTTAGAGCTTGACGGGGAAAGCCGG |
| | CGAACGTGGCGAGAAAGGAAGGGAAGAAAGCGAAAGGAGCGGGCGC |
| | TAGGGCGCTGGCAAGTGTAGCGGTCACGCTGCGCGTAACCACCACACC |
| | CGCCGCGCTTAATGCGCCGCTACAGGGCGCGTCCCATTCGCCATTCAG |
| | GCTGCGCAACTGTTGGGAAGGGCGATCGGTGCGGGCCTCTTCGCTATT |
| | ACGCCAGCTGCGCGCTCGCTCGCTCACTGAGGCCGCCCGGGCAAAGCC |
| | CGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTT |
| | AATGATTAACCCGCCATGCTACTTATCTACGTAGCCATGCTCTAGGAA |
| | GAGTACCATTGACGTCAATAATGACGTATGTTCCCATAGTAACGCCAA |
| | TAGGGACTTTCCATTGACGTCAATGGGTGGAGTATTTACGGTAAACTG |
| | CCCACTTGGCAGTACATCAAGTGTATCATATGCCAAGTACGCCCCCTA |
| | TTGACGTCAATGACGGTAAATGGCCCGCCTGGCATTATGCCCAGTACA |
| | TGACCTTATGGGACTTTCCTACTTGGCAGTACATCTACGTATTAGTCAT |
| | CGCTATTACCATGGTCGAGGTGAGCCCCACGTTCTGCTTCACTCTCCCC |
| | ATCTCCCCCCCCTCCCCACCCCCAATTTTGTATTTATTTATTTTTTAATT |
| | ATTTTGTGCAGCGATGGGGGCGGGGGGGGGGGGGGGGCGCGCGCCGG |
| | GCGGGGCGGGGCGGGGCGAGGGGGGGGCGGGGCGAGGCGGAGAGG |
| | TGCGGCGGCAGCCAATCAGAGCGGCGCGCTCCGAAAGTTTCCTTTTAT |
| | GGCGAGGCGGCGGCGGCGGCGGCCCTATAAAAAGCGAAGCGCGCGGC |
| | GGGCGGGAGTCGCTGCGCGCTGCCTTCGCCCCGTGCCCCGCTCCGCCG |
| | CCGCCTCGCGCCGCCCGCCCCGGCTCTGACTGACCGCGTTACTCCCAC |
| | AGGTGAGCGGGCGGGACGGCCCTTCTCCTCCGGGCTGTAATTAGCGCT |
| | TGGTTTAATGACGGCTTGTTTCTTTTCTGTGGCTGCGTGAAAGCCTTGA |
| | GGGGCTCCGGGAGGGCCCTTTGTGCGGGGGGAGCGGCTCGGGGCTGTC |
| | CGCGGGGGGACGGCTGCCTTCGGGGGGGACGGGGCAGGGCGGGGTTC |
| | GGCTTCTGGCGTGTGACCGGCGGCTCTAGAGCCTCTGCTAACCATGTTC |
| | ATGCCTTCTTCTTTTTCCTACAGCTCCTGGGCAACGTGCTGGTTATTGT |
| | GCTGTCTCATCATTTTGGCAAAGAATTGGATCCGCCACCATGGTAAAG |
| | CGTGAGAAAAATGTCATCTATGGCCCTGAGCCTCTCCATCCTTTGGAG |
| | GATTTGACTGCCGGCGAAATGCTGTTTCGTGCTCTCCGCAAGCACTCTC |
| | ATTTGCCTCAAGCCTTGGTCGATGTGGTCGGCGATGAATCTTTGAGCTA |
| | CAAGGAGTTTTTTGAGGCAACCGTCTTGCTGGCTCAGTCCCTCCACAAT |
| | TGTGGCTACAAGATGAACGACGTCGTTAGTATCTGTGCTGAAAACAAT |
| | ACCCGTTTCTTCATTCCAGTCATCGCCGCATGGTATATCGGTATGATCG |
| | TGGCTCCAGTCAACGAGAGCTACATTCCCGACGAACTGTGTAAAGTCA |
| | TGGGTATCTCTAAGCCACAGATTGTCTTCACCACTAAGAATATTCTGAA |
| | CAAAGTCCTGGAAGTCCAAAGCCGCACCAACTTTATTAAGCGTATCAT |
| | CATCTTGGACACTGTGGAGAATATTCACGGTTGCGAATCTTTGCCTAAT |
| | TTCATCTCTCGCTATTCAGACGGCAACATCGCAAACTTTAAACCACTCC |
| | ACTTCGACCCTGTGGAACAAGTTGCAGCCATTCTGTGTAGCAGCGGTA |
| | CTACTGGACTCCCAAAGGGAGTCATGCAGACCCATCAAAACATTTGCG |
| | TGCGTCTGATCCATGCTCTCGATCCACGCTACGGCACTCAGCTGATTCC |
| | TGGTGTCACCGTCTTGGTCTACTTGCCTTTCTTCCATGCTTTCGGCTTTC |
| | ATATTACTTTGGGTTACTTTATGGTCGGTCTCCGCGTGATTATGTTCCG |
| | CCGTTTTGATCAGGAGGCTTTCTTGAAAGCCATCCAAGATTATGAAGT |
| | CCGCAGTGTCATCAACGTGCCTAGCGTGATCCTGTTTTTGTCTAAGAGC |
| | CCACTCGTGGACAAGTACGACTTGTCTTCACTGCGTGAATTGTGTTGCG |
| | GTGCCGCTCCACTGGCTAAGGAGGTCGCTGAAGTGGCCGCCAAACGCT |
| | TGAATCTTCCAGGGATTCGTTGTGGCTTCGGCCTCACCGAATCTACCAG |
| | TGCGATTATCCAGACTCTCGGGGATGAGTTTAAGAGCGGCTCTTTGGG |
| | CCGTGTCACTCCACTCATGGCTGCTAAGATCGCTGATCGCGAAACTGG |
| | TAAGGCTTTGGGCCCGAACCAAGTGGGCGAGCTGTGTATCAAAGGCCC |
| | TATGGTGAGCAAGGGTTATGTCAATAACGTTGAAGCTACCAAGGAGGC |
| | CATCGACGACGACGGCTGGTTGCATTCTGGTGATTTTGGATATTACGA |
| | CGAAGATGAGCATTTTTACGTCGTGGATCGTTACAAGGAGCTGATCAA |
| | ATACAAGGGTAGCCAGGTTGCTCCAGCTGAGTTGGAGGAGATTCTGTT |
| | GAAAAATCCATGCATTCGCGATGTCGCTGTGGTCGGCATTCCTGATCT |
| | GGAGGCCGGCGAACTGCCTTCTGCTTTCGTTGTCAAGCAGCCTGGTAC |
| | AGAAATTACCGCCAAAGAAGTGTATGATTACCTGGCTGAACGTGTGAG |
| | CCATACTAAGTACTTGCGTGGCGGCGTGCGTTTTGTTGACTCCATCCCT |
| | CGTAACGTAACAGGCAAAATTACCCGCAAGGAGCTGTTGAAACAATTG |
| | TTGGTGAAGGCCGGCGGTGGATCCGGAGAGGGCAGAGGAAGTCTTCT |
| | AACATGCGGTGACGTGGAGGAGAATCCCGGCCCTATGGTGAGCAAGG |
| | GCGAGGAGCTGTTCACCGGGGTGGTGCCCATCCTGGTCGAGCTGGACG |
| | GCGACGTAAACGGCCACAAGTTCAGCGTGTCCGGCGAGGGCGAGGGC |
| | GATGCCACCTACGGCAAGCTGACCCTGAAGTTCATTTGCACCACCGGC |
| | AAGCTGCCCGTGCCCTGGCCCACCCTCGTGACCACCCTGACCTACGGC |
| | GTGCAGTGCTTCAGCCGCTACCCCGACCACATGAAGCAGCACGACTTC |
| | TTCAAGTCCGCCATGCCCGAAGGCTACGTCCAGGAGCGCACCATCTTC |
| | TTCAAGGACGACGGCAACTACAAGACCCGCGCCGAGGTGAAGTTCGA |
| | GGGCGACACCCTGGTGAACCGCATCGAGCTGAAGGGCATCGACTTCAA |
| | GGAGGACGGCAACATCCTGGGGCACAAGCTGGAGTACAACTACAACA |
| | GCCACAACGTCTATATCATGGCCGACAAGCAGAAGAACGGCATCAAG |
| | GTGAACTTCAAGATCCGCCACAACATCGAGGACGGCAGCGTGCAGCTC |
| | GCCGACCACTACCAGCAGAACACCCCCATCGGCGACGGCCCCGTGCTG |
| | CTGCCCGACAACCACTACCTGAGCACCCAGTCCGCCCTGAGCAAAGAC |
| | CCCAACGAGAAGCGCGATCACATGGTCCTGCTGGAGTTCGTGACCGCC |
| | GCCGGGATCACTCTCGGCATGGACGAGCTGTACAAGTAAAATCAACCT |
| | CTGGATTACAAAATTTGTGAAAGATTGACTGGTATTCTTAACTATGTTG |
| | CTCCTTTTACGCTATGTGGATACGCTGCTTTAATGCCTTTGTATCATGCT |
| | ATTGCTTCCCGTATGGCTTTCATTTTCTCCTCCTTGTATAAATCCTGGTT |
| | AGTTCTTGCCACGGCGGAACTCATCGCCGCCTGCCTTGCCCGCTGCTGG |
| | ACAGGGGCTCGGCTGTTGGGCACTGACAATTCCGTGGTGTTTATTTGTG |
| | AAATTTGTGATGCTATTGCTTTATTTGTAACCATCTAGCTTTATTTGTGA |
| | AATTTGTGATGCTATTGCTTTATTTGTAACCATTATAAGCTGCAATAAA |
| | CAAGTTTCTTCCTAGAGCATGGCTACGTAGATAAGTAGCATGGCGGGT |
| | TAATCATTAACTACAAGGAACCCCTAGTGATGGAGTTGGCCACTCCCT |
| | CTCTGCGCGCTCGCTCGCTCACTGAGGCCGGGCGACCAAAGGTCGCCC |
| | GACGCCCGGGCTTTGCCCGGGCGGCCTCAGTGAGCGAGCGAGCGCGC |
| | AGCTGAGTGAGGCCGCCGCTGAGGGAGTACAGCAGCGGGAGCGCCAC |
| | C |
| 10 | CATTAATGAATCGGCCAACGCGCGGGGAGAGGCGGTTTGCGTATTGGG |
| AAV H- | CGCTCTTCCGCTTCCTCGCTCACTGACTCGCTGCGCTCGGTCGTTCGGC |
| CDKN2A | TGCGGCGAGCGGTATCAGCTCACTCAAAGGCGGTAATACGGTTATCCA |
| OSK | CAGAATCAGGGGATAACGCAGGAAAGAACATGTGAGCAAAAGGCCAG |
| shortWPRE | CAAAAGGCCAGGAACCGTAAAAAGGCCGCGTTGCTGGCGTTTTTCCAT |
| sv40 | AGGCTCCGCCCCCCTGACGAGCATCACAAAAATCGACGCTCAAGTCAG |
| polya | AGGTGGCGAAACCCGACAGGACTATAAAGATACCAGGCGTTTCCCCCT |
| nucleic | GGAAGCTCCCTCGTGCGCTCTCCTGTTCCGACCCTGCCGCTTACCGGAT |
| acid | ACCTGTCCGCCTTTCTCCCTTCGGGAAGCGTGGCGCTTTCTCATAGCTC |
| sequence | ACGCTGTAGGTATCTCAGTTCGGTGTAGGTCGTTCGCTCCAAGCTGGG |
| | CTGTGTGCACGAACCCCCCGTTCAGCCCGACCGCTGCGCCTTATCCGGT |
| | AACTATCGTCTTGAGTCCAACCCGGTAAGACACGACTTATCGCCACTG |
| | GCAGCAGCCACTGGTAACAGGATTAGCAGAGCGAGGTATGTAGGCGG |
| | TGCTACAGAGTTCTTGAAGTGGTGGCCTAACTACGGCTACACTAGAAG |
| | AACAGTATTTGGTATCTGCGCTCTGCTGAAGCCAGTTACCTTCGGAAA |
| | AAGAGTTGGTAGCTCTTGATCCGGCAAACAAACCACCGCTGGTAGCGG |
| | TGGTTTTTTTGTTTGCAAGCAGCAGATTACGCGCAGAAAAAAAGGATC |
| | TCAAGAAGATCCTTTGATCTTTTCTACGGGGTCTGACGCTCAGTGGAAC |
| | GAAAACTCACGTTAAGGGATTTTGGTCATGAGATTATCAAAAAGGATC |
| | TTCACCTAGATCCTTTTAAATTAAAAATGAAGTTTTAAATCAATCTAAA |
| | GTATATATGAGTAAACTTGGTCTGACAGTTACCAATGCTTAATCAGTG |
| | AGGCACCTATCTCAGCGATCTGTCTATTTCGTTCATCCATAGTTGCCTG |
| | ACTCCCCGTCGTGTAGATAACTACGATACGGGAGGGCTTACCATCTGG |
| | CCCCAGTGCTGCAATGATACCGCGAGACCCACGCTCACCGGCTCCAGA |
| | TTTATCAGCAATAAACCAGCCAGCCGGAAGGGCCGAGCGCAGAAGTG |
| | GTCCTGCAACTTTATCCGCCTCCATCCAGTCTATTAATTGTTGCCGGGA |
| | AGCTAGAGTAAGTAGTTCGCCAGTTAATAGTTTGCGCAACGTTGTTGC |
| | CATTGCTACAGGCATCGTGGTGTCACGCTCGTCGTTTGGTATGGCTTCA |
| | TTCAGCTCCGGTTCCCAACGATCAAGGCGAGTTACATGATCCCCCATG |
| | TTGTGCAAAAAAGCGGTTAGCTCCTTCGGTCCTCCGATCGTTGTCAGA |
| | AGTAAGTTGGCCGCAGTGTTATCACTCATGGTTATGGCAGCACTGCAT |
| | AATTCTCTTACTGTCATGCCATCCGTAAGATGCTTTTCTGTGACTGGTG |
| | AGTACTCAACCAAGTCATTCTGAGAATAGTGTATGCGGCGACCGAGTT |
| | GCTCTTGCCCGGCGTCAATACGGGATAATACCGCGCCACATAGCAGAA |
| | CTTTAAAAGTGCTCATCATTGGAAAACGTTCTTCGGGGCGAAAACTCT |
| | CAAGGATCTTACCGCTGTTGAGATCCAGTTCGATGTAACCCACTCGTG |
| | CACCCAACTGATCTTCAGCATCTTTTACTTTCACCAGCGTTTCTGGGTG |
| | AGCAAAAACAGGAAGGCAAAATGCCGCAAAAAAGGGAATAAGGGCG |
| | ACACGGAAATGTTGAATACTCATACTCTTCCTTTTTCAATATTATTGAA |
| | GCATTTATCAGGGTTATTGTCTCATGAGCGGATACATATTTGAATGTAT |
| | TTAGAAAAATAAACAAATAGGGGTTCCGCGCACATTTCCCCGAAAAGT |
| | GCCACCTAAATTGTAAGCGTTAATATTTTGTTAAAATTCGCGTTAAATT |
| | TTTGTTAAATCAGCTCATTTTTTAACCAATAGGCCGAAATCGGCAAAAT |
| | CCCTTATAAATCAAAAGAATAGACCGAGATAGGGTTGAGTGTTGTTCC |
| | AGTTTGGAACAAGAGTCCACTATTAAAGAACGTGGACTCCAACGTCAA |
| | AGGGCGAAAAACCGTCTATCAGGGCGATGGCCCACTACGTGAACCATC |
| | ACCCTAATCAAGTTTTTTGGGGTCGAGGTGCCGTAAAGCACTAAATCG |
| | GAACCCTAAAGGGAGCCCCCGATTTAGAGCTTGACGGGGAAAGCCGG |
| | CGAACGTGGCGAGAAAGGAAGGGAAGAAAGCGAAAGGAGCGGGCGC |
| | TAGGGCGCTGGCAAGTGTAGCGGTCACGCTGCGCGTAACCACCACACC |
| | CGCCGCGCTTAATGCGCCGCTACAGGGCGCGTCCCATTCGCCATTCAG |
| | GCTGCGCAACTGTTGGGAAGGGCGATCGGTGCGGGCCTCTTCGCTATT |
| | ACGCCAGCTGCGCGCTCGCTCGCTCACTGAGGCCGCCCGGGCAAAGCC |
| | CGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTT |
| | AATGATTAACCCGCCATGCTACTTATCTACGTAGCCATGCTCTAGGAA |
| | GAGCGGGCCACATTTCCAGGTTCCCGGAGCTGGGCCTAGTCCCGAATC |
| | CTCTGGCACACACCCACCCACTCAGGCCGCGGGTCCAGCCCGCGAGGT |
| | TTAGGACGGATCCAGGCAGACCGCAGGCTCCGGGTCGGGGCACCGGG |
| | TCAGCGCGCCGGCCTGAAGGCGGCGTCCTGGGCTCGACTTCCCGCGCG |
| | CGGAGAGCCGGCGAGCCCGCGTCCGAGTTCCTGGACGAGAGCCGAGC |
| | CTCGCTTAGACCGCGCTCAGGACCCGGCTCCTCCGCATTCTCCGGCTGC |
| | CCCTGTGTCCTCGACTCACCCCTCCTTTCTGCCGCTCCTTCCTTTCCTTG |
| | CCCTGCTTTTACTGTTCCCAAACAGGACCGCTTTTCCTGTCTCCCAGCT |
| | GGAAAGGAGGAAGGGAGAGAGTCCAGAAAGGATCGGTGATGTGGAA |
| | GAAAAGGGGAGGAGGGGACATGGAGGGGGAGACCGGAGAGAGAACG |
| | TACGCCGAGGAGTCAGGCGGCGGGATCAAGGGGAGTCGGGGTGTCTG |
| | GGCGCGGGGCAGAGCGTGGAGGCGGCAGCGGCCAACGGTCGCCAAGA |
| | CAACCATTCTACGCGAGGACGCGGCGACAGGAGGGGAGCGGCCAGCA |
| | GGGGAGGGGAGCGCGGGGGAAGAGGAAAGAGGAAGAAGCGCTCAGA |
| | TGCTCCGCGGCTGTCGTGAAGGTTAAAACCGAAAATAAAAATGGGCTA |
| | GACACAAAGGACTCGGTGCTTGTCCCAGCCAGGCGCCCTCGGCGACGC |
| | GGGCAGCTGGGAGGGGAATGGGCGCCCGGACCCAGCTGGGACCCCCG |
| | GGTGCGACTCCACCTACCTAGTCCGGCGCCAGGCCGGGTCGACAGCTC |
| | CGGCAGCGCCAGCGCCGCGCCGTGTCCAGATGTCGCGTCAGAGGCGTG |
| | CAGCGGTTTAGTTTAATTTCGCTTGTTTTCCAAATCTAGAAGAGGAGCG |
| | GAGCGGCTTTTAGTTCAAAACTGACATTCAGCCTCCTGATTGGCGGAT |
| | AGAGCAATGAGATGACCTCGCTTTCCTTTCTTCCTTTTTCATTTTTAAAT |
| | AATCTAGTTTGAAGAATGGAAGACTTTCGACGAGGGGAGCCAGGAAT |
| | AAAATAAGGGGAATAGGGGAGCGGGGACGCGAGCAGCACCAGAATCC |
| | GCGGGAGCGCGGCTGTTCCTGGTAGGGCCGTGTCAGGTGACGGATGTA |
| | GCTAGGGGGCGAGCTGCCTGGAGTTGCGTTCCAGGCGTCCGGCCCCTG |
| | GGCCGTCACCGCGGGGCGCCCGCGCTGAGGGTGGGAAGATGGTGGTG |
| | GGGGTGGGGGCGCACACAGGGCGGGAAAGTGGCGGTAGGCGGGAGG |
| | GAGAGGAACGCGGGCCCTGAGCCGCCCGCGCGCGCGCCTCCCTACGG |
| | GCGCCTCCGGCAGCCCTTCCCGCGTGCGCAGGGCTCAGAGCCGTTCCG |
| | AGATCTTGGAGGTCCGGGTGGGAGTGGGGGTGGGGTGGGGGTGGGGG |
| | TGAAGGTGGGGGGCGGGCGCGCTCAGGGAAGGCGGGTGCGCGCCTGC |
| | GGGGCGGAGATGGGCAGGGGGCGGTGCGTGGGTCCCAGTCTGCAGTT |
| | AAGGGGGCAGGAGTGGCGCTGCTCACCTCTGGTGCCAAAGGGCGGCG |
| | CAGCGGCTGCCGAGCTCGGCCCTGGAGGCGGCGAGAACATGGTGATG |
| | GCGGGACACCTGGCTTCGGATTTCGCCTTCTCGCCCCCTCCAGGTGGTG |
| | GAGGTGATGGGCCAGGGGGGCCGGAGCCGGGCTGGGTTGATCCTCGG |
| | ACCTGGCTAAGCTTCCAAGGCCCTCCTGGAGGGCCAGGAATCGGGCCG |
| | GGGGTTGGGCCAGGCTCTGAGGTGTGGGGGATTCCCCCATGCCCCCCG |
| | CCGTATGAGTTCTGTGGGGGGATGGCGTACTGTGGGCCCCAGGTTGGA |
| | GTGGGGCTAGTGCCCCAAGGCGGCTTGGAGACCTCTCAGCCTGAGGGC |
| | GAAGCAGGAGTCGGGGTGGAGAGCAACTCCGATGGGGCCTCCCCGGA |
| | GCCCTGCACCGTCACCCCTGGTGCCGTGAAGCTGGAGAAGGAGAAGCT |
| | GGAGCAAAACCCGGAGGAGTCCCAGGACATCAAAGCTCTGCAGAAAG |
| | AACTCGAGCAATTTGCCAAGCTCCTGAAGCAGAAGAGGATCACCCTGG |
| | GATATACACAGGCCGATGTGGGGCTCACCCTGGGGGTTCTATTTGGGA |
| | AGGTATTCAGCCAAACGACCATCTGCCGCTTTGAGGCTCTGCAGCTTA |
| | GCTTCAAGAACATGTGTAAGCTGCGGCCCTTGCTGCAGAAGTGGGTGG |
| | AGGAAGCTGACAACAATGAAAATCTTCAGGAGATATGCAAAGCAGAA |
| | ACCCTCGTGCAGGCCCGAAAGAGAAAGCGAACCAGTATCGAGAACCG |
| | AGTGAGAGGCAACCTGGAGAATTTGTTCCTGCAGTGCCCGAAACCCAC |
| | ACTGCAGCAGATCAGCCACATCGCCCAGCAGCTTGGGCTCGAGAAGG |
| | ATGTGGTCCGAGTGTGGTTCTGTAACCGGCGCCAGAAGGGCAAGCGAT |
| | CAAGCAGCGACTATGCACAACGAGAGGATTTTGAGGCTGCTGGGTCTC |
| | CTTTCTCAGGGGGACCAGTGTCCTTTCCTCTGGCCCCAGGGCCCCATTT |
| | TGGTACCCCAGGCTATGGGAGCCCTCACTTCACTGCACTGTACTCCTCG |
| | GTCCCTTTCCCTGAGGGGGAAGCCTTTCCCCCTGTCTCCGTCACCACTC |
| | TGGGCTCTCCCATGCATTCAAACGGATCCGGAGCCACGAACTTCTCTCT |
| | GTTAAAGCAAGCAGGAGATGTTGAAGAAAACCCCGGGCCTATGTACA |
| | ACATGATGGAGACGGAGCTGAAGCCGCCGGGCCCGCAGCAAACTTCG |
| | GGGGGCGGCGGCGGCAACTCCACCGCGGCGGCGGCCGGCGGCAACCA |
| | GAAAAACAGCCCGGACCGCGTCAAGCGGCCCATGAATGCCTTCATGGT |
| | GTGGTCCCGCGGGCAGCGGCGCAAGATGGCCCAGGAGAACCCCAAGA |
| | TGCACAACTCGGAGATCAGCAAGCGCCTGGGCGCCGAGTGGAAACTTT |
| | TGTCGGAGACGGAGAAGCGGCCGTTCATCGACGAGGCTAAGCGGCTG |
| | CGAGCGCTGCACATGAAGGAGCACCCGGATTATAAATACCGGCCCCG |
| | GCGGAAAACCAAGACGCTCATGAAGAAGGATAAGTACACGCTGCCCG |
| | GCGGGCTGCTGGCCCCCGGCGGCAATAGCATGGCGAGCGGGGTCGGG |
| | GTGGGCGCCGGCCTGGGCGCGGGCGTGAACCAGCGCATGGACAGTTA |
| | CGCGCACATGAACGGCTGGAGCAACGGCAGCTACAGCATGATGCAGG |
| | ACCAGCTGGGCTACCCGCAGCACCCGGGCCTCAATGCGCACGGCGCAG |
| | CGCAGATGCAGCCCATGCACCGCTACGACGTGAGCGCCCTGCAGTACA |
| | ACTCCATGACCAGCTCGCAGACCTACATGAACGGCTCGCCCACCTACA |
| | GCATGTCCTACTCGCAGCAGGGCACCCCTGGCATGGCTCTTGGCTCCA |
| | TGGGTTCGGTGGTCAAGTCCGAGGCCAGCTCCAGCCCCCCTGTGGTTA |
| | CCTCTTCCTCCCACTCCAGGGCGCCCTGCCAGGCCGGGGACCTCCGGG |
| | ACATGATCAGCATGTATCTCCCCGGCGCCGAGGTGCCGGAACCCGCCG |
| | CCCCCAGCAGACTTCACATGTCCCAGCACTACCAGAGCGGCCCGGTGC |
| | CCGGCACGGCCATTAACGGCACACTGCCCCTCTCACACATGGGATCCG |
| | GAGCCACGAACTTCTCTCTGTTAAAGCAAGCAGGAGATGTTGAAGAAA |
| | ACCCCGGGCCTATGGCTGTCAGCGACGCGCTGCTCCCATCTTTCTCCA |
| | CGTTCGCGTCTGGCCCGGCGGGAAGGGAGAAGACACTGCGTCAAGCA |
| | GGTGCCCCGAATAACCGCTGGCGGGAGGAGCTCTCCCACATGAAGCG |
| | ACTTCCCCCAGTGCTTCCCGGCCGCCCCTATGACCTGGCGGCGGCGAC |
| | CGTGGCCACAGACCTGGAGAGCGGCGGAGCCGGTGCGGCTTGCGGCG |
| | GTAGCAACCTGGCGCCCCTACCTCGGAGAGAGACCGAGGAGTTCAAC |
| | GATCTCCTGGACCTGGACTTTATTCTCTCCAATTCGCTGACCCATCCTC |
| | CGGAGTCAGTGGCCGCCACCGTGTCCTCGTCAGCGTCAGCCTCCTCTTC |
| | GTCGTCGCCGTCGAGCAGCGGCCCTGCCAGCGCGCCCTCCACCTGCAG |
| | CTTCACCTATCCGATCCGGGCCGGGAACGACCCGGGCGTGGCGCCGGG |
| | CGGCACGGGCGGAGGCCTCCTCTATGGCAGGGAGTCCGCTCCCCCTCC |
| | GACGGCTCCCTTCAACCTGGCGGACATCAACGACGTGAGCCCCTCGGG |
| | CGGCTTCGTGGCCGAGCTCCTGCGGCCAGAATTGGACCCGGTGTACAT |
| | TCCGCCGCAGCAGCCGCAGCCGCCAGGTGGCGGGCTGATGGGCAAGTT |
| | CGTGCTGAAGGCGTCGCTGAGCGCCCCTGGCAGCGAGTACGGCAGCCC |
| | GTCGGTCATCAGCGTCAGCAAAGGCAGCCCTGACGGCAGCCACCCGGT |
| | GGTGGTGGCGCCCTACAACGGCGGGCCGCCGCGCACGTGCCCCAAGAT |
| | CAAGCAGGAGGCGGTCTCTTCGTGCACCCACTTGGGCGCTGGACCCCC |
| | TCTCAGCAATGGCCACCGGCCGGCTGCACACGACTTCCCCCTGGGGCG |
| | GCAGCTCCCCAGCAGGACTACCCCGACCCTGGGTCTTGAGGAAGTGCT |
| | GAGCAGCAGGGACTGTCACCCTGCCCTGCCGCTTCCTCCCGGCTTCCAT |
| | CCCCACCCGGGGCCCAATTACCCATCCTTCCTGCCCGATCAGATGCAG |
| | CCGCAAGTCCCGCCGCTCCATTACCAAGAGCTCATGCCACCCGGTTCC |
| | TGCATGCCAGAGGAGCCCAAGCCAAAGAGGGGAAGACGATCGTGGCC |
| | CCGGAAAAGGACCGCCACCCACACTTGTGATTACGCGGGCTGCGGCAA |
| | AACCTACACAAAGAGTTCCCATCTCAAGGCACACCTGCGAACCCACAC |
| | AGGTGAGAAACCTTACCACTGTGACTGGGACGGCTGTGGATGGAAATT |
| | CGCCCGCTCAGATGAACTGACCAGGCACTACCGTAAACACACGGGGC |
| | ACCGCCCGTTCCAGTGCCAAAAATGCGACCGAGCATTTTCCAGGTCGG |
| | ACCACCTCGCCTTACACATGAAGAGGCATTTTTAAAATCAACCTCTGG |
| | ATTACAAAATTTGTGAAAGATTGACTGGTATTCTTAACTATGTTGCTCC |
| | TTTTACGCTATGTGGATACGCTGCTTTAATGCCTTTGTATCATGCTATT |
| | GCTTCCCGTATGGCTTTCATTTTCTCCTCCTTGTATAAATCCTGGTTAGT |
| | TCTTGCCACGGCGGAACTCATCGCCGCCTGCCTTGCCCGCTGCTGGAC |
| | AGGGGCTCGGCTGTTGGGCACTGACAATTCCGTGGTGTTTATTTGTGA |
| | AATTTGTGATGCTATTGCTTTATTTGTAACCATCTAGCTTTATTTGTGAA |
| | ATTTGTGATGCTATTGCTTTATTTGTAACCATTATAAGCTGCAATAAAC |
| | AAGTTTCTTCCTAGAGCATGGCTACGTAGATAAGTAGCATGGCGGGTT |
| | AATCATTAACTACAAGGAACCCCTAGTGATGGAGTTGGCCACTCCCTC |
| | TCTGCGCGCTCGCTCGCTCACTGAGGCCGGGCGACCAAAGGTCGCCCG |
| | ACGCCCGGGCTTTGCCCGGGCGGCCTCAGTGAGCGAGCGAGCGCGCA |
| | GCTG |
| 11 | CATTAATGAATCGGCCAACGCGCGGGGAGAGGCGGTTTGCGTATTGGG |
| AAV H- | CGCTCTTCCGCTTCCTCGCTCACTGACTCGCTGCGCTCGGTCGTTCGGC |
| CDKN2A | TGCGGCGAGCGGTATCAGCTCACTCAAAGGCGGTAATACGGTTATCCA |
| IKKB sup | CAGAATCAGGGGATAACGCAGGAAAGAACATGTGAGCAAAAGGCCAG |
| repressor- | CAAAAGGCCAGGAACCGTAAAAAGGCCGCGTTGCTGGCGTTTTTCCAT |
| GFP | AGGCTCCGCCCCCCTGACGAGCATCACAAAAATCGACGCTCAAGTCAG |
| shortWPRE | AGGTGGCGAAACCCGACAGGACTATAAAGATACCAGGCGTTTCCCCCT |
| sv40 | GGAAGCTCCCTCGTGCGCTCTCCTGTTCCGACCCTGCCGCTTACCGGAT |
| polya | ACCTGTCCGCCTTTCTCCCTTCGGGAAGCGTGGCGCTTTCTCATAGCTC |
| nucleic | ACGCTGTAGGTATCTCAGTTCGGTGTAGGTCGTTCGCTCCAAGCTGGG |
| acid | CTGTGTGCACGAACCCCCCGTTCAGCCCGACCGCTGCGCCTTATCCGGT |
| sequence | AACTATCGTCTTGAGTCCAACCCGGTAAGACACGACTTATCGCCACTG |
| | GCAGCAGCCACTGGTAACAGGATTAGCAGAGCGAGGTATGTAGGCGG |
| | TGCTACAGAGTTCTTGAAGTGGTGGCCTAACTACGGCTACACTAGAAG |
| | AACAGTATTTGGTATCTGCGCTCTGCTGAAGCCAGTTACCTTCGGAAA |
| | AAGAGTTGGTAGCTCTTGATCCGGCAAACAAACCACCGCTGGTAGCGG |
| | TGGTTTTTTTGTTTGCAAGCAGCAGATTACGCGCAGAAAAAAAGGATC |
| | TCAAGAAGATCCTTTGATCTTTTCTACGGGGTCTGACGCTCAGTGGAAC |
| | GAAAACTCACGTTAAGGGATTTTGGTCATGAGATTATCAAAAAGGATC |
| | TTCACCTAGATCCTTTTAAATTAAAAATGAAGTTTTAAATCAATCTAAA |
| | GTATATATGAGTAAACTTGGTCTGACAGTTACCAATGCTTAATCAGTG |
| | AGGCACCTATCTCAGCGATCTGTCTATTTCGTTCATCCATAGTTGCCTG |
| | ACTCCCCGTCGTGTAGATAACTACGATACGGGAGGGCTTACCATCTGG |
| | CCCCAGTGCTGCAATGATACCGCGAGACCCACGCTCACCGGCTCCAGA |
| | TTTATCAGCAATAAACCAGCCAGCCGGAAGGGCCGAGCGCAGAAGTG |
| | GTCCTGCAACTTTATCCGCCTCCATCCAGTCTATTAATTGTTGCCGGGA |
| | AGCTAGAGTAAGTAGTTCGCCAGTTAATAGTTTGCGCAACGTTGTTGC |
| | CATTGCTACAGGCATCGTGGTGTCACGCTCGTCGTTTGGTATGGCTTCA |
| | TTCAGCTCCGGTTCCCAACGATCAAGGCGAGTTACATGATCCCCCATG |
| | TTGTGCAAAAAAGCGGTTAGCTCCTTCGGTCCTCCGATCGTTGTCAGA |
| | AGTAAGTTGGCCGCAGTGTTATCACTCATGGTTATGGCAGCACTGCAT |
| | AATTCTCTTACTGTCATGCCATCCGTAAGATGCTTTTCTGTGACTGGTG |
| | AGTACTCAACCAAGTCATTCTGAGAATAGTGTATGCGGCGACCGAGTT |
| | GCTCTTGCCCGGCGTCAATACGGGATAATACCGCGCCACATAGCAGAA |
| | CTTTAAAAGTGCTCATCATTGGAAAACGTTCTTCGGGGCGAAAACTCT |
| | CAAGGATCTTACCGCTGTTGAGATCCAGTTCGATGTAACCCACTCGTG |
| | CACCCAACTGATCTTCAGCATCTTTTACTTTCACCAGCGTTTCTGGGTG |
| | AGCAAAAACAGGAAGGCAAAATGCCGCAAAAAAGGGAATAAGGGCG |
| | ACACGGAAATGTTGAATACTCATACTCTTCCTTTTTCAATATTATTGAA |
| | GCATTTATCAGGGTTATTGTCTCATGAGCGGATACATATTTGAATGTAT |
| | TTAGAAAAATAAACAAATAGGGGTTCCGCGCACATTTCCCCGAAAAGT |
| | GCCACCTAAATTGTAAGCGTTAATATTTTGTTAAAATTCGCGTTAAATT |
| | TTTGTTAAATCAGCTCATTTTTTAACCAATAGGCCGAAATCGGCAAAAT |
| | CCCTTATAAATCAAAAGAATAGACCGAGATAGGGTTGAGTGTTGTTCC |
| | AGTTTGGAACAAGAGTCCACTATTAAAGAACGTGGACTCCAACGTCAA |
| | AGGGCGAAAAACCGTCTATCAGGGCGATGGCCCACTACGTGAACCATC |
| | ACCCTAATCAAGTTTTTTGGGGTCGAGGTGCCGTAAAGCACTAAATCG |
| | GAACCCTAAAGGGAGCCCCCGATTTAGAGCTTGACGGGGAAAGCCGG |
| | CGAACGTGGCGAGAAAGGAAGGGAAGAAAGCGAAAGGAGCGGGCGC |
| | TAGGGCGCTGGCAAGTGTAGCGGTCACGCTGCGCGTAACCACCACACC |
| | CGCCGCGCTTAATGCGCCGCTACAGGGCGCGTCCCATTCGCCATTCAG |
| | GCTGCGCAACTGTTGGGAAGGGCGATCGGTGCGGGCCTCTTCGCTATT |
| | ACGCCAGCTGCGCGCTCGCTCGCTCACTGAGGCCGCCCGGGCAAAGCC |
| | CGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTT |
| | AATGATTAACCCGCCATGCTACTTATCTACGTAGCCATGCTCTAGGAA |
| | GAGCGGGCCACATTTCCAGGTTCCCGGAGCTGGGCCTAGTCCCGAATC |
| | CTCTGGCACACACCCACCCACTCAGGCCGCGGGTCCAGCCCGCGAGGT |
| | TTAGGACGGATCCAGGCAGACCGCAGGCTCCGGGTCGGGGCACCGGG |
| | TCAGCGCGCCGGCCTGAAGGCGGCGTCCTGGGCTCGACTTCCCGCGCG |
| | CGGAGAGCCGGCGAGCCCGCGTCCGAGTTCCTGGACGAGAGCCGAGC |
| | CTCGCTTAGACCGCGCTCAGGACCCGGCTCCTCCGCATTCTCCGGCTGC |
| | CCCTGTGTCCTCGACTCACCCCTCCTTTCTGCCGCTCCTTCCTTTCCTTG |
| | CCCTGCTTTTACTGTTCCCAAACAGGACCGCTTTTCCTGTCTCCCAGCT |
| | GGAAAGGAGGAAGGGAGAGAGTCCAGAAAGGATCGGTGATGTGGAA |
| | GAAAAGGGGAGGAGGGGACATGGAGGGGGAGACCGGAGAGAGAACG |
| | TACGCCGAGGAGTCAGGCGGCGGGATCAAGGGGAGTCGGGGTGTCTG |
| | GGCGCGGGGCAGAGCGTGGAGGCGGCAGCGGCCAACGGTCGCCAAGA |
| | CAACCATTCTACGCGAGGACGCGGCGACAGGAGGGGAGCGGCCAGCA |
| | GGGGAGGGGAGCGCGGGGGAAGAGGAAAGAGGAAGAAGCGCTCAGA |
| | TGCTCCGCGGCTGTCGTGAAGGTTAAAACCGAAAATAAAAATGGGCTA |
| | GACACAAAGGACTCGGTGCTTGTCCCAGCCAGGCGCCCTCGGCGACGC |
| | GGGCAGCTGGGAGGGGAATGGGCGCCCGGACCCAGCTGGGACCCCCG |
| | GGTGCGACTCCACCTACCTAGTCCGGCGCCAGGCCGGGTCGACAGCTC |
| | CGGCAGCGCCAGCGCCGCGCCGTGTCCAGATGTCGCGTCAGAGGCGTG |
| | CAGCGGTTTAGTTTAATTTCGCTTGTTTTCCAAATCTAGAAGAGGAGCG |
| | GAGCGGCTTTTAGTTCAAAACTGACATTCAGCCTCCTGATTGGCGGAT |
| | AGAGCAATGAGATGACCTCGCTTTCCTTTCTTCCTTTTTCATTTTTAAAT |
| | AATCTAGTTTGAAGAATGGAAGACTTTCGACGAGGGGAGCCAGGAAT |
| | AAAATAAGGGGAATAGGGGAGCGGGGACGCGAGCAGCACCAGAATCC |
| | GCGGGAGCGCGGCTGTTCCTGGTAGGGCCGTGTCAGGTGACGGATGTA |
| | GCTAGGGGGCGAGCTGCCTGGAGTTGCGTTCCAGGCGTCCGGCCCCTG |
| | GGCCGTCACCGCGGGGCGCCCGCGCTGAGGGTGGGAAGATGGTGGTG |
| | GGGGTGGGGGCGCACACAGGGCGGGAAAGTGGCGGTAGGCGGGAGG |
| | GAGAGGAACGCGGGCCCTGAGCCGCCCGCGCGCGCGCCTCCCTACGG |
| | GCGCCTCCGGCAGCCCTTCCCGCGTGCGCAGGGCTCAGAGCCGTTCCG |
| | AGATCTTGGAGGTCCGGGTGGGAGTGGGGGTGGGGTGGGGGTGGGGG |
| | TGAAGGTGGGGGGCGGGCGCGCTCAGGGAAGGCGGGTGCGCGCCTGC |
| | GGGGCGGAGATGGGCAGGGGGCGGTGCGTGGGTCCCAGTCTGCAGTT |
| | AAGGGGGCAGGAGTGGCGCTGCTCACCTCTGGTGCCAAAGGGCGGCG |
| | CAGCGGCTGCCGAGCTCGGCCCTGGAGGCGGCGAGAACATGGTGATG |
| | TTCCAGGCGGCCGAGCGTCCCCAGGAGTGGGCCATGGAGGGCCCCCGC |
| | GACGGGCTGAAGAAGGAGCGGCTACTGGACGACCGCCACGACGCCGG |
| | CCTGGACGCCATGAAAGACGAGGAGTACGAGCAGATGGTCAAGGAGC |
| | TGCAGGAGATCCGCCTCGAGCCGCAGGAGGTGCCGCGCGGCTCGGAG |
| | CCCTGGAAGCAGCAGCTCACCGAGGACGGGGACTCGTTCCTGCACTTG |
| | GCCATCATCCATGAAGAAAAGGCACTGACCATGGAAGTGATCCGCCA |
| | GGTGAAGGGAGACCTGGCCTTCCTCAACTTCCAGAACAACCTGCAGCA |
| | GACTCCACTCCACTTGGCTGTGATCACCAACCAGCCAGAAATTGCTGA |
| | GGCACTTCTGGGAGCTGGCTGTGATCCTGAGCTCCGAGACTTTCGAGG |
| | AAATACCCCCCTACACCTTGCCTGTGAGCAGGGCTGCCTGGCCAGCGT |
| | GGGAGTCCTGACTCAGTCCTGCACCACCCCGCACCTCCACTCCATCCTG |
| | AAGGCTACCAACTACAATGGCCACACGTGTCTACACTTAGCCTCTATC |
| | CATGGCTACCTGGGCATCGTGGAGCTTTTGGTGTCCTTGGGTGCTGATG |
| | TCAATGCTCAGGAGCCCTGTAATGGCCGGACTGCCCTTCACCTCGCAG |
| | TGGACCTGCAAAATCCTGACCTGGTGTCACTCCTGTTGAAGTGTGGGG |
| | CTGATGTCAACAGAGTTACCTACCAGGGCTATTCTCCCTACCAGCTCAC |
| | CTGGGGCCGCCCAAGCACCCGGATACAGCAGCAGCTGGGCCAGCTGA |
| | CACTAGAAAACCTTCAGATGCTGCCAGAGAGTGAGGATGAGGAGAGC |
| | TATGACACAGAGTCAGAGTTCACGGAGTTCACAGAGGACGAGCTGCCC |
| | TATGATGACTGTGTGTTTGGAGGCCAGCGTCTGACGTTAGGATCCGGA |
| | GAGGGCAGAGGAAGTCTTCTAACATGCGGTGACGTGGAGGAGAATCC |
| | CGGCCCTATGGTGAGCAAGGGCGAGGAGCTGTTCACCGGGGTGGTGCC |
| | CATCCTGGTCGAGCTGGACGGCGACGTAAACGGCCACAAGTTCAGCGT |
| | GTCCGGCGAGGGCGAGGGCGATGCCACCTACGGCAAGCTGACCCTGA |
| | AGTTCATTTGCACCACCGGCAAGCTGCCCGTGCCCTGGCCCACCCTCGT |
| | GACCACCCTGACCTACGGCGTGCAGTGCTTCAGCCGCTACCCCGACCA |
| | CATGAAGCAGCACGACTTCTTCAAGTCCGCCATGCCCGAAGGCTACGT |
| | CCAGGAGCGCACCATCTTCTTCAAGGACGACGGCAACTACAAGACCCG |
| | CGCCGAGGTGAAGTTCGAGGGCGACACCCTGGTGAACCGCATCGAGCT |
| | GAAGGGCATCGACTTCAAGGAGGACGGCAACATCCTGGGGCACAAGC |
| | TGGAGTACAACTACAACAGCCACAACGTCTATATCATGGCCGACAAGC |
| | AGAAGAACGGCATCAAGGTGAACTTCAAGATCCGCCACAACATCGAG |
| | GACGGCAGCGTGCAGCTCGCCGACCACTACCAGCAGAACACCCCCATC |
| | GGCGACGGCCCCGTGCTGCTGCCCGACAACCACTACCTGAGCACCCAG |
| | TCCGCCCTGAGCAAAGACCCCAACGAGAAGCGCGATCACATGGTCCTG |
| | CTGGAGTTCGTGACCGCCGCCGGGATCACTCTCGGCATGGACGAGCTG |
| | TACAAGTAAAATCAACCTCTGGATTACAAAATTTGTGAAAGATTGACT |
| | GGTATTCTTAACTATGTTGCTCCTTTTACGCTATGTGGATACGCTGCTTT |
| | AATGCCTTTGTATCATGCTATTGCTTCCCGTATGGCTTTCATTTTCTCCT |
| | CCTTGTATAAATCCTGGTTAGTTCTTGCCACGGCGGAACTCATCGCCGC |
| | CTGCCTTGCCCGCTGCTGGACAGGGGCTCGGCTGTTGGGCACTGACAA |
| | TTCCGTGGTGTTTATTTGTGAAATTTGTGATGCTATTGCTTTATTTGTAA |
| | CCATCTAGCTTTATTTGTGAAATTTGTGATGCTATTGCTTTATTTGTAAC |
| | CATTATAAGCTGCAATAAACAAGTTTCTTCCTAGAGCATGGCTACGTA |
| | GATAAGTAGCATGGCGGGTTAATCATTAACTACAAGGAACCCCTAGTG |
| | ATGGAGTTGGCCACTCCCTCTCTGCGCGCTCGCTCGCTCACTGAGGCCG |
| | GGCGACCAAAGGTCGCCCGACGCCCGGGCTTTGCCCGGGCGGCCTCAG |
| | TGAGCGAGCGAGCGCGCAGCTG |
| 12 | CATTAATGAATCGGCCAACGCGCGGGGAGAGGCGGTTTGCGTATTGGG |
| AAV m- | CGCTCTTCCGCTTCCTCGCTCACTGACTCGCTGCGCTCGGTCGTTCGGC |
| CDKN2A | TGCGGCGAGCGGTATCAGCTCACTCAAAGGCGGTAATACGGTTATCCA |
| h-IKB | CAGAATCAGGGGATAACGCAGGAAAGAACATGTGAGCAAAAGGCCAG |
| alpha | CAAAAGGCCAGGAACCGTAAAAAGGCCGCGTTGCTGGCGTTTTTCCAT |
| super | AGGCTCCGCCCCCCTGACGAGCATCACAAAAATCGACGCTCAAGTCAG |
| repressor- | AGGTGGCGAAACCCGACAGGACTATAAAGATACCAGGCGTTTCCCCCT |
| GFP | GGAAGCTCCCTCGTGCGCTCTCCTGTTCCGACCCTGCCGCTTACCGGAT |
| nucleic | ACCTGTCCGCCTTTCTCCCTTCGGGAAGCGTGGCGCTTTCTCATAGCTC |
| acid | ACGCTGTAGGTATCTCAGTTCGGTGTAGGTCGTTCGCTCCAAGCTGGG |
| sequence | CTGTGTGCACGAACCCCCCGTTCAGCCCGACCGCTGCGCCTTATCCGGT |
| | AACTATCGTCTTGAGTCCAACCCGGTAAGACACGACTTATCGCCACTG |
| | GCAGCAGCCACTGGTAACAGGATTAGCAGAGCGAGGTATGTAGGCGG |
| | TGCTACAGAGTTCTTGAAGTGGTGGCCTAACTACGGCTACACTAGAAG |
| | AACAGTATTTGGTATCTGCGCTCTGCTGAAGCCAGTTACCTTCGGAAA |
| | AAGAGTTGGTAGCTCTTGATCCGGCAAACAAACCACCGCTGGTAGCGG |
| | TGGTTTTTTTGTTTGCAAGCAGCAGATTACGCGCAGAAAAAAAGGATC |
| | TCAAGAAGATCCTTTGATCTTTTCTACGGGGTCTGACGCTCAGTGGAAC |
| | GAAAACTCACGTTAAGGGATTTTGGTCATGAGATTATCAAAAAGGATC |
| | TTCACCTAGATCCTTTTAAATTAAAAATGAAGTTTTAAATCAATCTAAA |
| | GTATATATGAGTAAACTTGGTCTGACAGTTACCAATGCTTAATCAGTG |
| | AGGCACCTATCTCAGCGATCTGTCTATTTCGTTCATCCATAGTTGCCTG |
| | ACTCCCCGTCGTGTAGATAACTACGATACGGGAGGGCTTACCATCTGG |
| | CCCCAGTGCTGCAATGATACCGCGAGACCCACGCTCACCGGCTCCAGA |
| | TTTATCAGCAATAAACCAGCCAGCCGGAAGGGCCGAGCGCAGAAGTG |
| | GTCCTGCAACTTTATCCGCCTCCATCCAGTCTATTAATTGTTGCCGGGA |
| | AGCTAGAGTAAGTAGTTCGCCAGTTAATAGTTTGCGCAACGTTGTTGC |
| | CATTGCTACAGGCATCGTGGTGTCACGCTCGTCGTTTGGTATGGCTTCA |
| | TTCAGCTCCGGTTCCCAACGATCAAGGCGAGTTACATGATCCCCCATG |
| | TTGTGCAAAAAAGCGGTTAGCTCCTTCGGTCCTCCGATCGTTGTCAGA |
| | AGTAAGTTGGCCGCAGTGTTATCACTCATGGTTATGGCAGCACTGCAT |
| | AATTCTCTTACTGTCATGCCATCCGTAAGATGCTTTTCTGTGACTGGTG |
| | AGTACTCAACCAAGTCATTCTGAGAATAGTGTATGCGGCGACCGAGTT |
| | GCTCTTGCCCGGCGTCAATACGGGATAATACCGCGCCACATAGCAGAA |
| | CTTTAAAAGTGCTCATCATTGGAAAACGTTCTTCGGGGCGAAAACTCT |
| | CAAGGATCTTACCGCTGTTGAGATCCAGTTCGATGTAACCCACTCGTG |
| | CACCCAACTGATCTTCAGCATCTTTTACTTTCACCAGCGTTTCTGGGTG |
| | AGCAAAAACAGGAAGGCAAAATGCCGCAAAAAAGGGAATAAGGGCG |
| | ACACGGAAATGTTGAATACTCATACTCTTCCTTTTTCAATATTATTGAA |
| | GCATTTATCAGGGTTATTGTCTCATGAGCGGATACATATTTGAATGTAT |
| | TTAGAAAAATAAACAAATAGGGGTTCCGCGCACATTTCCCCGAAAAGT |
| | GCCACCTAAATTGTAAGCGTTAATATTTTGTTAAAATTCGCGTTAAATT |
| | TTTGTTAAATCAGCTCATTTTTTAACCAATAGGCCGAAATCGGCAAAAT |
| | CCCTTATAAATCAAAAGAATAGACCGAGATAGGGTTGAGTGTTGTTCC |
| | AGTTTGGAACAAGAGTCCACTATTAAAGAACGTGGACTCCAACGTCAA |
| | AGGGCGAAAAACCGTCTATCAGGGCGATGGCCCACTACGTGAACCATC |
| | ACCCTAATCAAGTTTTTTGGGGTCGAGGTGCCGTAAAGCACTAAATCG |
| | GAACCCTAAAGGGAGCCCCCGATTTAGAGCTTGACGGGGAAAGCCGG |
| | CGAACGTGGCGAGAAAGGAAGGGAAGAAAGCGAAAGGAGCGGGCGC |
| | TAGGGCGCTGGCAAGTGTAGCGGTCACGCTGCGCGTAACCACCACACC |
| | CGCCGCGCTTAATGCGCCGCTACAGGGCGCGTCCCATTCGCCATTCAG |
| | GCTGCGCAACTGTTGGGAAGGGCGATCGGTGCGGGCCTCTTCGCTATT |
| | ACGCCAGCTGCGCGCTCGCTCGCTCACTGAGGCCGCCCGGGCAAAGCC |
| | CGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTT |
| | AATGATTAACCCGCCATGCTACTTATCTACGTAGCCATGCTCTAGGAA |
| | GATTTTCCTGTTTCGCGGGGGCCGGGGTGGAGGCGGAGAAGGAGGCG |
| | AGGAGGCAGCGGAGAAGGGGGCAGCCGGGAGGGGGGAGGGGACCGG |
| | AGAAGGAGGCGACGCGGGGGGGGGAAAGAGGAAGAGCCGGGGATGC |
| | TCGCGCTTAAAACCGAAAATAAACAACGTCCTCGGGCTGCGGCCTCGT |
| | GTCTGCCCGACCGCGCGGGGGCCTGGCCGGGACACACTTACCGGTCCG |
| | CCGCGGCGTGCAGGCCCTGGTCCCGGGTACACGCAGCGGCTCTTCTCT |
| | GCGCGCTGTTCCCAACCGGAGGAGCGCAGCGTACCGCTAAGGGTTCAA |
| | AACGCCCGCGCGCGTCCTGATTGGCGGGCCGGCCCATGGCTGGCCTCG |
| | CCGATCTTCCTATTTTCTTCTTTTTATAGATGGACTCGGAGCAAGGGAA |
| | ACCTCGCCGAGGGGCGTCAGGCATAGACGAAAGTTTCGGGACCCGAA |
| | AGTTAACCGGAGCGAAAGCCGATCGGCGCAGGAGACGTTGCCGGCCG |
| | GCTGCGAAAGGGCGCAGCTACTGCTAGGGGGCGGGCGGTGCGCACGC |
| | GTCCCGCGGCGCTGGCTGTCACCGCGATGGGTGGCGAGCGAAGCGAG |
| | CGGGATCCGGAGCGTGCCCTGCGCGGGAGGCAGCGGGACCCCGGATG |
| | CGGCAGGGCCCGCGCGCGCCTCCCCCTGGGCGCCTCTGGGAAGCTTTC |
| | CCGCGCGACTGGGGACCGCGCGCCTTGGGACTCGGGGGCGCGCCTGA |
| | GGGCGGAGATGGGCGTGGAGCAAAGATGGGCCGGGGGCGGCGCGTGG |
| | GTCTCGAGGTGCCTCAACGCCGAAGGGGCTGGGGGCGGCGCTTCTCAC |
| | CTCGCTTGTCACAGTGAGGCCGCCGCTGAGGGAGTACAGCAGCGGGA |
| | GCGCCACCATGTTCCAGGCGGCCGAGCGTCCCCAGGAGTGGGCCATG |
| | GAGGGCCCCCGCGACGGGCTGAAGAAGGAGCGGCTACTGGACGACCG |
| | CCACGACGCCGGCCTGGACGCCATGAAAGACGAGGAGTACGAGCAGA |
| | TGGTCAAGGAGCTGCAGGAGATCCGCCTCGAGCCGCAGGAGGTGCCG |
| | CGCGGCTCGGAGCCCTGGAAGCAGCAGCTCACCGAGGACGGGGACTC |
| | GTTCCTGCACTTGGCCATCATCCATGAAGAAAAGGCACTGACCATGGA |
| | AGTGATCCGCCAGGTGAAGGGAGACCTGGCCTTCCTCAACTTCCAGAA |
| | CAACCTGCAGCAGACTCCACTCCACTTGGCTGTGATCACCAACCAGCC |
| | AGAAATTGCTGAGGCACTTCTGGGAGCTGGCTGTGATCCTGAGCTCCG |
| | AGACTTTCGAGGAAATACCCCCCTACACCTTGCCTGTGAGCAGGGCTG |
| | CCTGGCCAGCGTGGGAGTCCTGACTCAGTCCTGCACCACCCCGCACCT |
| | CCACTCCATCCTGAAGGCTACCAACTACAATGGCCACACGTGTCTACA |
| | CTTAGCCTCTATCCATGGCTACCTGGGCATCGTGGAGCTTTTGGTGTCC |
| | TTGGGTGCTGATGTCAATGCTCAGGAGCCCTGTAATGGCCGGACTGCC |
| | CTTCACCTCGCAGTGGACCTGCAAAATCCTGACCTGGTGTCACTCCTGT |
| | TGAAGTGTGGGGCTGATGTCAACAGAGTTACCTACCAGGGCTATTCTC |
| | CCTACCAGCTCACCTGGGGCCGCCCAAGCACCCGGATACAGCAGCAGC |
| | TGGGCCAGCTGACACTAGAAAACCTTCAGATGCTGCCAGAGAGTGAG |
| | GATGAGGAGAGCTATGACACAGAGTCAGAGTTCACGGAGTTCACAGA |
| | GGACGAGCTGCCCTATGATGACTGTGTGTTTGGAGGCCAGCGTCTGAC |
| | GTTAGGATCCGGAGAGGGCAGAGGAAGTCTTCTAACATGCGGTGACGT |
| | GGAGGAGAATCCCGGCCCTATGGTGAGCAAGGGCGAGGAGCTGTTCA |
| | CCGGGGTGGTGCCCATCCTGGTCGAGCTGGACGGCGACGTAAACGGCC |
| | ACAAGTTCAGCGTGTCCGGCGAGGGCGAGGGCGATGCCACCTACGGC |
| | AAGCTGACCCTGAAGTTCATTTGCACCACCGGCAAGCTGCCCGTGCCC |
| | TGGCCCACCCTCGTGACCACCCTGACCTACGGCGTGCAGTGCTTCAGC |
| | CGCTACCCCGACCACATGAAGCAGCACGACTTCTTCAAGTCCGCCATG |
| | CCCGAAGGCTACGTCCAGGAGCGCACCATCTTCTTCAAGGACGACGGC |
| | AACTACAAGACCCGCGCCGAGGTGAAGTTCGAGGGCGACACCCTGGT |
| | GAACCGCATCGAGCTGAAGGGCATCGACTTCAAGGAGGACGGCAACA |
| | TCCTGGGGCACAAGCTGGAGTACAACTACAACAGCCACAACGTCTATA |
| | TCATGGCCGACAAGCAGAAGAACGGCATCAAGGTGAACTTCAAGATC |
| | CGCCACAACATCGAGGACGGCAGCGTGCAGCTCGCCGACCACTACCA |
| | GCAGAACACCCCCATCGGCGACGGCCCCGTGCTGCTGCCCGACAACCA |
| | CTACCTGAGCACCCAGTCCGCCCTGAGCAAAGACCCCAACGAGAAGC |
| | GCGATCACATGGTCCTGCTGGAGTTCGTGACCGCCGCCGGGATCACTC |
| | TCGGCATGGACGAGCTGTACAAGTAAAATCAACCTCTGGATTACAAAA |
| | TTTGTGAAAGATTGACTGGTATTCTTAACTATGTTGCTCCTTTTACGCT |
| | ATGTGGATACGCTGCTTTAATGCCTTTGTATCATGCTATTGCTTCCCGT |
| | ATGGCTTTCATTTTCTCCTCCTTGTATAAATCCTGGTTAGTTCTTGCCAC |
| | GGCGGAACTCATCGCCGCCTGCCTTGCCCGCTGCTGGACAGGGGCTCG |
| | GCTGTTGGGCACTGACAATTCCGTGGTGTTTATTTGTGAAATTTGTGAT |
| | GCTATTGCTTTATTTGTAACCATCTAGCTTTATTTGTGAAATTTGTGATG |
| | CTATTGCTTTATTTGTAACCATTATAAGCTGCAATAAACAAGTTTCTTC |
| | CTAGAGCATGGCTACGTAGATAAGTAGCATGGCGGGTTAATCATTAAC |
| | TACAAGGAACCCCTAGTGATGGAGTTGGCCACTCCCTCTCTGCGCGCT |
| | CGCTCGCTCACTGAGGCCGGGCGACCAAAGGTCGCCCGACGCCCGGGC |
| | TTTGCCCGGGCGGCCTCAGTGAGCGAGCGAGCGCGCAGCTG |
| 13 | CATTAATGAATCGGCCAACGCGCGGGGAGAGGCGGTTTGCGTATTGGG |
| AAV m- | CGCTCTTCCGCTTCCTCGCTCACTGACTCGCTGCGCTCGGTCGTTCGGC |
| CDKN2A- | TGCGGCGAGCGGTATCAGCTCACTCAAAGGCGGTAATACGGTTATCCA |
| OSK | CAGAATCAGGGGATAACGCAGGAAAGAACATGTGAGCAAAAGGCCAG |
| nucleic | CAAAAGGCCAGGAACCGTAAAAAGGCCGCGTTGCTGGCGTTTTTCCAT |
| acid | AGGCTCCGCCCCCCTGACGAGCATCACAAAAATCGACGCTCAAGTCAG |
| sequence | AGGTGGCGAAACCCGACAGGACTATAAAGATACCAGGCGTTTCCCCCT |
| | GGAAGCTCCCTCGTGCGCTCTCCTGTTCCGACCCTGCCGCTTACCGGAT |
| | ACCTGTCCGCCTTTCTCCCTTCGGGAAGCGTGGCGCTTTCTCATAGCTC |
| | ACGCTGTAGGTATCTCAGTTCGGTGTAGGTCGTTCGCTCCAAGCTGGG |
| | CTGTGTGCACGAACCCCCCGTTCAGCCCGACCGCTGCGCCTTATCCGGT |
| | AACTATCGTCTTGAGTCCAACCCGGTAAGACACGACTTATCGCCACTG |
| | GCAGCAGCCACTGGTAACAGGATTAGCAGAGCGAGGTATGTAGGCGG |
| | TGCTACAGAGTTCTTGAAGTGGTGGCCTAACTACGGCTACACTAGAAG |
| | AACAGTATTTGGTATCTGCGCTCTGCTGAAGCCAGTTACCTTCGGAAA |
| | AAGAGTTGGTAGCTCTTGATCCGGCAAACAAACCACCGCTGGTAGCGG |
| | TGGTTTTTTTGTTTGCAAGCAGCAGATTACGCGCAGAAAAAAAGGATC |
| | TCAAGAAGATCCTTTGATCTTTTCTACGGGGTCTGACGCTCAGTGGAAC |
| | GAAAACTCACGTTAAGGGATTTTGGTCATGAGATTATCAAAAAGGATC |
| | TTCACCTAGATCCTTTTAAATTAAAAATGAAGTTTTAAATCAATCTAAA |
| | GTATATATGAGTAAACTTGGTCTGACAGTTACCAATGCTTAATCAGTG |
| | AGGCACCTATCTCAGCGATCTGTCTATTTCGTTCATCCATAGTTGCCTG |
| | ACTCCCCGTCGTGTAGATAACTACGATACGGGAGGGCTTACCATCTGG |
| | CCCCAGTGCTGCAATGATACCGCGAGACCCACGCTCACCGGCTCCAGA |
| | TTTATCAGCAATAAACCAGCCAGCCGGAAGGGCCGAGCGCAGAAGTG |
| | GTCCTGCAACTTTATCCGCCTCCATCCAGTCTATTAATTGTTGCCGGGA |
| | AGCTAGAGTAAGTAGTTCGCCAGTTAATAGTTTGCGCAACGTTGTTGC |
| | CATTGCTACAGGCATCGTGGTGTCACGCTCGTCGTTTGGTATGGCTTCA |
| | TTCAGCTCCGGTTCCCAACGATCAAGGCGAGTTACATGATCCCCCATG |
| | TTGTGCAAAAAAGCGGTTAGCTCCTTCGGTCCTCCGATCGTTGTCAGA |
| | AGTAAGTTGGCCGCAGTGTTATCACTCATGGTTATGGCAGCACTGCAT |
| | AATTCTCTTACTGTCATGCCATCCGTAAGATGCTTTTCTGTGACTGGTG |
| | AGTACTCAACCAAGTCATTCTGAGAATAGTGTATGCGGCGACCGAGTT |
| | GCTCTTGCCCGGCGTCAATACGGGATAATACCGCGCCACATAGCAGAA |
| | CTTTAAAAGTGCTCATCATTGGAAAACGTTCTTCGGGGCGAAAACTCT |
| | CAAGGATCTTACCGCTGTTGAGATCCAGTTCGATGTAACCCACTCGTG |
| | CACCCAACTGATCTTCAGCATCTTTTACTTTCACCAGCGTTTCTGGGTG |
| | AGCAAAAACAGGAAGGCAAAATGCCGCAAAAAAGGGAATAAGGGCG |
| | ACACGGAAATGTTGAATACTCATACTCTTCCTTTTTCAATATTATTGAA |
| | GCATTTATCAGGGTTATTGTCTCATGAGCGGATACATATTTGAATGTAT |
| | TTAGAAAAATAAACAAATAGGGGTTCCGCGCACATTTCCCCGAAAAGT |
| | GCCACCTAAATTGTAAGCGTTAATATTTTGTTAAAATTCGCGTTAAATT |
| | TTTGTTAAATCAGCTCATTTTTTAACCAATAGGCCGAAATCGGCAAAAT |
| | CCCTTATAAATCAAAAGAATAGACCGAGATAGGGTTGAGTGTTGTTCC |
| | AGTTTGGAACAAGAGTCCACTATTAAAGAACGTGGACTCCAACGTCAA |
| | AGGGCGAAAAACCGTCTATCAGGGCGATGGCCCACTACGTGAACCATC |
| | ACCCTAATCAAGTTTTTTGGGGTCGAGGTGCCGTAAAGCACTAAATCG |
| | GAACCCTAAAGGGAGCCCCCGATTTAGAGCTTGACGGGGAAAGCCGG |
| | CGAACGTGGCGAGAAAGGAAGGGAAGAAAGCGAAAGGAGCGGGCGC |
| | TAGGGCGCTGGCAAGTGTAGCGGTCACGCTGCGCGTAACCACCACACC |
| | CGCCGCGCTTAATGCGCCGCTACAGGGCGCGTCCCATTCGCCATTCAG |
| | GCTGCGCAACTGTTGGGAAGGGCGATCGGTGCGGGCCTCTTCGCTATT |
| | ACGCCAGCTGCGCGCTCGCTCGCTCACTGAGGCCGCCCGGGCAAAGCC |
| | CGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTT |
| | AATGATTAACCCGCCATGCTACTTATCTACGTAGCCATGCTCTAGGAA |
| | GATTTTCCTGTTTCGCGGGGGCCGGGGTGGAGGCGGAGAAGGAGGCG |
| | AGGAGGCAGCGGAGAAGGGGGCAGCCGGGAGGGGGGAGGGGACCGG |
| | AGAAGGAGGCGACGCGGGGGGGGAAAGAGGAAGAGCCGGGGATGC |
| | TCGCGCTTAAAACCGAAAATAAACAACGTCCTCGGGCTGCGGCCTCGT |
| | GTCTGCCCGACCGCGCGGGGGCCTGGCCGGGACACACTTACCGGTCCG |
| | CCGCGGCGTGCAGGCCCTGGTCCCGGGTACACGCAGCGGCTCTTCTCT |
| | GCGCGCTGTTCCCAACCGGAGGAGCGCAGCGTACCGCTAAGGGTTCAA |
| | AACGCCCGCGCGCGTCCTGATTGGCGGGCCGGCCCATGGCTGGCCTCG |
| | CCGATCTTCCTATTTTCTTCTTTTTATAGATGGACTCGGAGCAAGGGAA |
| | ACCTCGCCGAGGGGCGTCAGGCATAGACGAAAGTTTCGGGACCCGAA |
| | AGTTAACCGGAGCGAAAGCCGATCGGCGCAGGAGACGTTGCCGGCCG |
| | GCTGCGAAAGGGCGCAGCTACTGCTAGGGGGCGGGCGGTGCGCACGC |
| | GTCCCGCGGCGCTGGCTGTCACCGCGATGGGTGGCGAGCGAAGCGAG |
| | CGGGATCCGGAGCGTGCCCTGCGCGGGAGGCAGCGGGACCCCGGATG |
| | CGGCAGGGCCCGCGCGCGCCTCCCCCTGGGCGCCTCTGGGAAGCTTTC |
| | CCGCGCGACTGGGGACCGCGCGCCTTGGGACTCGGGGGCGCGCCTGA |
| | GGGCGGAGATGGGCGTGGAGCAAAGATGGGCCGGGGGCGGCGCGTGG |
| | GTCTCGAGGTGCCTCAACGCCGAAGGGGCTGGGGGCGGCGCTTCTCAC |
| | CTCGCTTGTCACAGTGAGGCCGCCGCTGAGGGAGTACAGCAGCGGGA |
| | GCGCCACCATGGCTGGACACCTGGCTTCAGACTTCGCCTTCTCACCCCC |
| | ACCAGGTGGGGGTGATGGGTCAGCAGGGCTGGAGCCGGGCTGGGTGG |
| | ATCCTCGAACCTGGCTAAGCTTCCAAGGGCCTCCAGGTGGGCCTGGAA |
| | TCGGACCAGGCTCAGAGGTATTGGGGATCTCCCCATGTCCGCCCGCAT |
| | ACGAGTTCTGCGGAGGGATGGCATACTGTGGACCTCAGGTTGGACTGG |
| | GCCTAGTCCCCCAAGTTGGCGTGGAGACTTTGCAGCCTGAGGGCCAGG |
| | CAGGAGCACGAGTGGAAAGCAACTCAGAGGGAACCTCCTCTGAGCCC |
| | TGTGCCGACCGCCCCAATGCCGTGAAGTTGGAGAAGGTGGAACCAACT |
| | CCCGAGGAGTCCCAGGACATGAAAGCCCTGCAGAAGGAGCTAGAACA |
| | GTTTGCCAAGCTGCTGAAGCAGAAGAGGATCACCTTGGGGTACACCCA |
| | GGCCGACGTGGGGCTCACCCTGGGCGTTCTCTTTGGAAAGGTGTTCAG |
| | CCAGACCACCATCTGTCGCTTCGAGGCCTTGCAGCTCAGCCTTAAGAA |
| | CATGTGTAAGCTGCGGCCCCTGCTGGAGAAGTGGGTGGAGGAAGCCG |
| | ACAACAATGAGAACCTTCAGGAGATATGCAAATCGGAGACCCTGGTG |
| | CAGGCCCGGAAGAGAAAGCGAACTAGCATTGAGAACCGTGTGAGGTG |
| | GAGTCTGGAGACCATGTTTCTGAAGTGCCCGAAGCCCTCCCTACAGCA |
| | GATCACTCACATCGCCAATCAGCTTGGGCTAGAGAAGGATGTGGTTCG |
| | AGTATGGTTCTGTAACCGGCGCCAGAAGGGCAAAAGATCAAGTATTGA |
| | GTATTCCCAACGAGAAGAGTATGAGGCTACAGGGACACCTTTCCCAGG |
| | GGGGGCTGTATCCTTTCCTCTGCCCCCAGGTCCCCACTTTGGCACCCCA |
| | GGCTATGGAAGCCCCCACTTCACCACACTCTACTCAGTCCCTTTTCCTG |
| | AGGGCGAGGCCTTTCCCTCTGTTCCCGTCACTGCTCTGGGCTCTCCCAT |
| | GCATTCAAACGCTAGCGGCAGCGGCGCCACGAACTTCTCTCTGTTAAA |
| | GCAAGCAGGAGATGTTGAAGAAAACCCCGGGCCTGCATGCATGTATA |
| | ACATGATGGAGACGGAGCTGAAGCCGCCGGGCCCGCAGCAAGCTTCG |
| | GGGGGCGGCGGCGGAGGAGGCAACGCCACGGCGGCGGCGACCGGCG |
| | GCAACCAGAAGAACAGCCCGGACCGCGTCAAGAGGCCCATGAACGCC |
| | TTCATGGTATGGTCCCGGGGGCAGCGGCGTAAGATGGCCCAGGAGAA |
| | CCCCAAGATGCACAACTCGGAGATCAGCAAGCGCCTGGGCGCGGAGT |
| | GGAAACTTTTGTCCGAGACCGAGAAGCGGCCGTTCATCGACGAGGCCA |
| | AGCGGCTGCGCGCTCTGCACATGAAGGAGCACCCGGATTATAAATACC |
| | GGCCGCGGCGGAAAACCAAGACGCTCATGAAGAAGGATAAGTACACG |
| | CTTCCCGGAGGCTTGCTGGCCCCCGGCGGGAACAGCATGGCGAGCGGG |
| | GTTGGGGTGGGCGCCGGCCTGGGTGCGGGCGTGAACCAGCGCATGGA |
| | CAGCTACGCGCACATGAACGGCTGGAGCAACGGCAGCTACAGCATGA |
| | TGCAGGAGCAGCTGGGCTACCCGCAGCACCCGGGCCTCAACGCTCACG |
| | GCGCGGCACAGATGCAACCGATGCACCGCTACGACGTCAGCGCCCTGC |
| | AGTACAACTCCATGACCAGCTCGCAGACCTACATGAACGGCTCGCCCA |
| | CCTACAGCATGTCCTACTCGCAGCAGGGCACCCCCGGTATGGCGCTGG |
| | GCTCCATGGGCTCTGTGGTCAAGTCCGAGGCCAGCTCCAGCCCCCCCG |
| | TGGTTACCTCTTCCTCCCACTCCAGGGCGCCCTGCCAGGCCGGGGACCT |
| | CCGGGACATGATCAGCATGTACCTCCCCGGCGCCGAGGTGCCGGAGCC |
| | CGCTGCGCCCAGTAGACTGCACATGGCCCAGCACTACCAGAGCGGCCC |
| | GGTGCCCGGCACGGCCATTAACGGCACACTGCCCCTGTCGCACATGGC |
| | ATGCGGCTCCGGCGAGGGCAGGGGAAGTCTTCTAACATGCGGGGACG |
| | TGGAGGAAAATCCCGGCCCACTCGAGATGAGGCAGCCACCTGGCGAG |
| | TCTGACATGGCTGTCAGCGACGCTCTGCTCCCGTCCTTCTCCACGTTCG |
| | CGTCCGGCCCGGCGGGAAGGGAGAAGACACTGCGTCCAGCAGGTGCC |
| | CCGACTAACCGTTGGCGTGAGGAACTCTCTCACATGAAGCGACTTCCC |
| | CCACTTCCCGGCCGCCCCTACGACCTGGCGGCGACGGTGGCCACAGAC |
| | CTGGAGAGTGGCGGAGCTGGTGCAGCTTGCAGCAGTAACAACCCGGC |
| | CCTCCTAGCCCGGAGGGAGACCGAGGAGTTCAACGACCTCCTGGACCT |
| | AGACTTTATCCTTTCCAACTCGCTAACCCACCAGGAATCGGTGGCCGC |
| | CACCGTGACCACCTCGGCGTCAGCTTCATCCTCGTCTTCCCCAGCGAGC |
| | AGCGGCCCTGCCAGCGCGCCCTCCACCTGCAGCTTCAGCTATCCGATC |
| | CGGGCCGGGGGTGACCCGGGCGTGGCTGCCAGCAACACAGGTGGAGG |
| | GCTCCTCTACAGCCGAGAATCTGCGCCACCTCCCACGGCCCCCTTCAA |
| | CCTGGCGGACATCAATGACGTGAGCCCCTCGGGCGGCTTCGTGGCTGA |
| | GCTCCTGCGGCCGGAGTTGGACCCAGTATACATTCCGCCACAGCAGCC |
| | TCAGCCGCCAGGTGGCGGGCTGATGGGCAAGTTTGTGCTGAAGGCGTC |
| | TCTGACCACCCCTGGCAGCGAGTACAGCAGCCCTTCGGTCATCAGTGT |
| | TAGCAAAGGAAGCCCAGACGGCAGCCACCCCGTGGTAGTGGCGCCCT |
| | ACAGCGGTGGCCCGCCGCGCATGTGCCCCAAGATTAAGCAAGAGGCG |
| | GTCCCGTCCTGCACGGTCAGCCGGTCCCTAGAGGCCCATTTGAGCGCT |
| | GGACCCCAGCTCAGCAACGGCCACCGGCCCAACACACACGACTTCCCC |
| | CTGGGGCGGCAGCTCCCCACCAGGACTACCCCTACACTGAGTCCCGAG |
| | GAACTGCTGAACAGCAGGGACTGTCACCCTGGCCTGCCTCTTCCCCCA |
| | GGATTCCATCCCCATCCGGGGCCCAACTACCCTCCTTTCCTGCCAGACC |
| | AGATGCAGTCACAAGTCCCCTCTCTCCATTATCAAGAGCTCATGCCAC |
| | CGGGTTCCTGCCTGCCAGAGGAGCCCAAGCCAAAGAGGGGAAGAAGG |
| | TCGTGGCCCCGGAAAAGAACAGCCACCCACACTTGTGACTATGCAGGC |
| | TGTGGCAAAACCTATACCAAGAGTTCTCATCTCAAGGCACACCTGCGA |
| | ACTCACACAGGCGAGAAACCTTACCACTGTGACTGGGACGGCTGTGGG |
| | TGGAAATTCGCCCGCTCCGATGAACTGACCAGGCACTACCGCAAACAC |
| | ACAGGGCACCGGCCCTTTCAGTGCCAGAAGTGCGACAGGGCCTTTTCC |
| | AGGTCGGACCACCTTGCCTTACACATGAAGAGGCACTTTTAAAATCAA |
| | CCTCTGGATTACAAAATTTGTGAAAGATTGACTGGTATTCTTAACTATG |
| | TTGCTCCTTTTACGCTATGTGGATACGCTGCTTTAATGCCTTTGTATCAT |
| | GCTATTGCTTCCCGTATGGCTTTCATTTTCTCCTCCTTGTATAAATCCTG |
| | GTTAGTTCTTGCCACGGCGGAACTCATCGCCGCCTGCCTTGCCCGCTGC |
| | TGGACAGGGGCTCGGCTGTTGGGCACTGACAATTCCGTGGTGTTTATTT |
| | GTGAAATTTGTGATGCTATTGCTTTATTTGTAACCATCTAGCTTTATTTG |
| | TGAAATTTGTGATGCTATTGCTTTATTTGTAACCATTATAAGCTGCAAT |
| | AAACAAGTTTCTTCCTAGAGCATGGCTACGTAGATAAGTAGCATGGCG |
| | GGTTAATCATTAACTACAAGGAACCCCTAGTGATGGAGTTGGCCACTC |
| | CCTCTCTGCGCGCTCGCTCGCTCACTGAGGCCGGGCGACCAAAGGTCG |
| | CCCGACGCCCGGGCTTTGCCCGGGCGGCCTCAGTGAGCGAGCGAGCGC |
| | GCAGCTG |
| 14 | CATTAATGAATCGGCCAACGCGCGGGGAGAGGCGGTTTGCGTATTGGG |
| AAV m- | CGCTCTTCCGCTTCCTCGCTCACTGACTCGCTGCGCTCGGTCGTTCGGC |
| CDKN2A | TGCGGCGAGCGGTATCAGCTCACTCAAAGGCGGTAATACGGTTATCCA |
| CBRLuc- | CAGAATCAGGGGATAACGCAGGAAAGAACATGTGAGCAAAAGGCCAG |
| GFP | CAAAAGGCCAGGAACCGTAAAAAGGCCGCGTTGCTGGCGTTTTTCCAT |
| nucleic | AGGCTCCGCCCCCCTGACGAGCATCACAAAAATCGACGCTCAAGTCAG |
| acid | AGGTGGCGAAACCCGACAGGACTATAAAGATACCAGGCGTTTCCCCCT |
| sequence | GGAAGCTCCCTCGTGCGCTCTCCTGTTCCGACCCTGCCGCTTACCGGAT |
| | ACCTGTCCGCCTTTCTCCCTTCGGGAAGCGTGGCGCTTTCTCATAGCTC |
| | ACGCTGTAGGTATCTCAGTTCGGTGTAGGTCGTTCGCTCCAAGCTGGG |
| | CTGTGTGCACGAACCCCCCGTTCAGCCCGACCGCTGCGCCTTATCCGGT |
| | AACTATCGTCTTGAGTCCAACCCGGTAAGACACGACTTATCGCCACTG |
| | GCAGCAGCCACTGGTAACAGGATTAGCAGAGCGAGGTATGTAGGCGG |
| | TGCTACAGAGTTCTTGAAGTGGTGGCCTAACTACGGCTACACTAGAAG |
| | AACAGTATTTGGTATCTGCGCTCTGCTGAAGCCAGTTACCTTCGGAAA |
| | AAGAGTTGGTAGCTCTTGATCCGGCAAACAAACCACCGCTGGTAGCGG |
| | TGGTTTTTTTGTTTGCAAGCAGCAGATTACGCGCAGAAAAAAAGGATC |
| | TCAAGAAGATCCTTTGATCTTTTCTACGGGGTCTGACGCTCAGTGGAAC |
| | GAAAACTCACGTTAAGGGATTTTGGTCATGAGATTATCAAAAAGGATC |
| | TTCACCTAGATCCTTTTAAATTAAAAATGAAGTTTTAAATCAATCTAAA |
| | GTATATATGAGTAAACTTGGTCTGACAGTTACCAATGCTTAATCAGTG |
| | AGGCACCTATCTCAGCGATCTGTCTATTTCGTTCATCCATAGTTGCCTG |
| | ACTCCCCGTCGTGTAGATAACTACGATACGGGAGGGCTTACCATCTGG |
| | CCCCAGTGCTGCAATGATACCGCGAGACCCACGCTCACCGGCTCCAGA |
| | TTTATCAGCAATAAACCAGCCAGCCGGAAGGGCCGAGCGCAGAAGTG |
| | GTCCTGCAACTTTATCCGCCTCCATCCAGTCTATTAATTGTTGCCGGGA |
| | AGCTAGAGTAAGTAGTTCGCCAGTTAATAGTTTGCGCAACGTTGTTGC |
| | CATTGCTACAGGCATCGTGGTGTCACGCTCGTCGTTTGGTATGGCTTCA |
| | TTCAGCTCCGGTTCCCAACGATCAAGGCGAGTTACATGATCCCCCATG |
| | TTGTGCAAAAAAGCGGTTAGCTCCTTCGGTCCTCCGATCGTTGTCAGA |
| | AGTAAGTTGGCCGCAGTGTTATCACTCATGGTTATGGCAGCACTGCAT |
| | AATTCTCTTACTGTCATGCCATCCGTAAGATGCTTTTCTGTGACTGGTG |
| | AGTACTCAACCAAGTCATTCTGAGAATAGTGTATGCGGCGACCGAGTT |
| | GCTCTTGCCCGGCGTCAATACGGGATAATACCGCGCCACATAGCAGAA |
| | CTTTAAAAGTGCTCATCATTGGAAAACGTTCTTCGGGGCGAAAACTCT |
| | CAAGGATCTTACCGCTGTTGAGATCCAGTTCGATGTAACCCACTCGTG |
| | CACCCAACTGATCTTCAGCATCTTTTACTTTCACCAGCGTTTCTGGGTG |
| | AGCAAAAACAGGAAGGCAAAATGCCGCAAAAAAGGGAATAAGGGCG |
| | ACACGGAAATGTTGAATACTCATACTCTTCCTTTTTCAATATTATTGAA |
| | GCATTTATCAGGGTTATTGTCTCATGAGCGGATACATATTTGAATGTAT |
| | TTAGAAAAATAAACAAATAGGGGTTCCGCGCACATTTCCCCGAAAAGT |
| | GCCACCTAAATTGTAAGCGTTAATATTTTGTTAAAATTCGCGTTAAATT |
| | TTTGTTAAATCAGCTCATTTTTTAACCAATAGGCCGAAATCGGCAAAAT |
| | CCCTTATAAATCAAAAGAATAGACCGAGATAGGGTTGAGTGTTGTTCC |
| | AGTTTGGAACAAGAGTCCACTATTAAAGAACGTGGACTCCAACGTCAA |
| | AGGGCGAAAAACCGTCTATCAGGGCGATGGCCCACTACGTGAACCATC |
| | ACCCTAATCAAGTTTTTTGGGGTCGAGGTGCCGTAAAGCACTAAATCG |
| | GAACCCTAAAGGGAGCCCCCGATTTAGAGCTTGACGGGGAAAGCCGG |
| | CGAACGTGGCGAGAAAGGAAGGGAAGAAAGCGAAAGGAGCGGGCGC |
| | TAGGGCGCTGGCAAGTGTAGCGGTCACGCTGCGCGTAACCACCACACC |
| | CGCCGCGCTTAATGCGCCGCTACAGGGCGCGTCCCATTCGCCATTCAG |
| | GCTGCGCAACTGTTGGGAAGGGCGATCGGTGCGGGCCTCTTCGCTATT |
| | ACGCCAGCTGCGCGCTCGCTCGCTCACTGAGGCCGCCCGGGCAAAGCC |
| | CGGGCGTCGGGCGACCTTTGGTCGCCCGGCCTCAGTGAGCGAGCGAGC |
| | GCGCAGAGAGGGAGTGGCCAACTCCATCACTAGGGGTTCCTTGTAGTT |
| | AATGATTAACCCGCCATGCTACTTATCTACGTAGCCATGCTCTAGGAA |
| | GATTTTCCTGTTTCGCGGGGGCCGGGGTGGAGGCGGAGAAGGAGGCG |
| | AGGAGGCAGCGGAGAAGGGGGCAGCCGGGAGGGGGGAGGGGACCGG |
| | AGAAGGAGGCGACGCGGGGGGGGGAAAGAGGAAGAGCCGGGGATGC |
| | TCGCGCTTAAAACCGAAAATAAACAACGTCCTCGGGCTGCGGCCTCGT |
| | GTCTGCCCGACCGCGCGGGGGCCTGGCCGGGACACACTTACCGGTCCG |
| | CCGCGGCGTGCAGGCCCTGGTCCCGGGTACACGCAGCGGCTCTTCTCT |
| | GCGCGCTGTTCCCAACCGGAGGAGCGCAGCGTACCGCTAAGGGTTCAA |
| | AACGCCCGCGCGCGTCCTGATTGGCGGGCCGGCCCATGGCTGGCCTCG |
| | CCGATCTTCCTATTTTCTTCTTTTTATAGATGGACTCGGAGCAAGGGAA |
| | ACCTCGCCGAGGGGCGTCAGGCATAGACGAAAGTTTCGGGACCCGAA |
| | AGTTAACCGGAGCGAAAGCCGATCGGCGCAGGAGACGTTGCCGGCCG |
| | GCTGCGAAAGGGCGCAGCTACTGCTAGGGGGGGGGGGGTGCGCACGC |
| | GTCCCGCGGCGCTGGCTGTCACCGCGATGGGTGGCGAGCGAAGCGAG |
| | CGGGATCCGGAGCGTGCCCTGCGCGGGAGGCAGCGGGACCCCGGATG |
| | CGGCAGGGCCCGCGCGCGCCTCCCCCTGGGCGCCTCTGGGAAGCTTTC |
| | CCGCGCGACTGGGGACCGCGCGCCTTGGGACTCGGGGGCGCGCCTGA |
| | GGGCGGAGATGGGCGTGGAGCAAAGATGGGCCGGGGGCGGCGCGTGG |
| | GTCTCGAGGTGCCTCAACGCCGAAGGGGCTGGGGGCGGCGCTTCTCAC |
| | CTCGCTTGTCACAGTGAGGCCGCCGCTGAGGGAGTACAGCAGCGGGA |
| | GCGCCACCATGGATGGTAAAGCGTGAGAAAAATGTCATCTATGGCCCT |
| | GAGCCTCTCCATCCTTTGGAGGATTTGACTGCCGGCGAAATGCTGTTTC |
| | GTGCTCTCCGCAAGCACTCTCATTTGCCTCAAGCCTTGGTCGATGTGGT |
| | CGGCGATGAATCTTTGAGCTACAAGGAGTTTTTTGAGGCAACCGTCTT |
| | GCTGGCTCAGTCCCTCCACAATTGTGGCTACAAGATGAACGACGTCGT |
| | TAGTATCTGTGCTGAAAACAATACCCGTTTCTTCATTCCAGTCATCGCC |
| | GCATGGTATATCGGTATGATCGTGGCTCCAGTCAACGAGAGCTACATT |
| | CCCGACGAACTGTGTAAAGTCATGGGTATCTCTAAGCCACAGATTGTC |
| | TTCACCACTAAGAATATTCTGAACAAAGTCCTGGAAGTCCAAAGCCGC |
| | ACCAACTTTATTAAGCGTATCATCATCTTGGACACTGTGGAGAATATTC |
| | ACGGTTGCGAATCTTTGCCTAATTTCATCTCTCGCTATTCAGACGGCAA |
| | CATCGCAAACTTTAAACCACTCCACTTCGACCCTGTGGAACAAGTTGC |
| | AGCCATTCTGTGTAGCAGCGGTACTACTGGACTCCCAAAGGGAGTCAT |
| | GCAGACCCATCAAAACATTTGCGTGCGTCTGATCCATGCTCTCGATCC |
| | ACGCTACGGCACTCAGCTGATTCCTGGTGTCACCGTCTTGGTCTACTTG |
| | CCTTTCTTCCATGCTTTCGGCTTTCATATTACTTTGGGTTACTTTATGGT |
| | CGGTCTCCGCGTGATTATGTTCCGCCGTTTTGATCAGGAGGCTTTCTTG |
| | AAAGCCATCCAAGATTATGAAGTCCGCAGTGTCATCAACGTGCCTAGC |
| | GTGATCCTGTTTTTGTCTAAGAGCCCACTCGTGGACAAGTACGACTTGT |
| | CTTCACTGCGTGAATTGTGTTGCGGTGCCGCTCCACTGGCTAAGGAGG |
| | TCGCTGAAGTGGCCGCCAAACGCTTGAATCTTCCAGGGATTCGTTGTG |
| | GCTTCGGCCTCACCGAATCTACCAGTGCGATTATCCAGACTCTCGGGG |
| | ATGAGTTTAAGAGCGGCTCTTTGGGCCGTGTCACTCCACTCATGGCTGC |
| | TAAGATCGCTGATCGCGAAACTGGTAAGGCTTTGGGCCCGAACCAAGT |
| | GGGCGAGCTGTGTATCAAAGGCCCTATGGTGAGCAAGGGTTATGTCAA |
| | TAACGTTGAAGCTACCAAGGAGGCCATCGACGACGACGGCTGGTTGCA |
| | TTCTGGTGATTTTGGATATTACGACGAAGATGAGCATTTTTACGTCGTG |
| | GATCGTTACAAGGAGCTGATCAAATACAAGGGTAGCCAGGTTGCTCCA |
| | GCTGAGTTGGAGGAGATTCTGTTGAAAAATCCATGCATTCGCGATGTC |
| | GCTGTGGTCGGCATTCCTGATCTGGAGGCCGGCGAACTGCCTTCTGCTT |
| | TCGTTGTCAAGCAGCCTGGTACAGAAATTACCGCCAAAGAAGTGTATG |
| | ATTACCTGGCTGAACGTGTGAGCCATACTAAGTACTTGCGTGGCGGCG |
| | TGCGTTTTGTTGACTCCATCCCTCGTAACGTAACAGGCAAAATTACCCG |
| | CAAGGAGCTGTTGAAACAATTGTTGGTGAAGGCCGGCGGTGGATCCGG |
| | AGAGGGCAGAGGAAGTCTTCTAACATGCGGTGACGTGGAGGAGAATC |
| | CCGGCCCTATGGTGAGCAAGGGCGAGGAGCTGTTCACCGGGGTGGTGC |
| | CCATCCTGGTCGAGCTGGACGGCGACGTAAACGGCCACAAGTTCAGCG |
| | TGTCCGGCGAGGGCGAGGGCGATGCCACCTACGGCAAGCTGACCCTG |
| | AAGTTCATTTGCACCACCGGCAAGCTGCCCGTGCCCTGGCCCACCCTC |
| | GTGACCACCCTGACCTACGGCGTGCAGTGCTTCAGCCGCTACCCCGAC |
| | CACATGAAGCAGCACGACTTCTTCAAGTCCGCCATGCCCGAAGGCTAC |
| | GTCCAGGAGCGCACCATCTTCTTCAAGGACGACGGCAACTACAAGACC |
| | CGCGCCGAGGTGAAGTTCGAGGGCGACACCCTGGTGAACCGCATCGA |
| | GCTGAAGGGCATCGACTTCAAGGAGGACGGCAACATCCTGGGGCACA |
| | AGCTGGAGTACAACTACAACAGCCACAACGTCTATATCATGGCCGACA |
| | AGCAGAAGAACGGCATCAAGGTGAACTTCAAGATCCGCCACAACATC |
| | GAGGACGGCAGCGTGCAGCTCGCCGACCACTACCAGCAGAACACCCC |
| | CATCGGCGACGGCCCCGTGCTGCTGCCCGACAACCACTACCTGAGCAC |
| | CCAGTCCGCCCTGAGCAAAGACCCCAACGAGAAGCGCGATCACATGG |
| | TCCTGCTGGAGTTCGTGACCGCCGCCGGGATCACTCTCGGCATGGACG |
| | AGCTGTACAAGTAAAATCAACCTCTGGATTACAAAATTTGTGAAAGAT |
| | TGACTGGTATTCTTAACTATGTTGCTCCTTTTACGCTATGTGGATACGC |
| | TGCTTTAATGCCTTTGTATCATGCTATTGCTTCCCGTATGGCTTTCATTT |
| | TCTCCTCCTTGTATAAATCCTGGTTAGTTCTTGCCACGGCGGAACTCAT |
| | CGCCGCCTGCCTTGCCCGCTGCTGGACAGGGGCTCGGCTGTTGGGCAC |
| | TGACAATTCCGTGGTGTTTATTTGTGAAATTTGTGATGCTATTGCTTTA |
| | TTTGTAACCATCTAGCTTTATTTGTGAAATTTGTGATGCTATTGCTTTAT |
| | TTGTAACCATTATAAGCTGCAATAAACAAGTTTCTTCCTAGAGCATGG |
| | CTACGTAGATAAGTAGCATGGCGGGTTAATCATTAACTACAAGGAACC |
| | CCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGC |
| | GGCCTCAGTGAGCGAGCGAGCGCGCAGCTGAGTGAGGCCGCCGCTGA |
| | GGGAGTACAGCAGCGGGAGCGCCACC |
| 15 | GGAACCCCTAGTGATGGAGTT |
| AAV | |
| ITR-F | |
| primer | |
| 16 | CGGCCTCAGTGAGCGA |
| AAV | |
| ITR-R | |
| primer | |
| 17 | GTGTGCATGACGTGCGGG |
| Cdkn2a-F | |
| primer | |
| 18 | GTTGCCCATCATCATCACCTGAA |
| Cdkn2a-R | |
| primer | |
| 19 | GCCTGCAGCCACTGCGACAA |
| Ctcf-F | |
| primer | |
| 20 | CCATTGTGTTCCGGCGGGTGA |
| Ctcf-R | |
| primer | |
| 21 | AGGAGAAGACCAGCCCGTGT |
| Il1a-F | |
| primer | |
| 22 | TGCCAGGTGCACCCGACTTT |
| Il1a-R | |
| primer | |
| 23 | AAAGACGGCACACCCACCCT |
| Il1b-F | |
| primer | |
| 24 | TGTGCTCTGCTTGTGAGGTGCT |
| Il1b-R | |
| primer | |
| 25 | CAGCAGATCACTCACATCGC |
| Oct4-F | |
| primer | |
| 26 | CGCCGGTTACAGAACCATAC |
| Oct4-R | |
| primer | |
| 27 | CCTGGGCGCGGAGTGGAAAC |
| Sox2-F | |
| primer | |
| 28 | CCGCCGCGGCCGGTATTTAT |
| Sox2-R | |
| primer | |
| 29 | gggcttgagagctgggttctatt |
| CTCF-F | |
| primer | |
| 30 | cttcgactgcatcaccttccatt |
| CTCF-R | |
| primer | |
| 31 | gtgaacatcatggatcagaacaaca |
| TBP-F | |
| primer | |
| 32 | aagatagggattccgggagtcat |
| TBP-R | |
| primer | |
| 33 | acattcatgtgggcatttcttg |
| CDKN2A- | |
| F primer | |
| 34 | aatgcttgtcatgaagtcgacag |
| CDKN2A- | |
| R primer | |
| 35 | CCAGTGCTGCTGAAGGAGATGCC |
| IL1A-F | |
| primer | |
| 36 | CCCTGCCAAGCACACCCAGT |
| IL1A-R | |
| primer | |
| 37 | GCTCTCCACCTCCAGGGACAGG |
| IL1B-F | |
| primer | |
| 38 | TGAGGCCCAAGGCCACAGGT |
| IL1B-R | |
| primer | |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A polynucleotide comprising at least one CDKN2A promoter operably linked to a nucleic acid sequence encoding an Oct4 protein, and/or a nucleic acid sequence encoding a Sox2 protein, and/or a nucleic acid sequence encoding a Klf4 protein.

2. The polynucleotide of claim 1, wherein the CDKN2A promoter is operably linked to a nucleic acid sequence encoding an Oct4 protein, a Sox2 protein, and a Klf4 protein.

3. The polynucleotide of claim 1 or 2, wherein the CDKN2A promoter is a human or murine CDKN2A promoter.

4. The polynucleotide of any one of claims 1-3, wherein the human CDKN2A promoter has a nucleotide sequence at least 85%, 86%, 87%, 88%, 89%, 90%, 91%, 92%, 93%, 94%, 95%, 96%, 97%, 98%, 99%, or 100% identical to SEQ ID NO: 1.

5. The polynucleotide of any one of claims 1-3, wherein the murine CDKN2A promoter has a nucleotide sequence at least 85%, 86%, 87%, 88%, 89%, 90%, 91%, 92%, 93%, 94%, 95%, 96%, 97%, 98%, 99%, or 100% identical to SEQ ID NO: 2.

6. The polynucleotide of any one of claims 1-5, wherein polynucleotide encodes a human Oct4 protein, and/or a human Sox2 protein, and/or a human Klf4 protein.

7. The polynucleotide of any one of claims 1-6, wherein the nucleic acid sequence encoding Oct4 comprises SEQ ID NO: 3, the nucleic acid sequence encoding Sox2 comprises SEQ ID NO: 4, and the nucleic acid encoding Klf4 comprises SEQ ID NO: 5.

8. The polynucleotide of any one of claims 1-7, further comprising at least one Woodchuck Hepatitis Virus Posttranscriptional Regulatory Element (WPRE) sequence and at least one polyadenylation signal sequence.

9. The polynucleotide of claim 8, wherein the at least one polyadenylation signal sequence is an SV40 polyadenylation signal sequence, a human growth hormone polyadenylation signal sequence, or a bovine growth hormone polyadenylation signal sequence.

10. The polynucleotide of any one of claims 1-9, wherein the polynucleotide further comprises at least one internal ribosome entry site (IRES).

11. The polynucleotide of any one of claims 1-10, wherein the polynucleotide further comprises at least one proteolytic cleavage site.

12. The polynucleotide of claim 11, wherein the at least one proteolytic cleavage site is a self-processing cleavage site or a furin cleavage site.

13. The polynucleotide of claim 11 or 12, wherein the self-processing cleavage site is a P2A, E2A, F2A, or T2A peptide.

14. The polynucleotide of claim 12, wherein the furin cleavage site comprises the consensus sequence RXK(R)R of SEQ ID NO: 6.

15. The polynucleotide of any one of claims 1-14, wherein the polynucleotide further comprises a second CDKN2A promoter.

16. The polynucleotide of claim 15, wherein the first and the second promoter initiate transcription in the same direction.

17. The polynucleotide of claim 15, wherein the first and the second promoter initiate transcription in different directions.

18. A polynucleotide comprising the nucleic acid sequence of SEQ ID NO: 10.

19. A polynucleotide comprising a CDKN2A promoter operably linked to a nucleic acid sequence encoding a dominant-negative nuclear factor kappa BIA (dnNKκBIA) protein.

20. The polynucleotide of claim 19, wherein the CDKN2A promoter is a human or murine CDKN2A promoter.

21. The polynucleotide of claim 19 or 20, wherein the human CDKN2A promoter has a nucleotide sequence at least 85%, 86%, 87%, 88%, 89%, 90%, 91%, 92%, 93%, 94%, 95%, 96%, 97%, 98%, 99%, or 100% identical to SEQ ID NO: 1.

22. The polynucleotide of claim 19 or 20, wherein the murine CDKN2A promoter has a nucleotide sequence at least 85%, 86%, 87%, 88%, 89%, 90%, 91%, 92%, 93%, 94%, 95%, 96%, 97%, 98%, 99%, or 100% identical to SEQ ID NO: 2.

23. The polynucleotide of any one of claims 19-22, wherein the nucleic acid sequence encodes a human dnNKκBIA protein.

24. The polynucleotide of any one of claims 19-22, wherein the nucleic acid sequence encoding the dnNKκBIA protein comprises SEQ ID NO: 7.

25. The polynucleotide of any one of claims 19-22, wherein the polynucleotide further comprises a WPRE sequence and a polyadenylation signal sequence.

26. The polynucleotide of claim 25, wherein the polyadenylation signal sequence is an SV40 polyadenylation signal sequence, a human growth hormone polyadenylation signal sequence, or a bovine growth hormone polyadenylation signal sequence.

27. The polynucleotide of any one of claims 19-26, wherein the polynucleotide further comprises an IRES.

28. The polynucleotide of any one of claims 19-27, wherein the polynucleotide further comprises a proteolytic cleavage site.

29. The polynucleotide of claim 28, wherein the proteolytic cleavage site is selected from the group consisting of a self-processing cleavage site and a furin cleavage site.

30. The polynucleotide of claim 28 or 29, wherein the self-processing cleavage site is a P2A, E2A, F2A, or T2A peptide.

31. The polynucleotide of claim 29, wherein the furin cleavage site comprise the consensus sequence RXK(R)R of SEQ ID NO: 6.

32. The polynucleotide of any one of claims 19-31, wherein the polynucleotide further comprises a reporter gene.

33. A polynucleotide comprising the nucleic acid sequence of SEQ ID NO: 11.

34. A polynucleotide comprising the nucleic acid sequence of SEQ ID NO: 12.

35. A vector comprising the polynucleotide of any one of claims 1-18.

36. A vector comprising the polynucleotide of any one of claims 19-34.

37. The vector of claim 35 or 36, wherein the vector is a viral vector, a non-viral vector, or a polymer.

38. The vector of claim 37, wherein the viral vector is an adeno-associated viral (AAV) vector, an adenoviral vector, a lentiviral vector, or a retroviral vector.

39. The vector of claim 38, wherein the AAV vector is a AAV1, AAV2, AAV3, AAV4, AAV5, AAV6, AAV7, AAV8, AAVRH8, AAVrh9, AAV9, AAVrh10, AAV10, AAVRH10, AAV11, AAV12, or AAV-DJ vector.

40. The vector of claim 38 or 39, wherein the AAV vector is an AAV-DJ vector.

41. The vector of claim 37, wherein the non-viral vector is a plasmid.

42. The vector of claim 37, wherein the polymer is a cationic polymer comprising a polyethyleneimine (PEI) backbone linked to a lipid or a polyethylene glycol (PEG).

43. A cell comprising the polynucleotide of any one of claims 1-34 or the vector of any one of claims 35-42.

44. The cell of claim 43, wherein the cell is a bacterial cell, an insect cell, or an animal cell.

45. The cell of claim 44, wherein the animal cell is a mammalian cell.

46. A composition comprising the polynucleotide of any one of claims 1-34, or the vector of claims 35-42, and a carrier.

47. A recombinant adeno-associated vector (rAAV) virus comprising the polynucleotide of any one of claims 1-34 and an AAV capsid protein.

48. The rAAV of claim 47, comprising an AAV-DJ capsid.

49. A pharmaceutical composition comprising the polynucleotide of any one of claims 1-34, the vector of any one of claims 35-42, the cell of any one of claims 43-45, the composition of claim 46, or the rAAV of claim 47 or 48, and a pharmaceutically acceptable carrier.

50. A kit comprising the polynucleotide of any one of claims 1-34, the vector of any one of claims 35-42, the cell of any one of claims 43-45, the composition of claim 46, or the rAAV of claim 47 or 48.

51. A method for making a rAAV comprising:
(i) providing a cell with adeno-associated virus (AAV) rep genes, AAV cap genes, a nucleic acid comprising the polynucleotide of any one of claims 1-34, the vector of any one of claims 35-42, and at least one AAV serotype 2 inverted terminal repeat,
(ii) providing helper functions for generating a productive AAV infection;
(ii) allowing assembly of the AAV; and
(iii) collecting the rAAV.

52. The method of claim 51, wherein the AAV rep genes and AAV cap genes are provided by a plasmid.

53. The method of claim 51, wherein the AAV rep genes and AAV cap genes are provided by an adenovirus vector.

54. The method of claim 51, wherein the AAV rep genes and AAV cap genes are stably integrated into the genome of the cell.

55. The method of any one of claims 51-54, wherein the helper functions are provided by a plasmid.

56. The method of any one of claims 51-54, wherein the helper functions are provided by an adenovirus vector.

57. A rAAV produced by the method of any one of claims 51-56.

58. A method of reprogramming a senescent cell to a non-senescent stage phenotype, the method comprising contacting a senescent cell with the polynucleotide of any one of claims 1-34, the vector of any one of claims 35-42, the cell of any one of claims 43-45, the composition of claim 46, or the rAAV of claim 47 or 48, or the pharmaceutical composition of claim 49 in an amount effective to partially reprogram the senescent cell to a non-senescent stage phenotype.

59. A method for reprogramming senescent cells in a subject to a non-senescent stage phenotype, the method comprising:
(i) administering to the subject in need thereof a therapeutically effective amount of the polynucleotide of any one of claims 1-34, the vector of any one of claims 35-42, the cell of any one of claims 43-45, the composition of claim 46, the rAAV of claim 47 or 48, or the pharmaceutical composition of claim 49, and
(ii) partially reprogramming senescent cells of the subject to a non-senescent stage phenotype.

60. The method of claim 59, wherein the administering is by systemic administration.

61. A method for partially or fully reverting immune senescence in a subject, the method comprising:
administering to the subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to partially or fully revert immune senescence in the subject.

62. The method of claim 61, wherein the immune senescence is characterized by a decrease in a homing ability of a hematopoietic stem cell (HSC) and the amount effective to partially or fully revert immune senescence in the subject is an amount that is effective to increase the homing ability of the HSC to a level of HSC homing ability found in a HSC of a non-senescent subject.

63. The method of claim 61, wherein the immune senescence is characterized by a decrease in per-cell repopulating self-renewal activity of a HSC and the amount effective to partially or fully revert immune senescence in the subject is an amount effective to increase the per-cell repopulating self-renewal activity of the HSC to a level of HSC per-cell repopulating self-renewal activity found in a HSC of a non-senescent subject.

64. The method of claim 61, wherein the immune senescence is characterized by a bias towards myeloid differentiation of a HSC and the amount effective to partially or fully revert immune senescence in the subject is an amount effective to reduce the bias towards myeloid differentiation of the HSC to a level of bias towards myeloid differentiation found in a HSC of a non-senescent subject.

65. The method of claim 61, wherein the immune senescence is characterized by an increase in apoptosis of a HSC upon stress stimuli and the amount effective to partially or fully revert immune senescence in the subject is an amount effective to reduce apoptosis of the HSC upon stress stimuli to a level of apoptosis upon stress stimuli found in a HSC of a non-senescent subject.

66. The method of claim 61, wherein the administering is by systemic administration.

67. A method for reducing the number of senescent cells in a subject, the method comprising: administering to the subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to reduce the number of senescent cells in the subject.

68. The method of claim 67, wherein the administering is by systemic administration.

69. A method for extending organismal lifespan of a subject, the method comprising: administering to the subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to extend organismal lifespan of the subject.

70. The method of claim 69, wherein the administering is by systemic administration.

71. A method for reducing progressive body weight loss in a senescent subject, the method comprising: administering to the senescent subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to reduce progressive body weight loss in the senescent subject.

72. The method of claim 71, wherein the administering is by systemic administration.

73. A method for increasing physical fitness in a senescent subject, the method comprising: administering to the senescent subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to increase physical fitness in the senescent subject.

74. The method of claim 73, wherein the administering is by systemic administration.

75. A method for reducing an inflammatory response in a subject, the method comprising: administering to the subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to reduce an inflammatory response in the subject.

76. The method of claim 75, wherein the administering is by systemic administration.

77. A method for reducing replicative senescence in a subject, the method comprising: administering to the subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to reduce replicative senescence in the subject.

78. The method of claim 77, wherein the administering is by systemic administration.

79. A method for reducing DNA damage induced senescence in a subject, the method comprising: administering to the subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to reduce DNA damage induced senescence in the subject.

80. The method of claim 79, wherein the administering is by systemic administration.

81. A method of reducing oncogene induced senescence in a subject, the method comprising: administering to the subject in need thereof the polynucleotide of any one of claims 1-34, the vector of any one of claims 35-42, the cell of any one of claims 43-45, the composition of claim 46, the rAAV of claim 47 or 48, or the pharmaceutical composition of claim 49 in an amount effective to reduce oncogene induced senescence in the subject.

82. The method of claim 81, wherein the administering is by systemic administration.

83. A method for inhibiting Senescence-Associated Secretory Phenotype (SASP) activity in a cell, comprising contacting the cell with the polynucleotide of any one of claims 19-32, the vector of any one of claims 36-42, the cell of any one of claims 43-45, the composition of claim 46, the rAAV of claim 47 or 48, or the pharmaceutical composition of claim 49, in an amount effective to inhibit SASP activity in the cell.

84. A method for rejuvenating a senescent cell to a non-senescent stage phenotype, the method comprising:
(i) contacting a senescent cell with the polynucleotide of any one of claims 1-18, the vector of any one of claim 35, or 37-42, the cell of any one of claims 43-45, the composition of claim 46, the rAAV of claim 47 or 48, or the pharmaceutical composition of claim 49 in an amount effective to partially reprogram the senescent cell; and
(ii) contacting the senescent cell the polynucleotide of any one of claims 19-34, or the vector of any one of claim 36, or 37-42 in an amount effective to inhibit SASP activity in the senescent cell.

85. A method for rejuvenating senescent cells in a subject to a non-senescent stage phenotype, the method comprising:
(i) administering to a subject in need thereof a therapeutically effective amount of the polynucleotide of any one of claims 1-34, the vector of any one of claim 35, or 37-42, the cell of any one of claims 43-45, the composition of claim 46, the rAAV of claim 47 or 48, or the pharmaceutical composition of claim 49, to partially reprogram the senescent cells; and
(ii) administering to the subject a therapeutically effective amount of the polynucleotide of any one of claims 19-34, or the vector of any one of claims 36-42, to inhibit SASP activity.

86. The method of claim 85, wherein the administering is by systemic administration.

87. A method for increasing age-related survival of a subject, the method comprising:
(i) administering to a subject in need thereof a therapeutically effective amount of the polynucleotide of any one of claims 1-34, the vector of any one of claims 35-42, the cell of any one of claims 43-45, the composition of claim 46, the rAAV of claim 47 or 48, or the pharmaceutical composition of claim 49, and
(ii) reprogramming senescent cells in the subject, whereby the age-related survival of the subject is increased.

88. The method of claim 87, wherein the administering is by systemic administration.

89. A method for treating cellular senescence-related aging in a subject in need thereof, comprising administering to subject a therapeutically effective amount of the polynucleotide of any one of claims 1-34, the vector of any one of claims 35-42, the cell of any one of claims 43-45, the composition of claim 46, the rAAV of claim 47 or 48, or the pharmaceutical composition of claim 49.

90. The method of claim 89, wherein the administering is by systemic administration.

91. A method of treating Hutchinson-Gilford Progeria Syndrome in a subject in need thereof comprising administering to subject a therapeutically effective amount of the polynucleotide of any one of claims 1-34, the vector of any one of claims 35-42, the cell of any one of claims 43-45, the composition of claim 46, the rAAV of claim 47 or 48, or the pharmaceutical composition of claim 49.

92. The method of claim 91, wherein the administering is by systemic administration.

93. The method of claim 91 or 92, wherein the subject is a human.

94. The method of any one of claims 91-93, wherein the administration induces increased epidermal thickness, reduced mesangial area in the kidney, increased structural arrangement of hepatocytes in the liver, rescued lymphoid depletion in germinal centers of the spleen, reduced level of inflammatory markers, increased number of hematopoietic stem cells, restoration of bone marrow tissue and/or an increased activation of DNA repair pathways in the subject.

95. The method of claim 94, wherein the reduced level of inflammatory markers comprises reduced neutrophil invasion in the liver and a reduced inflammatory signature in the spleen.

96. Use of the polynucleotide of any one of claims 1-34, the vector of any one of claims 35-42, the cell of any one of claims 43-45, the composition of claim 46, the rAAV of claim 47 or 48, or the pharmaceutical composition of claim 49 for treating cellular senescence-related aging.

97. Use of the polynucleotide of any one of claims 1-34, the vector of any one of claims 35-42, the cell of any one of claims 43-45, the composition of claim 46, the rAAV of claim 47 or 48, or the pharmaceutical composition of claim 49 in the manufacture of a medicament for treating cellular senescence-related aging.

98. An article of manufacture comprising the polynucleotide of any one of claims 1-34, the vector of any one of claims 35-42, the cell of any one of claims 43-45, the composition of claim 46, the rAAV of claim 47 or 48, or the pharmaceutical composition of claim 49.
